(19) United States
(12) Patent Application Publication
(10) Pub. No.: US 2019/0185471 A1
(43) Pub. Date: Jun. 20, 2019

(54) SOLID FORMS OF VENETOCLAX AND PROCESSES FOR THE PREPARATION OF VENETOCLAX

(71) Applicant: DR. REDDY'S LABORATORIES LIMITED, Hyderabad (IN)

(72) Inventors: Subba Reddy PEDDI REDDY, Hyderabad (IN); Mohan Kumar KOTTUR, Hyderabad (IN); Ramprasad JURUPULA, Khammam (IN); Mohammed Azeezulla BAIG, Hyderabad (IN); Ramesh CHAKKA, Hyderabad (IN); Rajesh THI PPARABOINA, Manchiryal (IN); Vishweshwar PEDDY, Hyderabad (IN); Pallavi RAO, Hyderabad (IN); Srinivas ORUGANTI, Hyderabad (IN); Vilas Hareshwar DAHANUKAR, Khajaguda (IN)

(73) Assignee: Dr. Reddy's Laboratories Limited, Hyderabad (IN)

(21) Appl. No.: 16/307,401

(22) PCT Filed: Jun. 8, 2017
(86) PCT No.: PCT/IB2017/053384

§ 371 Date: Dec. 5, 2018

(30) Foreign Application Priority Data
Jun. 9, 2016 (IN) 201641019846
Jul. 1, 2016 (IN) 201641022742
Nov. 4, 2016 (IN) 201641037731
Jan. 19, 2017 (IN) 201741002081

(51) Int. Cl. C07D 471/04

(52) U.S. Cl. CPC C07D 471/04; C07B 2200/13

(57) ABSTRACT

Aspects of the present application relate to solid forms of Venetoclax and preparative processes thereof. Specific aspects relate to an amorphous form of Venetoclax, its solid dispersion and crystalline forms of Venetoclax or salts thereof. Further aspects of the present application relate to processes for the preparation of Venetoclax.

### INTRODUCTION

Aspects of the present application relate to solid forms of Venetoclax and preparative processes thereof. Specific aspects relate to an amorphous form of Venetoclax, its solid dispersion and crystalline forms of Venetoclax or salts thereof. Further aspects of the present application relate to processes for the preparation of Venetoclax.

The drug compound having the adopted name “Venetoclax” has chemical name: 4-(4-{[2-(4-chlorophenyl)-4,4dimethylcyclohex-1-en-1-yl]methyl}piperazin-1-yl)-N-({3-nitro-4-[(tetrahydro-2H-pyran-4ylmethyl)amino]phenyl}sulfonyl)-2-(1H-pyrrolo[2,3-b]pyridin-5-yloxy)benzamide) as below.

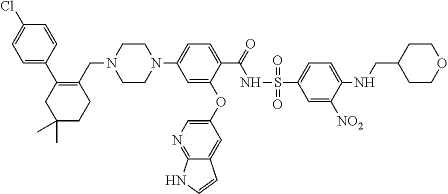 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Venetoclax is a selective and orally bioavailable small-molecule inhibitor of BCL-2, an anti-apoptotic protein. Overexpression of BCL-2 has been demonstrated in CLL cells where it mediates tumor cell survival and has been associated with resistance to chemotherapeutics. Venetoclax helps restore the process of apoptosis by binding directly to the BCL-2 protein, displacing pro-apoptotic proteins like BIM, triggering mitochondrial outer membrane permeabilization and the activation of caspases. In nonclinical studies, Venetoclax has demonstrated cytotoxic activity in tumor cells that overexpress BCL-2.

Venetoclax is approved in US as VENCLEXTA tablet for oral administration for the treatment of patients with chronic lymphocytic leukemia with 17p deletion, as detected by an FDA approved test, who have received at least one prior therapy. This indication is approved under accelerated approval based on overall response rate. VENCLEXTA is available as 10, 50 and 100 mg tablets with dosage of 20 mg once daily for 7 days, followed by a weekly ramp-up dosing schedule to the recommended daily dose of 400 mg.

U.S. Pat. No. 8,546,399 B2 discloses Venetoclax and its pharmaceutical compositions. U.S. Pat. No. 8,546,399 B2 illustrates the usefulness of Venetoclax as an inhibitor of BCL-2 protein. Further, it discloses preparative methods for the preparation of compounds disclosed therein including Venetoclax by reacting 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoicacid with 3-nitro-4-(((tetrahydro-2H-pyran-4-yl)methyl)amino)benzene sulfonamide as depicted in scheme-1. The product was isolated by chromatography from 25-100% ethyl acetate/hexane and then with 10% methanol/ethyl acetate with 1% acetic acid as a white solid.

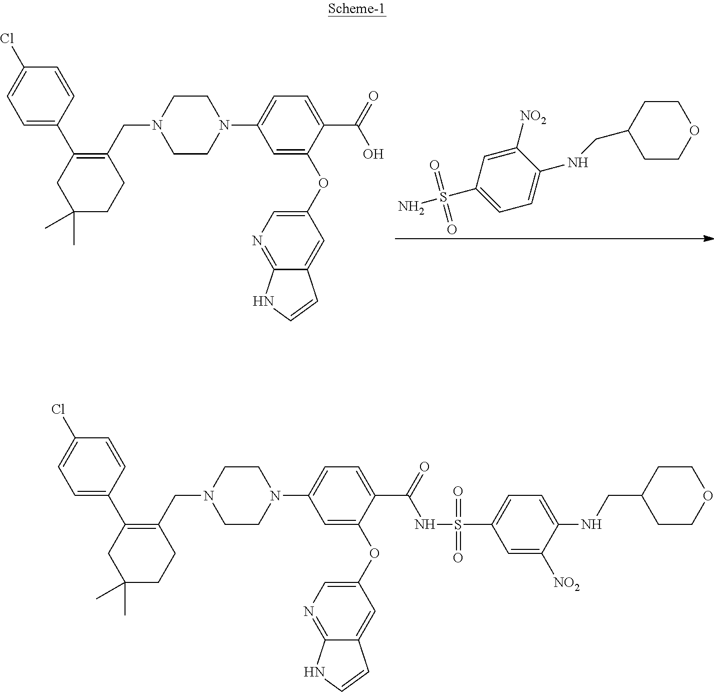 [SMILES: CC1(C)CCC(CN2CCN(c3ccc(C(=O)O)c(Oc4cnc5[nH]ccc5c4)c3)CC2)=C(c2ccc(Cl)cc2)C1 ; NS(=O)(=O)c1ccc(NCC2CCOCC2)c([N+](=O)[O-])c1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

An identical synthetic approach to scheme-1 is disclosed in the PCT applications, WO 2012121758 A1 and WO 2012058392 A1 which also describe the solid dispersions of various BCL-2 protein inhibitors with at least one water soluble polymer and at least one surfactant in essentially non-crystalline form including Venetoclax.

U.S. Pat. No. 8,722,657 B2 also describes a process similar to scheme-1 for the preparation of Venetoclax as compound L (compound 1 free base). Further, it describes various crystalline forms A to N of Venetoclax including solvated and non-solvated forms and salts of Venetoclax including hydrochloride and sulfate.

U.S. Pat. No. 8,722,657 B2 discloses that Venetoclax is obtained in amorphous state through the synthesis disclosed therein. Further, it indicates that amorphous form of Venetoclax may not be suitable as an active pharmaceutical ingredient for various types of downstream formulations. Also, it discloses that it is difficult and expensive to purify amorphous form of Venetoclax, which can present process control problems.

U.S. Pat. No. 9,006,438 B2 describes an improved process for the preparation of Venetoclax through the formation of tert. Butyl 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl) benzoate by reacting tert. butyl ester of 2-((1H-pyrrolo[2,3-b]pyridin-5-yl)oxy)-4-bromobenzoate with 1-((4′-chloro-5,5-dimethyl-3,4,5,6-tetrahydro-[1,1′-biphenyl]-2-yl)methyl)piperazine in the presence of tert. butoxide salt as depicted in scheme-2.

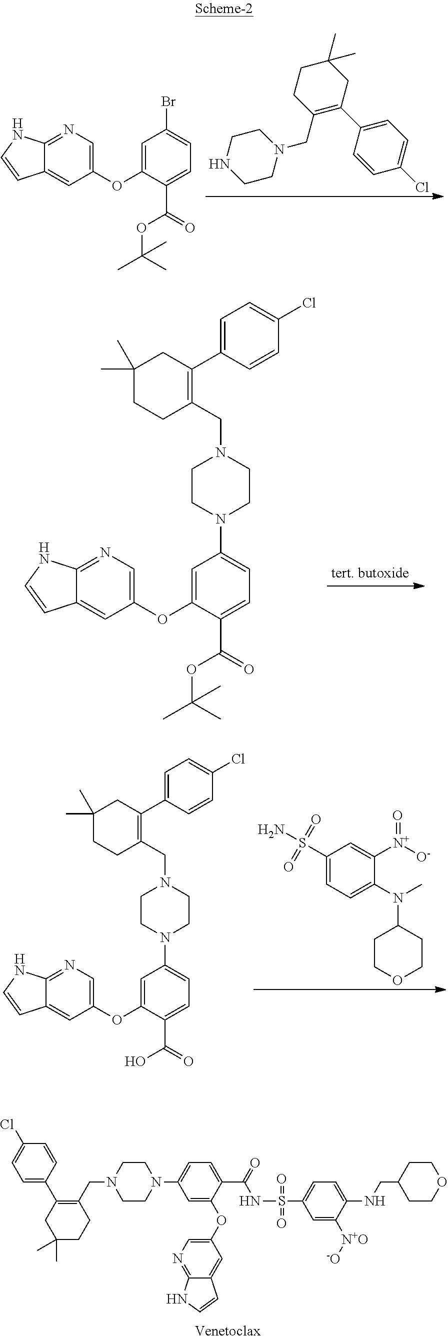

Therefore, there remains a need for alternate solid forms of Venetoclax and preparative processes thereof. Particularly, an amorphous form of a drug may exhibit a higher bioavailability than its crystalline counterparts, which leads to the selection of the amorphous form as the final drug substance for pharmaceutical dosage form development.

None of these arts disclose an amenable and/or scalable solid form of Venetoclax, which are stable enough and suitable for formulating as drug product. Hence, there remains a need for alternate solid forms which can overcome said disadvantages of the prior art and their preparative processes in a more cost effective and industrially viable manner.

None of the prior art processes disclose an amenable and commercially scalable synthetic process for the manufacture of Venetoclax. Hence, there remains a need for an alternate process for its preparation in a more cost effective and industrially viable manner.

### SUMMARY

In an aspect, the present application provides a stable amorphous form of Venetoclax.

In another aspect, the present application provides a process for the preparation of an amorphous form of Venetoclax, comprising the steps of:
a) providing a solution of Venetoclax in a suitable solvent or a mixture thereof;
b) removing the solvent from the solution obtained in step a); and
c) isolating the amorphous form of Venetoclax.
d) optionally combining amorphous form of step c) with at least one pharmaceutically acceptable excipient.

In another aspect, the present application provides a process for the preparation of amorphous form of Venetoclax, comprising the steps of:
a) providing a solution of Venetoclax in a suitable solvent or a mixture thereof.
b) contacting the solution of step a) with an anti-solvent.
c) isolating amorphous form of Venetoclax.

In another aspect, the present application provides amorphous solid dispersion of Venetoclax together with at least one pharmaceutically acceptable excipient.

In a specific aspect, the present application provides amorphous solid dispersion of Venetoclax together with one or more water soluble polymers without surfactant.

In a specific aspect, the present application provides amorphous solid dispersion of Venetoclax together with water insoluble polymer with or without surfactant.

In a specific aspect, the present application provides amorphous solid dispersion of Venetoclax together with at least one non-polymeric excipient with or without surfactant.

In a specific aspect, the present application provides amorphous solid dispersion of Venetoclax together with surfactant and without water soluble polymer.

In another aspect, the present application provides a process for the preparation of an amorphous solid dispersion of Venetoclax, comprising the steps of:
a) providing a solution of Venetoclax and at least one pharmaceutically acceptable excipient in a suitable solvent or a mixture thereof;
b) removing the solvent from the solution obtained in step a), and
c) isolating the amorphous solid dispersion of Venetoclax.
d) optionally combining amorphous solid dispersion of step c) with at least one additional pharmaceutically acceptable excipient.

In an aspect, the present application provides acid addition salts of Venetoclax, wherein the acid may be selected from the group comprising of trifluoro acetic acid (TFA), oxalic acid, maleic acid, isethionic acid, ortho-phosphoric acid, citric acid, methanesulfonic acid and acetic acid.

In another aspect, the present application provides a trifluoro acetic acid (TFA) salt of Venetoclax.

In another aspect, the present application provides an oxalic acid salt of Venetoclax.

In another aspect, the present application provides a maleic acid salt of Venetoclax.

In another aspect, the present application provides an isethionic acid salt of Venetoclax.

In another aspect, the present application provides an ortho-phosphoric salt of Venetoclax.

In another aspect, the present application provides a citric acid salt of Venetoclax.

In another aspect, the present application provides a methanesulfonic acid salt of Venetoclax.

In another aspect, the present application provides an acetic acid salt of Venetoclax.

In another aspect, the present application provides a process for the preparation of salt of Venetoclax comprising the step of contacting an acid with Venetoclax, wherein acid may be selected from the group comprising of trifluoro acetic acid (TFA), oxalic acid, maleic acid, isethionic acid, ortho-phosphoric acid, citric acid, methanesulfonic acid and acetic acid.

In another aspect, the present application provides a process for the preparation of Venetoclax, comprising the step of converting a salt of Venetoclax into its free form, wherein the salt may be selected from the group comprising of trifluoro acetic acid (TFA), oxalic acid, maleic acid, isethionic acid, ortho-phosphoric acid, citric acid, methanesulfonic acid and acetic acid.

In another aspect, the present application provides a crystalline Form RT1 of Venetoclax characterized by a PXRD pattern comprising the peaks at about 4.39 and 8.56±0.2° 2θ.

In another aspect, the present application provides a crystalline Form RT2 of Venetoclax characterized by a PXRD pattern comprising the peaks at about 6.55, 19.37, 23.07, 26.82 and 28.70±0.2° 2θ.

In another aspect, the present application provides a crystalline Form RT3 of Venetoclax characterized by a PXRD pattern comprising the peaks at about 6.30, 12.57 and 20.06±0.2° 2θ.

In an aspect, the present application provides a crystalline Form RT4 of Venetoclax characterized by a PXRD pattern comprising the peaks at about 4.55 and 5.10±0.2° 2θ.

In another aspect, the present application provides a crystalline Form RT5 of Venetoclax characterized by a PXRD pattern comprising the peaks at about 5.51 and 25.00±0.2° 2θ.

In another aspect, the present application provides a process for the preparation of crystalline Form RT1 of Venetoclax, comprising the step of contacting Venetoclax with benzyl alcohol.

In another aspect, the present application provides a process for the preparation of crystalline Form RT2 of Venetoclax, comprising the steps of:
a) dissolving or suspending Venetoclax in methyl isopropyl ketone or a mixture thereof
b) optionally, contacting the solution of step a) with an anti-solvent
c) isolating crystalline Form RT2 of Venetoclax

In another aspect, the present application provides a process for the preparation of crystalline Form RT2 of Venetoclax, comprising the steps of:
a) dissolving or suspending Venetoclax in methyl-tertiary butyl ether or a mixture thereof
b) optionally, contacting the solution of step a) with an anti-solvent
c) isolating crystalline Form RT2 of Venetoclax

In another aspect, the present application provides a process for the preparation of crystalline Form RT3 of Venetoclax, comprising the steps of:
a) providing a solution of Venetoclax in methylene chloride or a mixture thereof
b) optionally washing the solution of step a) with water
c) removing the solvent of step a) to obtain crystalline Form RT3 of Venetoclax.

In another aspect, the present application provides a process for the preparation of crystalline Form RT4 of Venetoclax, comprising the steps of:
a) dissolving or suspending Venetoclax in methyl iso-butyl Ketone or a mixture thereof
b) optionally, contacting the solution of step a) with an anti-solvent
c) isolating crystalline Form RT4 of Venetoclax

In another aspect, the present application provides a process for the preparation of crystalline Form RT5 of Venetoclax, comprising the steps of:
a) dissolving or suspending Venetoclax in 1,4-dioxane or mixture thereof.
b) optionally, contacting the solution of step a) with an anti-solvent
c) isolating crystalline Form RT5 of Venetoclax.

In an aspect, the present application provides a process for the preparation of Venetoclax, comprising the steps of
a) hydrolysis of the cyano compound of formula (II) to obtain corresponding carboxylic acid or its ester of formula (III);

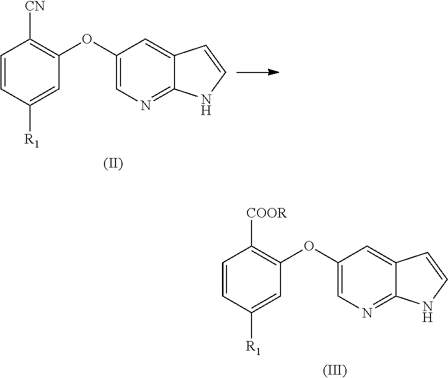

b) converting the carboxylic acid or its ester of formula (III) into Venetoclax.
   Wherein R may be selected from the group comprising of hydrogen, alkyl, aryl, arylalkyl or heteroaryl; R₁ may be selected from the group comprising of leaving group such as a halogen, optionally protected piperazine or a group of formula (A).

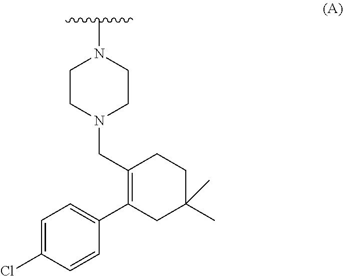 [SMILES: *N1CCN(CC2=C(c3ccc(Cl)cc3)CC(C)(C)CC2)CC1]

In an alternate aspect, the present application provides a process for the preparation of Venetoclax, comprising the steps of
a) hydrolysis of the cyano compound of formula (II) to obtain corresponding amide of formula (X);

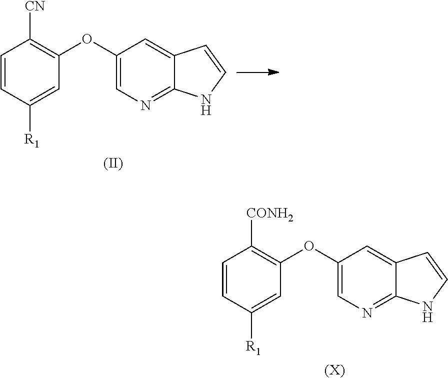

b) converting the amide of formula (X) into Venetoclax.
   Wherein R₁ may be selected from the group comprising of leaving group such as a halogen, optionally protected piperazine or a group of formula (A).

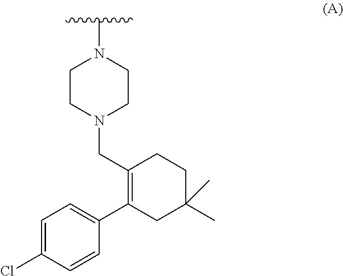 [SMILES: *N1CCN(CC2=C(c3ccc(Cl)cc3)CC(C)(C)CC2)CC1]

In another aspect, the present application provides intermediate compounds of formula (IIa), formula (IIb) formula (IIc) useful to produce Venetoclax and intermediate compounds of formula (VIIb) and (VIIc) useful to produce compounds of formula (IIb) and (IIc), wherein X is any halogen such as fluorine, chlorine, Bromine or Iodine and P is hydrogen or any nitrogen protecting group such as BoC or Cbz.

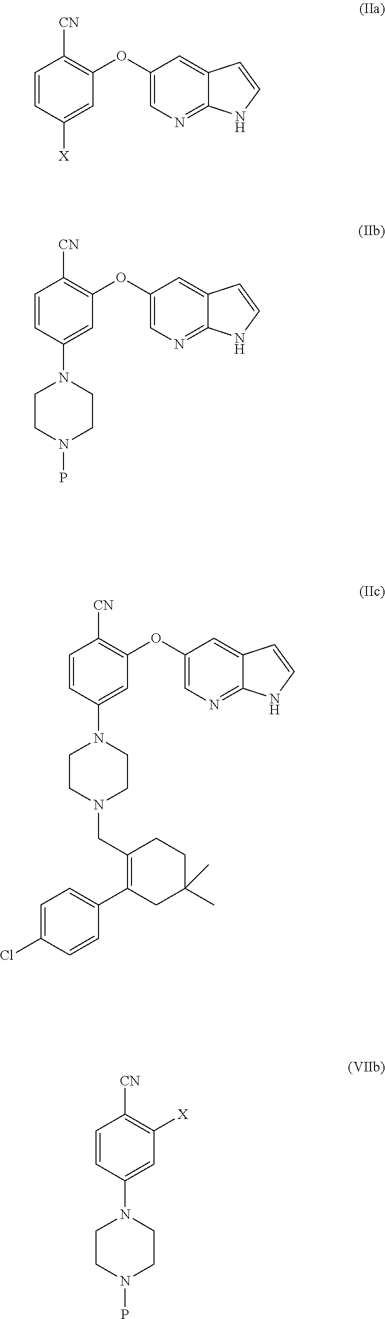

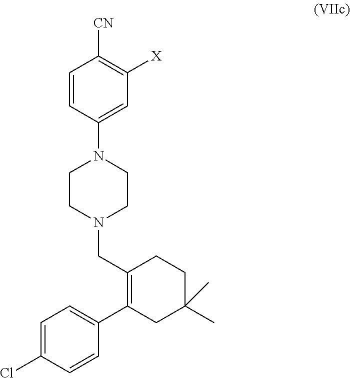

In another aspect, the present application provides intermediate compounds of formula (Xa), (Xb), (Xc) useful to produce Venetoclax and intermediate compounds of formula (XIb) and formula (XIc) useful to produce intermediate compounds of formula (Xb) and (Xc); wherein X is any halogen such as fluorine, chlorine, Bromine or Iodine and P is hydrogen or any nitrogen protecting group such as BoC or Cbz.

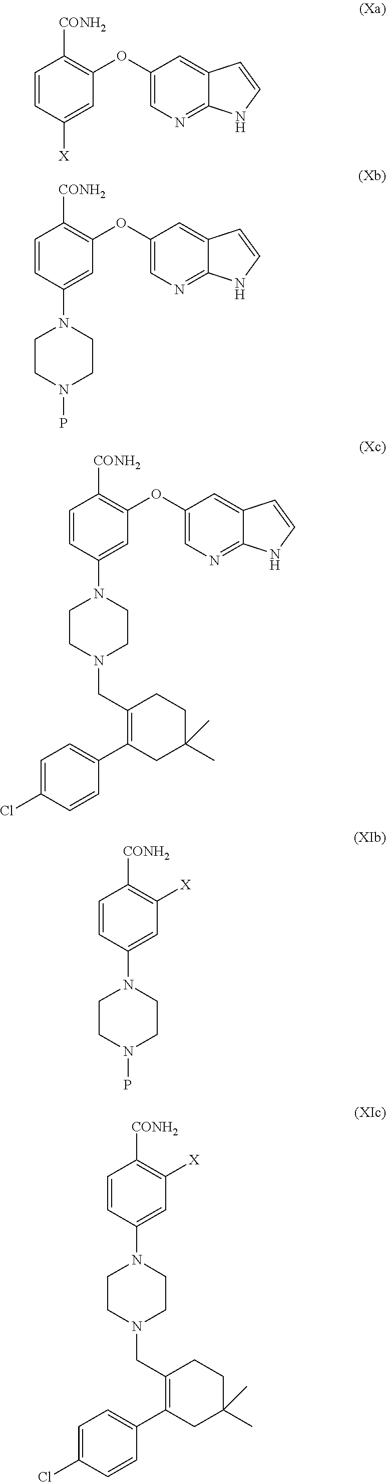

In another aspect, the present application provides a process for the preparation of Venetoclax, comprising the step of converting the compound of formula (IIIb) to Venetoclax;

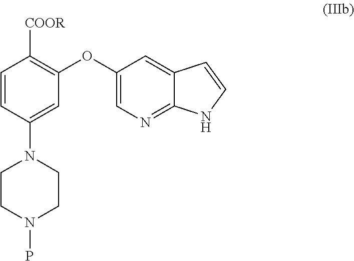

wherein P is hydrogen or any nitrogen protecting group such as BoC or Cbz and R may be selected from the group comprising of hydrogen, alkyl, aryl, arylalkyl or heteroaryl.

In another aspect, the present application provides a process for the preparation of Venetoclax, comprising the step of
a) reacting compound of formula (IIIa) with optionally protected piperazine (or) reacting compound of formula (IV) with optionally protected 1H-pyrrolo[2,3-b]pyridin-5-ol or its reactive derivative to obtain compound of formula (IIIb).

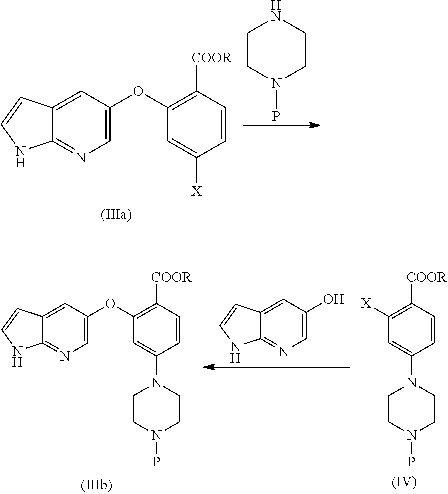

b) converting the compound of formula (IIIb) to Venetoclax.

In another aspect, the present application provides a process for the preparation of Venetoclax, comprising the step of converting the compound of formula (III) to compound of formula (V),

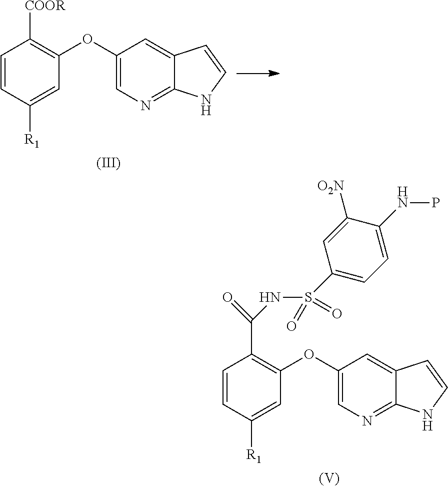

wherein R may be selected from the group comprising of hydrogen, alkyl, aryl, arylalkyl or heteroaryl; R₁ may be selected from the group comprising of leaving group such as a halogen or optionally protected piperazine and P is hydrogen, any nitrogen protecting group such as BoC or Cbz or a group of formula (B)

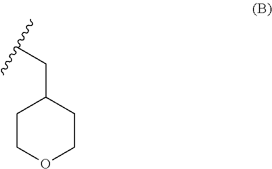 [SMILES: *CC1CCOCC1]

In another aspect, the present application provides a process for the preparation of Benzoate compound of formula (III) comprising the step of reacting 2-halo benzoate of formula (VI) with optionally protected 1H-pyrrolo[2,3-b]pyridin-5-ol or its reactive derivative

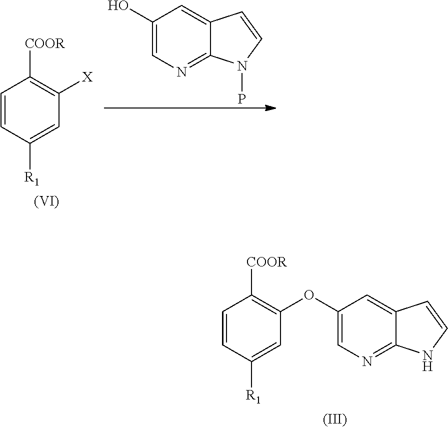

Wherein R may be selected from the group comprising of alkyl, aryl, arylalkyl or heteroaryl; R₁ may be selected from the group comprising of leaving group such as a halogen, optionally protected piperazine or a group of formula (A).

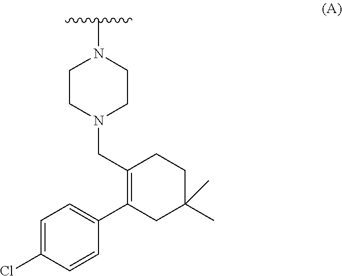 [SMILES: *N1CCN(CC2=C(c3ccc(Cl)cc3)CC(C)(C)CC2)CC1]

In another aspect, the present application provides a process for the preparation of cyano compound of formula (II) comprising the step of reacting 2-halo benzonitrile of formula (VII) with optionally protected 1H-pyrrolo[2,3-b]pyridin-5-ol or its reactive derivative

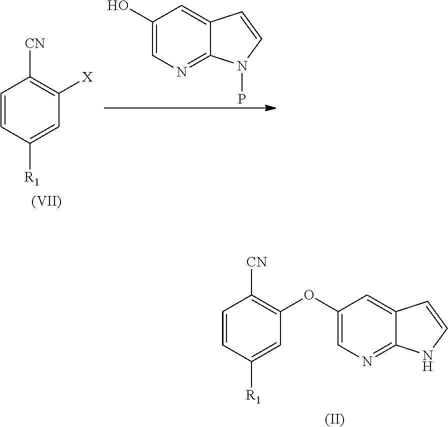

Wherein R₁ may be selected from the group comprising of leaving group such as a halogen, optionally protected piperazine or a group of formula (A).

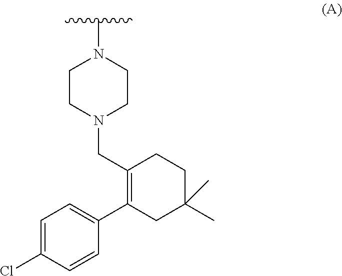 [SMILES: *N1CCN(CC2=C(c3ccc(Cl)cc3)CC(C)(C)CC2)CC1]

In another aspect, the present application provides a process for the preparation of compound of formula (IIa) or (IIIa), comprising the step of reacting a 2,4-dihalo benzene derivative of formula (VIIa) or (VIa), respectively with an optionally protected 1H-pyrrolo[2,3-b]pyridin-5-ol or its reactive derivative in the presence of suitable solvent system comprising 1,4-dioxane; wherein R₃ may be CN or COOR; R may be selected from the group comprising of alkyl, aryl, arylalkyl or heteroaryl and X is any halogen selected from the group comprising of fluorine, chlorine, Bromine or Iodine.

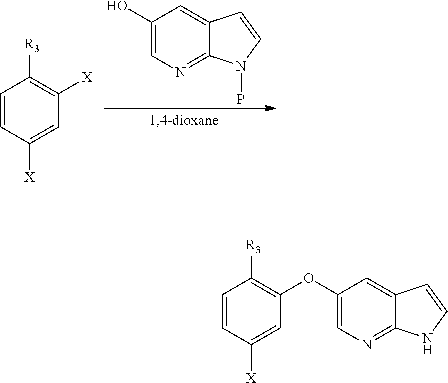

### BRIEF DESCRIPTION OF THE DRAWING

FIG. 1 is an illustrative X-ray powder diffraction pattern of amorphous form of Venetoclax prepared by the method of Example No 1.

FIG. 2 is an illustrative X-ray powder diffraction pattern of amorphous form of Venetoclax prepared by the method of Example No 2.

FIG. 3 is an illustrative X-ray powder diffraction pattern of amorphous form of Venetoclax prepared by the method of Example No 3.

FIG. 4 is an illustrative X-ray powder diffraction pattern of amorphous solid dispersion of Venetoclax with Syloid prepared by the method of Example No 4.

FIG. 5 is an illustrative X-ray powder diffraction pattern of amorphous solid dispersion of Venetoclax with povidone K-30 prepared by the method of Example No 5.

FIG. 6 is an illustrative X-ray powder diffraction pattern of amorphous solid dispersion of Venetoclax with povidone K-30 and Syloid prepared by the method of Example No 6.

FIG. 7 is an illustrative X-ray powder diffraction pattern of amorphous solid dispersion of Venetoclax with HPMC-AS prepared by the method of Example No 7.

FIG. 8 is an illustrative X-ray powder diffraction pattern of amorphous solid dispersion of Venetoclax with Eudragit-RLPO prepared by the method of Example No 8.

FIG. 9 is an illustrative X-ray powder diffraction pattern of amorphous solid dispersion of Venetoclax with HPC-L prepared by the method of Example No 9.

FIG. 10 is an illustrative X-ray powder diffraction pattern of amorphous form of Venetoclax prepared by the method of Example No 10.

FIG. 11 is an illustrative X-ray powder diffraction pattern of amorphous solid dispersion of Venetoclax with Soluplus® prepared by the method of Example No 11.

FIG. 12 is an illustrative X-ray powder diffraction pattern of crystalline Trifluoroacetic acid (TFA) salt of Venetoclax prepared by the method of Example No 12.

FIG. 13 is an illustrative X-ray powder diffraction pattern of crystalline Oxalic acid salt of Venetoclax prepared by the method of Example No 13.

FIG. 14 is an illustrative X-ray powder diffraction pattern of crystalline Maleic acid salt of Venetoclax prepared by the method of Example No 14.

FIG. 15 is an illustrative X-ray powder diffraction pattern of crystalline Isethionic acid salt of Venetoclax prepared by the method of Example No 15.

FIG. 16 is an illustrative X-ray powder diffraction pattern of crystalline hydrochloride salt of Venetoclax prepared by the method of Example No 16.

FIG. 17 is an illustrative X-ray powder diffraction pattern of crystalline Ortho-phosphoric acid salt of Venetoclax prepared by the method of Example No 17.

FIG. 18 is an illustrative X-ray powder diffraction pattern of crystalline Citric acid salt of Venetoclax prepared by the method of Example No 18.

FIG. 19 is an illustrative X-ray powder diffraction pattern of crystalline Methanesulfonic acid salt of Venetoclax prepared by the method of Example No 19.

FIG. 20 is an illustrative X-ray powder diffraction pattern of crystalline acetic acid salt of Venetoclax prepared by the method of Example No 20.

FIG. 21 is an illustrative X-ray powder diffraction pattern of crystalline Form RT1 of Venetoclax prepared by the method of Example No 21.

FIG. 22 is an illustrative X-ray powder diffraction pattern of crystalline Form RT2 of Venetoclax prepared by the method of Example No 22.

FIG. 23 is an illustrative X-ray powder diffraction pattern of crystalline Form RT3 of Venetoclax prepared by the method of Example No 23.

FIG. 24 is an illustrative X-ray powder diffraction pattern of amorphous form of Venetoclax prepared by the method of Example No 24.

FIG. 25 is an illustrative X-ray powder diffraction pattern of crystalline Form RT4 of Venetoclax prepared by the method of Example No 25.

FIG. 26 is an illustrative X-ray powder diffraction pattern of crystalline Form RT5 of Venetoclax prepared by the method of Example No 26.

FIG. 27 is an illustrative X-ray powder diffraction pattern of crystalline Form RT2 of Venetoclax prepared by the method of Example No 27.

FIG. 28 is an illustrative X-ray powder diffraction pattern of crystalline Venetoclax prepared by the method of Example No 44.

### DETAILED DESCRIPTION

In an aspect, the present application provides a stable amorphous form of Venetoclax.

The present application provides a stable amorphous form of Venetoclax devoid of the problems indicated in the prior art and suitable for powder handling and downstream processes. Amorphous form of Venetoclax of the present application which was surprisingly found to be highly stable under mechanical stress such as grinding and milling and stable under hygroscopic conditions such as higher relative humidity conditions of more than 60% RH.

In an embodiment, the present application provides a stable amorphous form of Venetoclax with less than 5% of crystallinity, preferably with less than 1% crystallinity and more preferably with less than 0.5% crystallinity as per X-ray diffraction analysis.

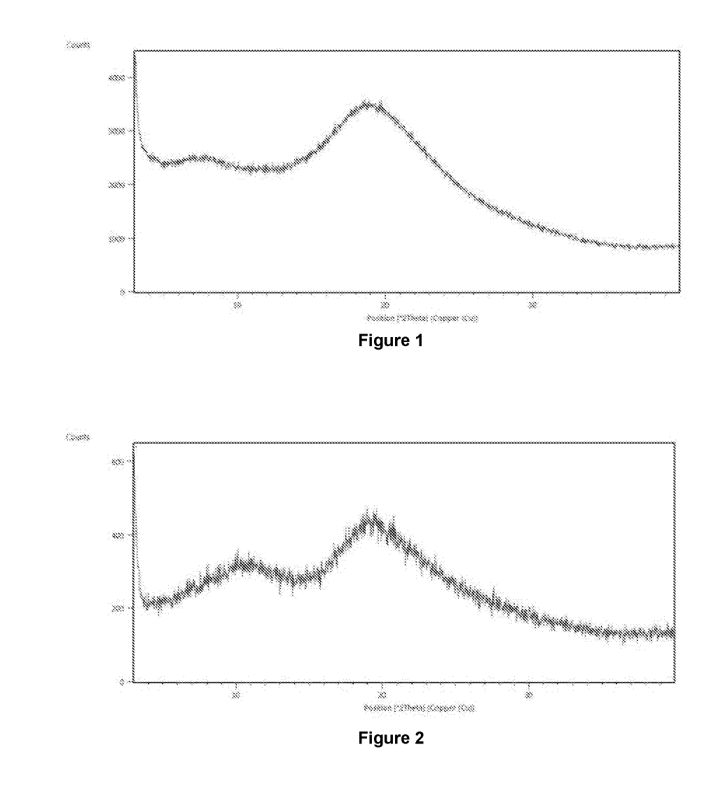

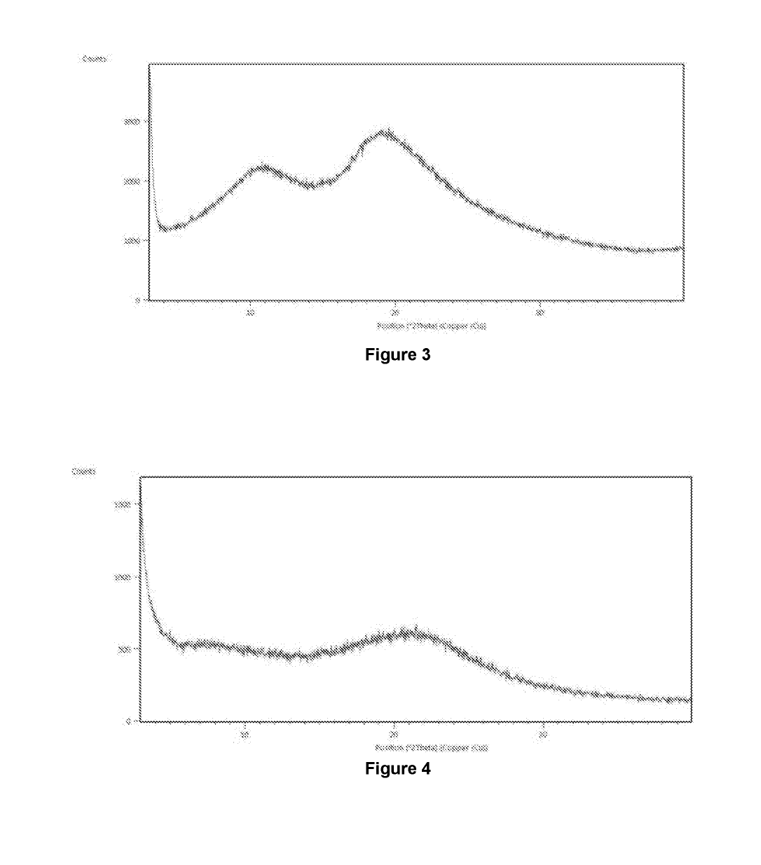

In an embodiment, the present application provides an amorphous form of Venetoclax characterized by a powder X-ray diffraction (PXRD) pattern, substantially as illustrated by FIG. 1, 2, 3, 10 or 24.

In another aspect, the present application provides a process for the preparation of an amorphous form of Venetoclax, comprising the steps of:
a) providing a solution of Venetoclax in a suitable solvent or a mixture thereof;
b) removing the solvent from the solution obtained in step a); and
c) isolating the amorphous form of Venetoclax.
d) optionally combining amorphous form of step c) with at least one pharmaceutically acceptable excipient.

In an embodiment, suitable solvent at step a) of this aspect may be selected from C₁-C₆ alcohols, C₃-C₆ ketones, C₅-C₈ aliphatic or aromatic hydrocarbons, C₃-C₆ esters, C₂-C₆ aliphatic or cyclic ethers, C₂-C₆ nitriles, halogenated hydrocarbons, water or mixtures thereof.

In preferred embodiment, the suitable solvent may be selected from the group comprising of alcohol solvents such as methanol, ethanol, 2-propanol, 1-butanol, 2-butanol, 1-pentanol, 2-pentanol, 3-pentanol; dichloromethane; tetrahydrofuran; ketone solvents such as acetone, methyl ethyl ketone, methyl isobutyl ketone; esters solvents such as methyl acetate, ethyl acetate, isopropyl acetate; water and mixtures thereof.

In an embodiment, providing a solution at step a) may be carried out by dissolving Venetoclax in a suitable solvent or by taking the reaction mixture containing Venetoclax directly. In an embodiment, a solution of Venetoclax can be prepared at any suitable temperatures, such as about 0° C. to about the reflux temperature of the solvent used. Stirring and heating may be used to reduce the time required for the dissolution process.

In an embodiment, a solution of Venetoclax may be filtered to make it clear, free of unwanted particles. In embodiments, the obtained solution may be optionally treated with an adsorbent material, such as carbon and/or hydrose, to remove colored components, etc., before filtration.

In an embodiment, removal of solvent at step b) may be carried out by methods known in the art or any procedure disclosed in the present application. In preferred embodiments, removal of solvent may include, but not limited to: solvent evaporation under atmospheric pressure or reduced pressure/vacuum such as a rotational distillation using buchi rotavapor, spray drying, freeze drying, thin film drying, agitated thin film drying, rotary vacuum paddle dryer (RVPD) and the like.

In preferred embodiment, the solvent may be removed under reduced pressures and at temperatures of less than about 100° C., less than about 60° C., less than about 40° C., less than about 20° C., less than about 0° C., less than about −20° C., less than about −40° C., less than about −60° C., less than about −80° C., or any other suitable temperatures.

In an embodiment, the isolation of an amorphous form of Venetoclax at step c) involves recovering the solid obtained in step b). The solid obtained from step b) may be recovered using techniques such as by scraping, or by shaking the container, or adding solvent to make slurry followed by filtration, or other techniques specific to the equipment used. In an embodiment, the amorphous form of Venetoclax obtained from step b) may be optionally dried before or after isolating it at step c).

Amorphous form of Venetoclax obtained at step c) may be optionally combined with at least one pharmaceutically acceptable excipient at step d).

In an embodiment, amorphous form of Venetoclax may be combined with excipient using a technique known in art or by the procedures disclosed in the present application.

In preferred embodiment, amorphous form of Venetoclax may be combined with excipient either by physical blending of both the solid components or by suspending both the components in a suitable solvent and conditions, such that both the components remain unaffected. Blending may be carried out using techniques known in art such as rotatory cone dryer, fluidized bed dryer or the like optionally under reduced pressure/vacuum or inert atmosphere such nitrogen at suitable temperature and sufficient time to obtain uniform composition of amorphous form of Venetoclax and at least one pharmaceutically acceptable excipient.

In an embodiment, amorphous form of Venetoclax may be combined with the excipient by evaporating the suspension or solution of amorphous form of Venetoclax and at least one pharmaceutically acceptable excipient.

In an embodiment, pharmaceutically acceptable excipient may include, but not limited to an inorganic oxide such as SiO₂, TiO₂, ZnO₂, ZnO, Al₂O₃ and zeolite; a water insoluble polymer is selected from the group consisting of cross-linked polyvinyl pyrrolidinone, cross-linked cellulose acetate phthalate, cross-linked hydroxypropyl methyl cellulose acetate succinate, microcrystalline cellulose, polyethylene/polyvinyl alcohol copolymer, polyethylene/polyvinyl pyrrolidinone copolymer, cross-linked carboxymethyl cellulose, sodium starch glycolat, and cross-linked styrene divinyl benzene or any other excipient at any aspect of present application.

In preferred embodiment, pharmaceutically acceptable excipient may be selected from the group consisting of silicon dioxide, e.g. colloidal or fumed silicon dioxide or porous silica or Syloid; copolymers, such as polyethylene/polyvinyl alcohol copolymer, polyethylene/polyvinyl pyrrolidinone copolymer; and cellulose, preferably microcrystalline cellulose.

Amorphous form of Venetoclax isolated at step c) or d) may be dried in suitable drying equipment such as vacuum oven, rotatory cone dryer, air oven, fluidized bed dryer, spin flash dryer, flash dryer, or the like. The drying may be carried out at atmospheric pressure or under reduced pressures at temperatures of less than about 100° C., less than about 60° C., less than about 40° C., or any other suitable temperatures. The drying may be carried out for any time period required for obtaining a desired quality, such as from about 15 minutes to 10 hours or longer.

In another aspect, the present application provides a process for the preparation of amorphous form of Venetoclax, comprising the steps of:
a) providing a solution of Venetoclax in a suitable solvent or a mixture thereof.
b) contacting the solution of step a) with an anti-solvent.
c) isolating amorphous form of Venetoclax.

In an embodiment, step a) may be carried out by dissolving Venetoclax in a suitable solvent or a mixture thereof. Alternatively, the solution may be provided by taking the reaction mixture containing Venetoclax in solvent or a mixture of solvents.

Suitable solvent may include, but not limited to: dimethyl sulfoxide, dimethyl formamide, tetrahydrofuran or the like In an embodiment, the Venetoclax may be dissolved in the solvent, optionally under heating to obtain a homogenous solution. The solution may be filtered to make it particle free.

In an embodiment, the solution of Venetoclax of step a) may be optionally cooled to a suitable temperature before or after contacting it with anti-solvent.

In an embodiment, the solution of step a) may be cooled to temperature above the freezing point of the solvent used before contacting it with anti-solvent.

In an embodiment, the anti-solvent may be contacted at suitable temperature and concentration for the nucleation of amorphous form.

The anti-solvent may be contacted in sufficient volume to complete the formation of solids with ratio of solvent to anti-solvent of about 1:1 to 1:20.

In an embodiment, anti-solvent may be contacted in any of the modes such as addition of anti-solvent to the solution of step a) or addition of solution of step a) to the anti-solvent.

In an embodiment, the anti-solvent may be contacted for sufficient time, till the amorphous form is stable. In an embodiment, anti-solvent may be contacted with solution of step a) either by gradual addition or in single short addition such as dumping of one into the other.

Anti-solvent may include, but not limited to water; hydrocarbons such as n-hexane, n-heptane, cyclohexane or the like; ethers such as diethyl ether, di isopropyl ether, methyl tert-butyl ether; or the like.

Isolation of amorphous form of Venetoclax may be carried out by any methods known in the art or procedures described in the present application. In an embodiment, amorphous form of Venetoclax may be isolated by employing any of the techniques, but not limited to: scratching the walls of the container with a spatula, adding solvent to make slurry followed by filtration, decantation, filtration by gravity or suction, centrifugation, or other techniques specific to the equipment used and the like, and optionally washing with an anti-solvent.

In an embodiment, drying amorphous form of Venetoclax may be carried out at temperatures and times sufficient to achieve desired quality of product. Drying may be carried out for any time period required for obtaining a desired quality, such as from about 5 minutes to 10 hours or longer.

In an aspect, the present application provides amorphous solid dispersion of Venetoclax together with at least one pharmaceutically acceptable excipient.

In a specific aspect, the present application provides amorphous solid dispersion of Venetoclax together with one or more water soluble polymers without surfactant. In embodiments, water soluble polymers include, but are not limited to polyvinyl pyrrolidone, povidone K-30, povidone K-60, povidone K-90, polyvinylpyrrolidone vinylacetate, co-povidone NF, polysorbate 80, polyoxyethylene-polyoxypropylene copolymers (Poloxamer 188 or pluronic F-68), polyoxyethylene (40) stearate, polyethyene glycol monomethyl ether, polyethyene glycol, hydroxypropylmethyl cellulose phthalate, hydroxypropylmethyl cellulose, hydroxypropyl cellulose SSL (HPC-SSL), hydroxypropyl cellulose SL (HPC-SL), hydroxypropyl cellulose L (HPC-L), hydroxyethyl cellulose, Soluplus® (polyvinyl caprolactam-polyvinyl acetate-polyethylene glycol graft copolymer (PCL-PVAc-PEG)), gelucire 44/14, cyclodextrins, gelatins, D-alpha-tocopheryl polyethylene glycol 1000 succinate, Polyvinylacetal diethylamino acetate (AEA), methylcellulose, carboxymethylethylcellulose or mixture thereof.

In a specific aspect, the present application provides amorphous solid dispersion of Venetoclax together with water insoluble polymer with or without surfactant. In embodiments, water insoluble polymer include, but are not limited to polyvinyl acetate phthalate, methacrylic acid copolymer (Eudragit or Eudragit-RLPO), hydroxypropylmethyl cellulose acetate succinate (HPMC-AS), ethyl cellulose, cellulose acetate phthalate, hypromellose phthalates, syloid or mixture thereof. Surfactant include, but are not limited to polyoxyethylene glycerides, fatty acid monoesters of sorbitan, polysorbates, α-tocopheryl polyethylene glycol succinate (TPGS) or mixtures thereof.

In a specific aspect, the present application provides amorphous solid dispersion of Venetoclax together with at least one non-polymeric excipient with or without surfactant. In embodiments, non-polymeric excipient includes, but not limited to arginine, tyrosine, phenylalanine, aspartic acid, lysine, serine, threonine, glutamine, glycine, leucine, valine, alanine, proline, citric acid, stearic acid, oxalic acid, succinic acid, tartaric acid, malic acid, dextrose, sucrose, galactose, sorbitol, maltose, xylitol, mannitol, Inulin, lactose, mesoporous silica, polysorbates, urea, gelucire, cetyl alcohol, poloxamer, cremophor, cetyl stearyl alcohol or mixtures thereof. Surfactant include, but are not limited to polyoxyethylene glycerides, fatty acid monoesters of sorbitan, polysorbates, α-tocopheryl polyethylene glycol succinate (TPGS) or mixtures thereof.

In a specific aspect, the present application provides amorphous solid dispersion of Venetoclax together with surfactant and without water soluble polymer. In embodiments, surfactant include, but are not limited to polyoxyethylene glycerides, fatty acid monoesters of sorbitan, polysorbates, α-tocopheryl polyethylene glycol succinate (TPGS) or mixtures thereof.

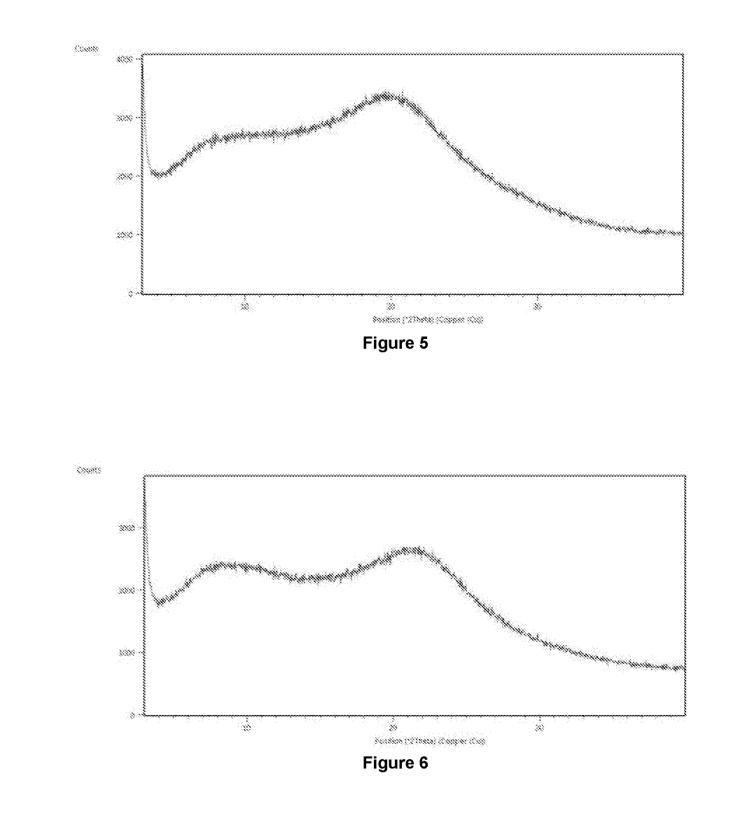

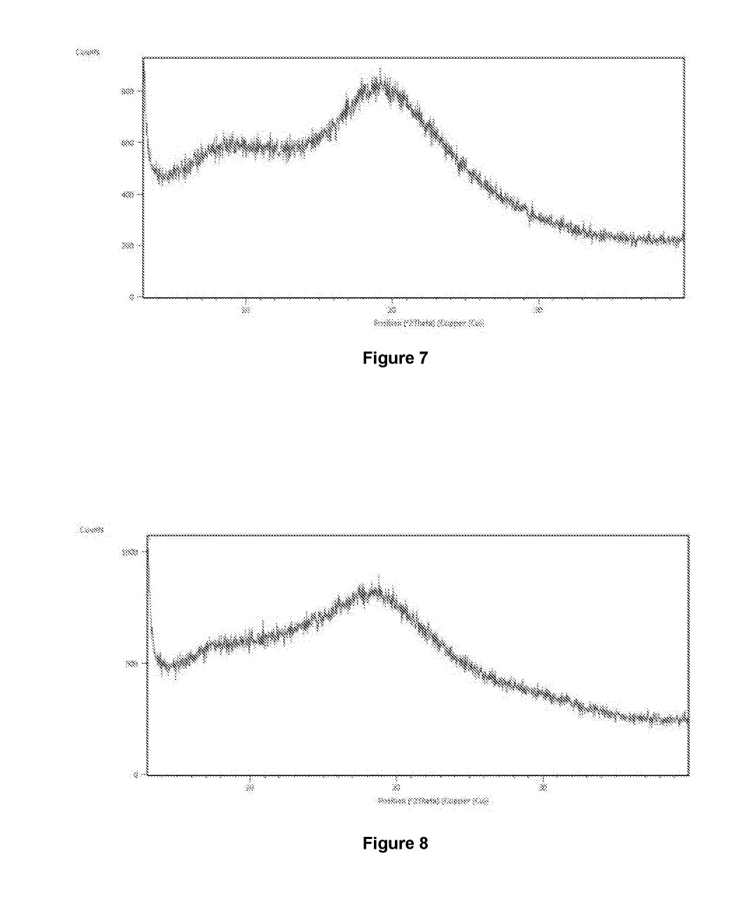

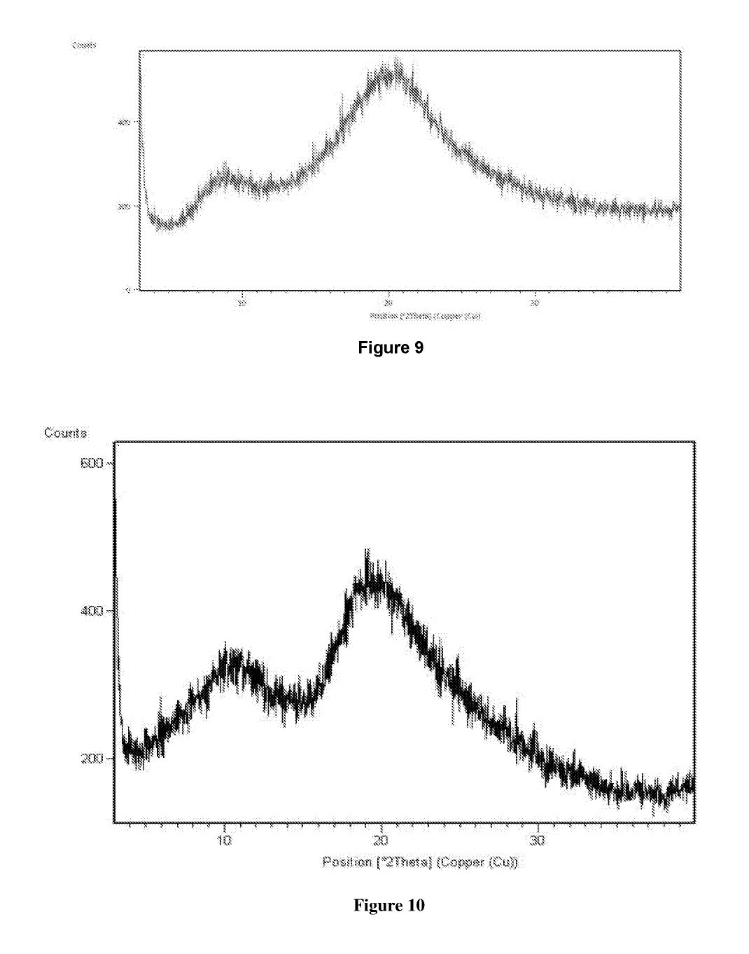

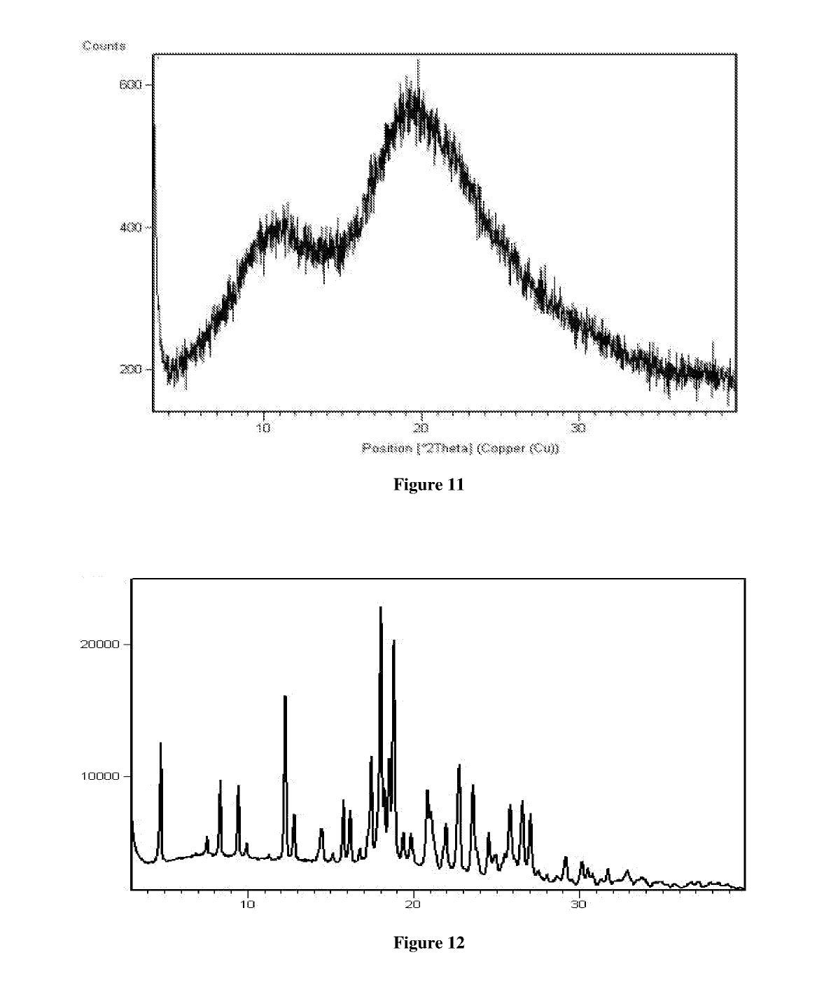

In an embodiment, the present application provides amorphous solid dispersion of Venetoclax together with at least one pharmaceutically acceptable excipient characterized by a powder X-ray diffraction (PXRD) pattern, substantially as illustrated by FIGS. 4, 5, 6, 7, 8, 9 and 11.

In another aspect, the present application provides a process for the preparation of an amorphous solid dispersion of Venetoclax, comprising the steps of:
a) providing a solution of Venetoclax and at least one pharmaceutically acceptable excipient in a suitable solvent or a mixture thereof;
b) removing the solvent from the solution obtained in step a), and
c) isolating the amorphous solid dispersion of Venetoclax.
d) optionally combining amorphous solid dispersion of step c) with at least one additional pharmaceutically acceptable excipient.

In an embodiment, suitable solvent at step a) of this aspect may be selected from C1-C6 alcohols, C3-C6 ketones, C5-C8 aliphatic or aromatic hydrocarbons, C3-C6 esters, C2-C6 aliphatic or cyclic ethers, C2-C6 nitriles, halogenated hydrocarbons, water or mixtures thereof.

In preferred embodiment, the suitable solvent may be selected from the group consisting of alcohol solvents such as methanol, ethanol, 2-propanol, 1-butanol, 2-butanol, 1-pentanol, 2-pentanol, 3-pentanol; dichloromethane, tetrahydrofuran; ketone solvents such as acetone, methyl ethyl ketone, methyl isobutyl ketone; esters solvents such as methyl acetate, ethyl acetate, isopropyl acetate; water and mixtures thereof.

In an embodiment, at least one pharmaceutically acceptable excipient of this aspect may be selected from the group consisting of polyvinyl pyrrolidone, povidone K-30, povidone K-60, Povidone K-90, polyvinylpyrrolidone vinylacetate, co-povidone NF, polyvinylacetal diethylaminoacetate (AEA®), polyvinyl acetate phthalate, polysorbate 80, polyoxyethylene-polyoxypropylene copolymers (Poloxamer® 188), polyoxyethylene (40) stearate, polyethyene glycol monomethyl ether, polyethyene glycol, poloxamer 188, pluronic F-68, methylcellulose, methacrylic acid copolymer (Eudragit or Eudragit-RLPO), hydroxypropylmethyl cellulose phthalate, hydroxypropylmethyl cellulose acetate succinate (HPMC-AS), hydroxypropylmethyl cellulose, hydroxypropyl cellulose SSL (HPC-SSL), hydroxypropyl cellulose SL (HPC-SL), hydroxypropyl cellulose L (HPC-L), hydroxyethyl cellulose, Soluplus® (polyvinyl caprolactam-polyvinyl acetate-polyethylene glycol graft copolymer (PCL-PVAc-PEG)), gelucire 44/14, ethyl cellulose, D-alpha-tocopheryl polyethylene glycol 1000 succinate, cellulose acetate phthalate, carboxymethylethylcellulose and the like; cyclodextrins, gelatins, hypromellose phthalates, sugars, polyhydric alcohols, and the like; homopolymers and copolymers of N-vinyl lactams, cellulose esters, cellulose ethers, high molecular weight polyalkylene oxides, polyacrylates, polymethacrylates, polyacrylamides, vinyl acetate polymers, graft copolymers of polyethylene glycol, polyvinyl caprolactam and polyvinyl acetate, oligo- and polysaccharides, water soluble sugar excipients, preferably having low hygroscopicity, which include, but are not limited to, mannitol, lactose, fructose, sorbitol, xylitol, maltodextrin, dextrates, dextrins, lactitol and the like; polyethylene oxides, polyoxyethylene derivatives, polyvinyl alcohols, propylene glycol derivatives and the like; organic amines such as alkyl amines (primary, secondary, and tertiary), aromatic amines, alicyclic amines, cyclic amines, aralkyl amines, hydroxylamine or its derivatives, hydrazine or its derivatives, and guanidine or its derivatives, or any other excipient at any aspect of present application. The use of mixtures of more than one of the pharmaceutical excipients to provide desired release profiles or for the enhancement of stability is within the scope of this invention. Also, all viscosity grades, molecular weights, commercially available products, their copolymers, and mixtures are all within the scope of this invention without limitation. Solid dispersions of the present application also include the solid dispersions obtained by combining Venetoclax with a suitable non-polymeric excipient by employing techniques known in the art or procedures described or exemplified in any aspect of the instant application.

In an embodiment, providing a solution at step a) may be carried out by dissolving Venetoclax and at least one pharmaceutically acceptable excipient in a suitable solvent simultaneously or by dissolving components in a suitable solvent separately to form individual solutions and combining those solutions later.

In an embodiment, a solution of Venetoclax and the excipient may be prepared at any suitable temperatures, such as about 0° C. to about the reflux temperature of the solvent used. Stirring and heating may be used to reduce the time required for the dissolution process.

In an embodiment, a solution of Venetoclax and the excipient may be filtered to make it clear, free of unwanted particles. In embodiments, the obtained solution may be optionally treated with an adsorbent material, such as carbon and/or hydrose, to remove colored components, etc., before filtration.

In an embodiment, removal of solvent at step b) may be carried out by methods known in the art or any procedure disclosed in the present application. In preferred embodiments, removal of solvent may include, but not limited to: solvent evaporation under atmospheric pressure or reduced pressure/vacuum such as a rotational distillation using buchi rotavapor, spray drying, freeze drying, agitated thin film drying and the like.

In preferred embodiment, the solvent may be removed under reduced pressures, at temperatures of less than about 100° C., less than about 60° C., less than about 40° C., less than about 20° C., less than about 0° C., less than about −20° C., less than about −40° C., less than about −60° C., less than about −80° C., or any other suitable temperatures.

In an embodiment, the isolation of an amorphous solid dispersion of Venetoclax and excipient at step c) involves recovering the solid obtained in step b). The solid obtained from step b) may be recovered using techniques such as by scraping, or by shaking the container, or adding solvent to make slurry followed by filtration, or other techniques specific to the equipment used.

In an embodiment, the amorphous solid dispersion of Venetoclax and excipient obtained from step b) may be optionally dried before or after isolating at step c).

Amorphous solid dispersion of Venetoclax obtained at step c) may be optionally combined with at least one additional pharmaceutically acceptable excipient at step d).

In an embodiment, amorphous solid dispersion of Venetoclax may be combined with additional excipient using a technique known in art or by the procedures disclosed in the present application.

In preferred embodiment, amorphous solid dispersion of the present application may be combined with additional excipient either by physical blending of both the solid components or by suspending both the components in a suitable solvent and conditions, such that both the components remain unaffected. Blending may be carried out using techniques known in art such as rotatory cone dryer, fluidized bed dryer or the like optionally under reduced pressure/vacuum or inert atmosphere such nitrogen at suitable temperature and sufficient time to obtain uniform composition of amorphous solid dispersion of Venetoclax with pharmaceutically acceptable excipient and at least one additional pharmaceutically acceptable excipient.

In an embodiment, amorphous solid dispersion of the present application may be combined with additional excipient by evaporating the suspension or solution of amorphous solid dispersion of Venetoclax and additional excipient.

In an embodiment, pharmaceutically acceptable additional excipient may be same or different from the excipient used in the preparation of amorphous solid dispersion of Venetoclax. Additional excipient may include, but not limited to an inorganic oxide such as SiO₂, TiO₂, ZnO₂, ZnO, Al₂O₃ and zeolite; a water insoluble polymer is selected from the group consisting of cross-linked polyvinyl pyrrolidinone, cross-linked cellulose acetate phthalate, cross-linked hydroxypropyl methyl cellulose acetate succinate, microcrystalline cellulose, polyethylene/polyvinyl alcohol copolymer, polyethylene/polyvinyl pyrrolidinone copolymer, cross-linked carboxymethyl cellulose, sodium starch glycolat, and cross-linked styrene divinyl benzene or any other excipient at any aspect of present application.

In preferred embodiment, pharmaceutically acceptable additional excipient may be selected from the group consisting of silicon dioxide, e.g. colloidal or fumed silicon dioxide or porous silica or syloid; copolymers, such as polyethylene/polyvinyl alcohol copolymer, polyethylene/polyvinyl pyrrolidinone copolymer; and cellulose, preferably microcrystalline cellulose.

Amorphous solid dispersion of Venetoclax isolated at step c) or d) may be dried in a suitable drying equipment such as tray dryer, vacuum oven, rotatory cone dryer, air oven, fluidized bed dryer, spin flash dryer, flash dryer, or the like. The drying may be carried out at atmospheric pressure or under reduced pressures at temperatures of less than about 100° C., less than about 60° C., less than about 40° C., or any other suitable temperatures. The drying may be carried out for any time period required for obtaining a desired quality, such as from about 15 minutes to 10 hours or longer.

In an aspect, the present application provides pharmaceutical composition comprising amorphous solid dispersion of Venetoclax with at least one pharmaceutically acceptable excipient and at least one additional pharmaceutically acceptable excipient.

In an aspect, the present application provides pharmaceutical compositions comprising amorphous Venetoclax and at least one pharmaceutically acceptable excipient, in particular in the form of solid dispersions and adsorbates, and a process for preparing the same. In embodiments, the pharmaceutically acceptable excipient is selected from the excipients at any aspect of present application.

In embodiments, the present application provides adsorbates, wherein Venetoclax is associated with a suitable substrate. Suitable substrate may be a particulate and/or porous substrate, wherein this substrate has an outer and/or inner surface onto which the API may be adsorbed. This means that if the substrate has pores, these pores are filled by the Venetoclax and the substrate remains unaffected, it does not, at least not essentially, change during and/or after the adsorption. In embodiments, the suitable substrate is selected from the excipients at any aspect of present application.

Amorphous form of Venetoclax or its solid dispersion may be obtained alternatively either by employing a melt-extrusion technique or by combining a solution of Venetoclax as obtained any of the aspects of present application with a suitable anti-solvent. In embodiment, amorphous product may be obtained by employing suitable melt-extrusion conditions or any of the procedures known in the art for obtaining amorphous product by melt-extrusion technique. In embodiment, solution of Venetoclax may be combined with the anti-solvent at suitable temperature and for sufficient time to obtain amorphous product. Suitable anti-solvent is a solvent, wherein Venetoclax has low solubility and it may include, but not limited to aliphatic or cyclic ethers solvents, aliphatic or aromatic hydrocarbons or the like.

In an aspect, the present application provides acid addition salt of Venetoclax, wherein the acid may be selected from the group comprising of trifluoro acetic acid (TFA), oxalic acid, maleic acid, isethionic acid, ortho-phosphoric acid, citric acid, methanesulfonic acid and acetic acid.

In an embodiment, the acid addition salt of this aspect may contain Venetoclax and the acid in any stoichiometric ratio.

In an embodiment, the acid addition salt may be in crystalline or an amorphous form. In preferred embodiment, the acid addition salt may be in crystalline form.

In another aspect, the present application provides a trifluoro acetic acid (TFA) salt of venetoclax. In an embodiment, the trifluoro acetic acid (TFA) salt is a crystalline salt, characterized by a PXRD pattern of FIG. 12.

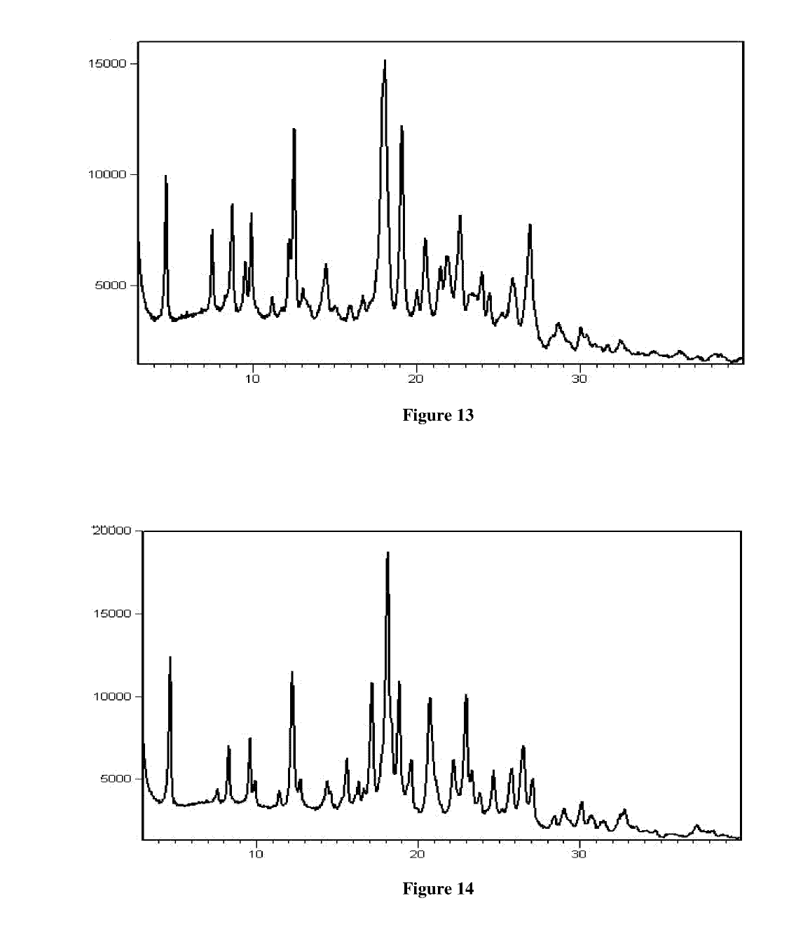

In another aspect, the present application provides an oxalic acid salt of Venetoclax. In an embodiment, the oxalic acid salt is a crystalline salt, characterized by a PXRD pattern of FIG. 13.

In another aspect, the present application provides a maleic acid salt of Venetoclax. In an embodiment, the Maleic acid salt is a crystalline salt, characterized by a PXRD pattern of FIG. 14.

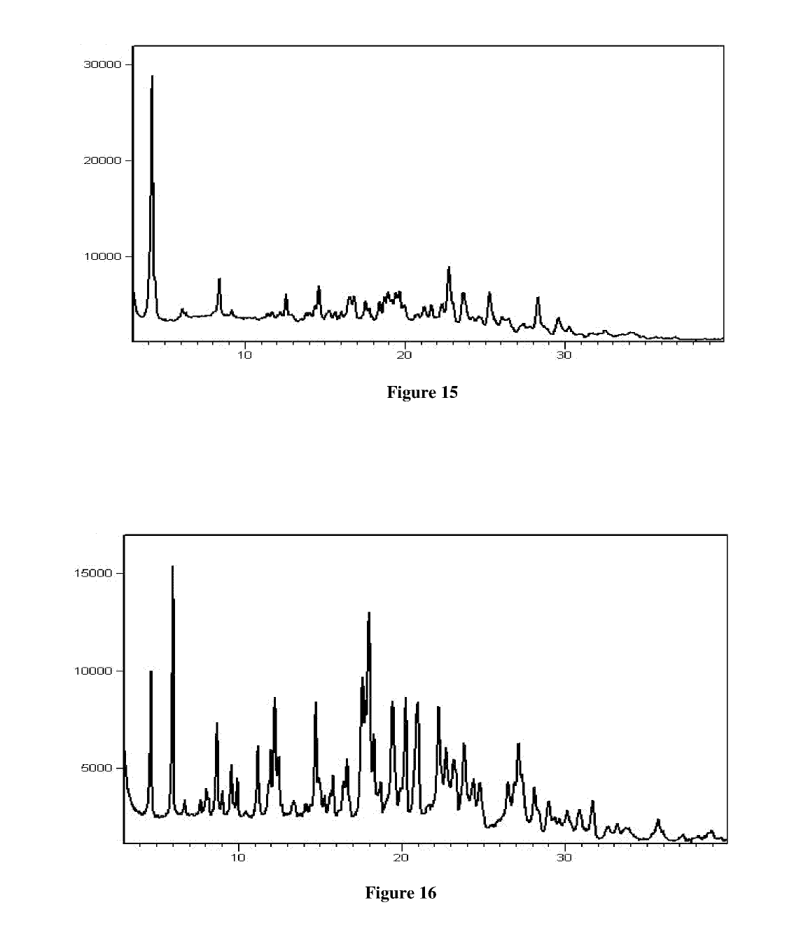

In another aspect, the present application provides an isethionic acid salt of Venetoclax. In an embodiment, the Isethionic acid salt is a crystalline salt, characterized by a PXRD pattern of FIG. 15.

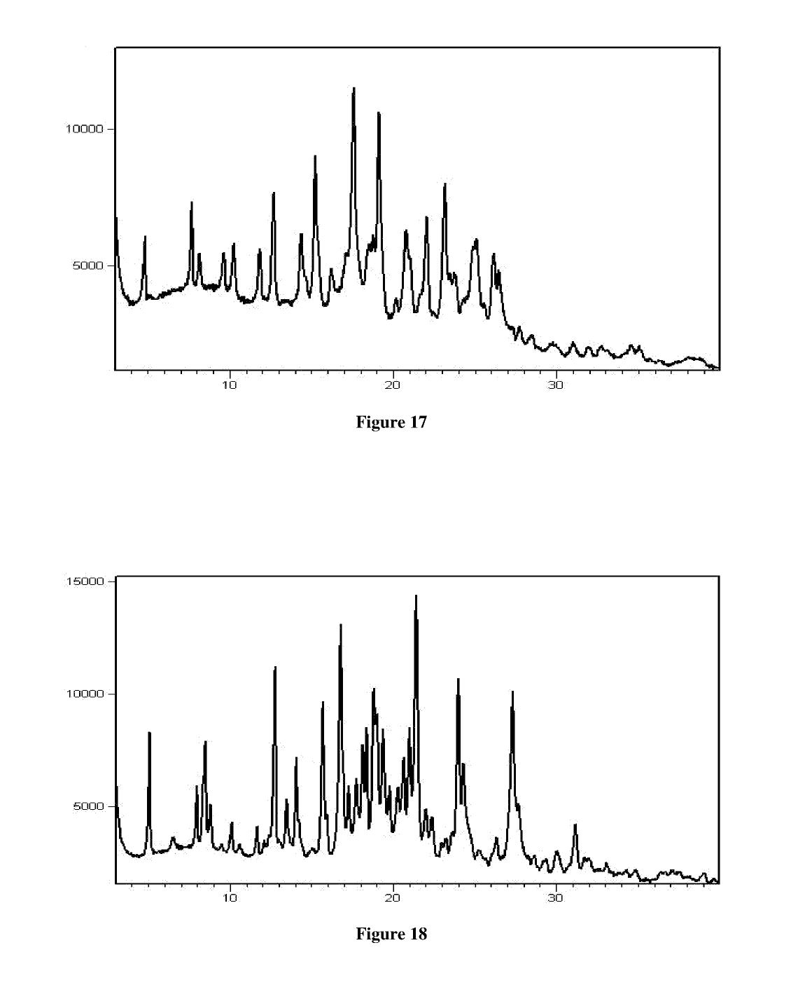

In another aspect, the present application provides an ortho-phosphoric salt of Venetoclax. In an embodiment, the ortho-phosphoric acid salt is a crystalline salt, characterized by a PXRD pattern of FIG. 17.

In another aspect, the present application provides a citric acid salt of Venetoclax. In an embodiment, the citric acid salt is a crystalline salt, characterized by a PXRD pattern of FIG. 18.

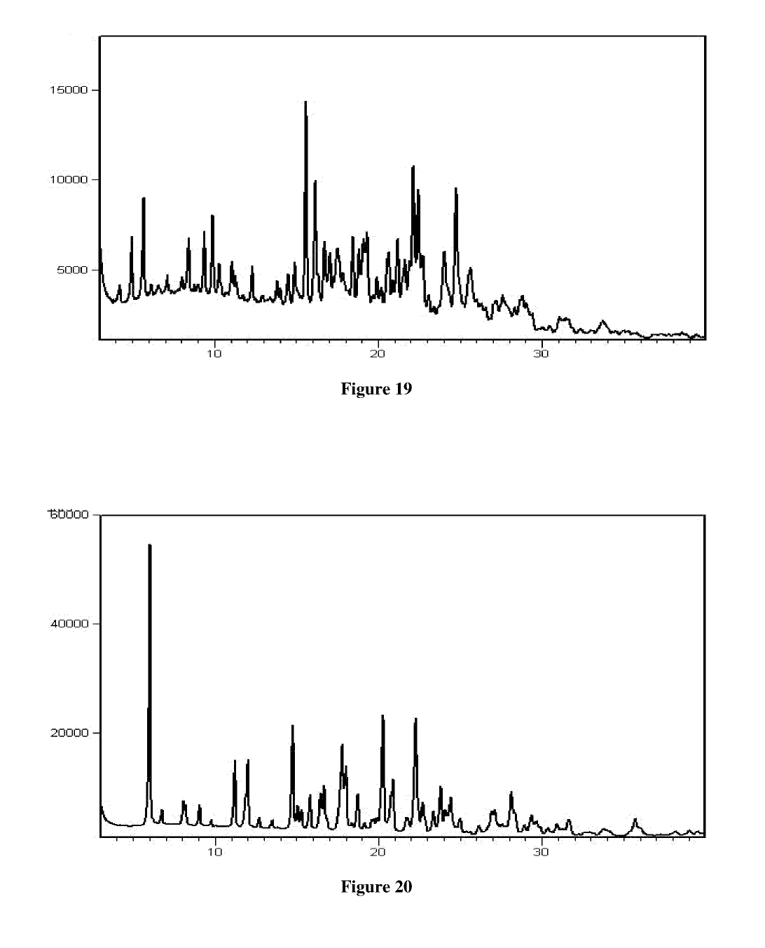

In another aspect, the present application provides a methanesulfonic acid salt of Venetoclax. In an embodiment, the methanesulfonic acid salt is a crystalline salt, characterized by a PXRD pattern of FIG. 19.

In another aspect, the present application provides an acetic acid salt of Venetoclax. In an embodiment, the acetic acid salt is a crystalline salt, characterized by a PXRD pattern of FIG. 20.

In another aspect, the present application provides a process for the preparation of salt of Venetoclax comprising the step of contacting an acid with Venetoclax, wherein acid may be selected from the group comprising of trifluoro acetic acid (TFA), oxalic acid, maleic acid, isethionic acid, ortho-phosphoric acid, citric acid, methanesulfonic acid and acetic acid.

In an embodiment Venetoclax may be contacted with an acid in a mole ratio of about 1:0.8 to 1:1.6.

In an embodiment, Venetoclax may be contacted with an acid in a heterogeneous or homogenous phase. In an embodiment, Venetoclax may be contacted with an acid in homogeneous phase. In an embodiment, solution comprising Venetoclax in an inert solvent may be contacted with an acid.

In an embodiment, the acid may be used either in concentrated or diluted form before contacting with Venetoclax.

In an embodiment, Venetoclax may be contacted with an acid at a suitable temperature at about 0° C. and above for time sufficient for salt formation. In an embodiment, the reaction mixture comprising Venetoclax and the acid may be stirred for sufficient time and at suitable temperature for the completion of salt formation.

In an embodiment, the reaction mixture comprising Venetoclax and the acid may be concentrated and/or cooled to suitable temperature before isolating the salt of Venetoclax.

In an embodiment, suitable anti-solvent may be added to the reaction mixture comprising Venetoclax and the acid before isolating the salt of Venetoclax.

Isolation of acid addition salt of Venetoclax may be carried out by any methods known in the art or procedures described in the present application. In an embodiment, acid addition salt of Venetoclax may be isolated by employing any of the techniques, but not limited to: decantation, filtration by gravity or suction, centrifugation, adding solvent to make slurry followed by filtration, or other techniques specific to the equipment used and the like, and optionally washing with a solvent.

In an embodiment, drying acid addition salt of Venetoclax may be carried out at temperatures and times sufficient to achieve desired quality of product. Drying may be carried out for any time period required for obtaining a desired quality, such as from about 15 minutes to 10 hours or longer.

In an alternate aspect, the present application provides a hydrochloride salt of Venetoclax. In an embodiment, the hydrochloride salt is a crystalline salt, characterized by a PXRD pattern of FIG. 16.

The present application provides a process for the preparation of hydrochloride salt of Venetoclax comprising the step of contacting a hydrochloric acid with Venetoclax. In an embodiment, 0.8 to 1.6. moles of hydrochloric acid may be contacted with Venetoclax may be used either in concentrated or diluted form before contacting with Venetoclax. In an embodiment, solution comprising Venetoclax in an inert solvent may be contacted with an acid. In an embodiment, the acid may be used either in concentrated or diluted form before contacting with Venetoclax.

In an embodiment, hydrochloride salt of Venetoclax may be obtained by any suitable method known in the art or process described or exemplified in the instant application for the preparation of hydrochloride salt or any other salt of Venetoclax.

It is worth noting, that the option of purifying a low soluble drug substances among BCS class II or class IV like Venetoclax, by conventional methods like recrystallization from a solvent or mixture of solvents may not be suitable due to the limited solvents. Therefore, purification of such drug substance through salt formation is a boon to a chemist. Venetoclax may be purified through the formation of a suitable salt followed by its neutralization to free from.

Further, these salts may be optionally purified by any method known in the art including recrystallization, before neutralization, unlike the free forms. The salt forms are generally regarded as superior in terms of solubility compared to respective free forms and may be conveniently recrystallized from suitable solvents according to techniques known in the art such cooling crystallization, anti-solvent addition, or the like.

The present application provides a purification process for Venetoclax, comprising the step of converting a salt of Venetoclax obtained according any of the previous aspects into its free form.

In another aspect, the present application provides a process for the preparation of Venetoclax, comprising the step of converting a salt of Venetoclax into its free form, wherein the salt may be selected from the group comprising of trifluoro acetic acid (TFA), oxalic acid, maleic acid, isethionic acid, hydrochloric acid, ortho-phosphoric acid, citric acid, methanesulfonic acid and acetic acid.

In an embodiment, the salt of Venetoclax may be converted to Venetoclax in free form by neutralization. In an embodiment, the salt may be neutralized in the presence of a suitable base.

Suitable base may include, but not limited to: either an inorganic base like hydroxides such as sodium hydroxide, potassium hydroxide, ammonium hydroxide; carbonates such sodium carbonate, potassium carbonate, ammonium carbonate; bicarbonates such as sodium bicarbonate, potassium bicarbonate, ammonium bicarbonate, or an organic base like amines such as triethyl amine, diisopropyl amine, diisopropyl ethyl amine; alkoxides such as methoxide, ethoxide, isopropoxide, tert. butoxide; N-heterocyclic Compounds; tetraalkylammonium and phosphonium hydroxides; amides; metal silanoates; and the like.

In another embodiment, the salt of Venetoclax may be converted to its free form by subjecting the acid addition salt to suitable conditions which may include, but not limited to: suspending the acid addition salt of Venetoclax in a suitable solvent optionally in the presence of a suitable base and optionally at elevated temperatures.

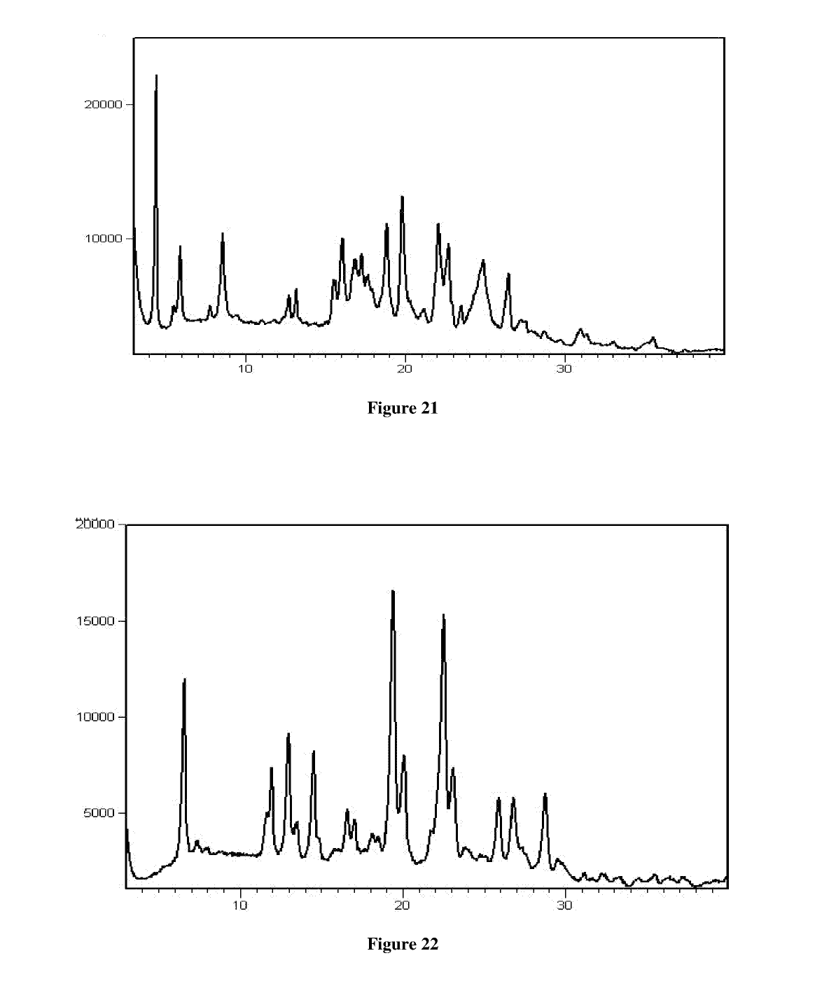

In another aspect, the present application provides a crystalline Form RT1 of Venetoclax characterized by a PXRD pattern comprising the peaks at about 4.39 and 8.56±0.2° 2θ. In an embodiment, the application provides crystalline Form RT1 of Venetoclax, characterized by a PXRD pattern having one or more additional peaks at about 5.91, 16.03, 22.08, 24.90 and 26.46±0.2° 2θ. In an embodiment, the application provides crystalline Form RT1 of Venetoclax, characterized by a PXRD pattern of FIG. 21.

In another aspect, the present application provides a crystalline Form RT2 of Venetoclax characterized by a PXRD pattern comprising the peaks at about 6.55, 19.37, 23.07, 26.82 and 28.70±0.2° 2θ. In an embodiment, the application provides crystalline Form RT2 of Venetoclax, characterized by a PXRD pattern having one or more-additional peaks at about 11.93, 12.95, 13.46, 14.49, 20.04, 22.50 and 25.86±0.2° 2θ. In an embodiment, the application provides crystalline Form RT2 of Venetoclax, characterized by a PXRD pattern of FIG. 22.

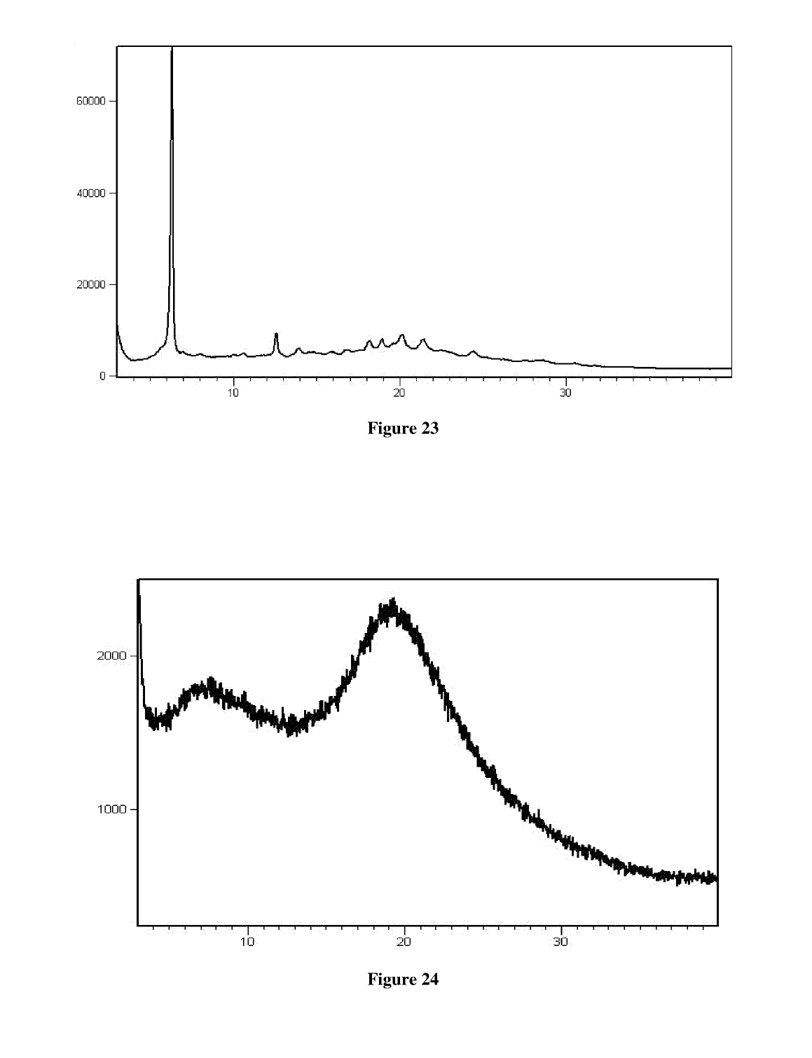

In another aspect, the present application provides a crystalline Form RT3 of Venetoclax characterized by a PXRD pattern comprising the peaks at about 6.30, 12.57 and 20.06±0.2° 2θ. In an embodiment, the application provides crystalline Form RT3 of Venetoclax, characterized by a PXRD pattern of FIG. 23.

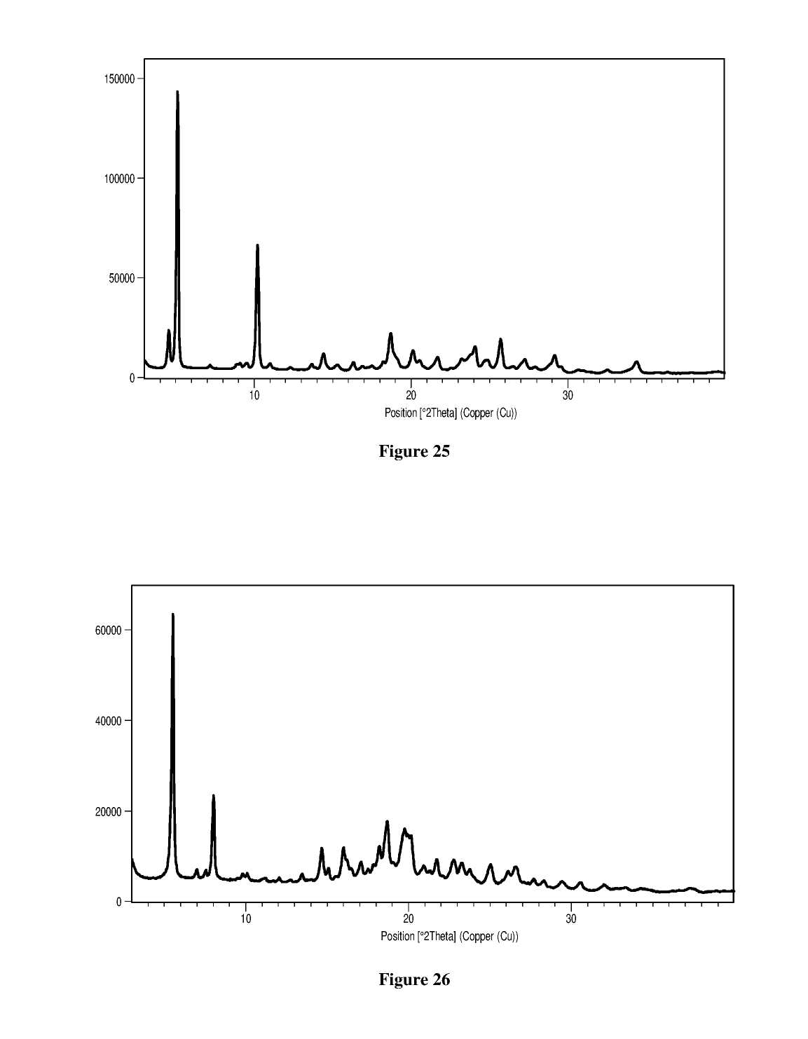

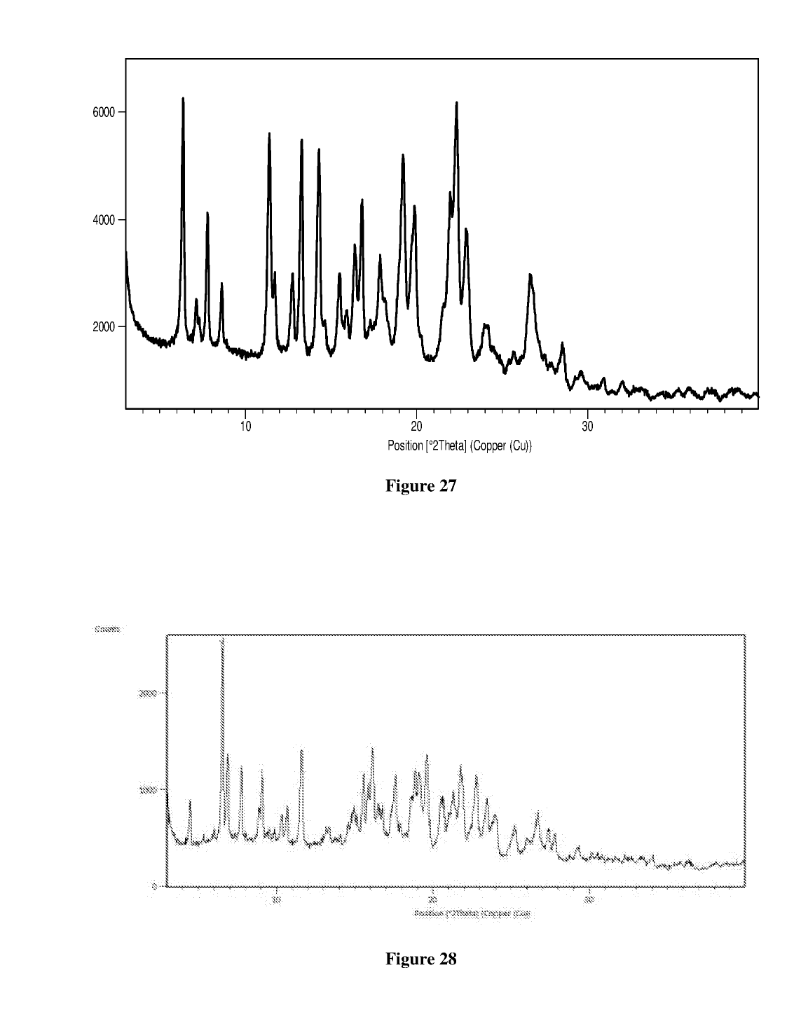

In another aspect, the present application provides a crystalline Form RT4 of Venetoclax characterized by a PXRD pattern comprising the peaks at about: 4.55 and 5.10±0.2° 2θ. In an embodiment, the application provides crystalline Form RT4 of Venetoclax, characterized by a PXRD pattern having one or more additional peaks at about 10.20, 18.67 and 25.67±0.2° 2θ. In an embodiment, the application provides crystalline Form RT4 of Venetoclax, characterized by a PXRD pattern of FIG. 25.

In another aspect, the present application provides a crystalline Form RT5 of Venetoclax characterized by a PXRD pattern comprising the peaks at about: 5.51 and 25.00±0.2° 2θ. In an embodiment, the application provides crystalline Form RT5 of Venetoclax, characterized by a PXRD pattern having one or more—additional peaks at about 8.00, 14.65, 16.00, 18.64 and 22.73±0.2° 2θ. In an embodiment, the application provides crystalline Form RT5 of Venetoclax, characterized by a PXRD pattern of FIG. 26.

In another aspect, the present application provides a process for the preparation of crystalline Form RT1 of Venetoclax, comprising the step of contacting Venetoclax with benzyl alcohol.

In an embodiment, contacting Venetoclax with benzyl alcohol may be carried out by suspending or dissolving Venetoclax in benzyl alcohol, optionally by heating. In an embodiment, Venetoclax may be dissolved in benzyl alcohol at suitable temperature of about 30° C. and above. Optionally, the solution may be filtered to make it particle free.

Alternatively, the solution may be provided by taking the reaction mixture containing Venetoclax in benzyl alcohol or a mixture thereof. Optionally, the solution may be filtered to make it particle free.

In an embodiment, the solution of Venetoclax in benzyl alcohol may be cooled to precipitate the solids to a suitable temperature and at which crystalline Form RT1 is formed and/or is stable.

In an embodiment, a solution of Venetoclax in benzyl alcohol may be optionally contacted with an anti-solvent. Anti-solvent is the solvent wherein Venetoclax or its crystalline Form RT1 has very low solubility or is insoluble. Anti-solvent may include, but not limited to hydrocarbons such as n-hexane, n-heptane, cyclohexane or the like; ethers such as diethyl ether, di isopropyl ether, methyl tert-Butyl ether or the like; any mixtures thereof.

In an embodiment, the anti-solvent may be contacted at suitable temperature for the nucleation of solids and for sufficient time for the formation of solids. The anti-solvent may be contacted in sufficient quantity to complete the formation of solids.

In an embodiment, the solution of Venetoclax in benzyl alcohol may be cooled to a suitable temperature before and/or after contacting with anti-solvent.

Isolation of crystalline Form RT1 of Venetoclax may be carried out by any methods known in the art or procedures described in the present application. In an embodiment, crystalline Form RT1 of Venetoclax may be isolated by employing any of the techniques, but not limited to: decantation, filtration by gravity or suction, centrifugation, adding solvent to make slurry followed by filtration, or other techniques specific to the equipment used and the like, and optionally washing with a solvent.

In an embodiment, drying crystalline Form RT1 of Venetoclax may be carried out at temperatures and times sufficient to achieve desired quality of product. Drying may be carried out for any time period required for obtaining a desired quality, such as from about 5 minutes to 10 hours or longer.

In another aspect, the present application provides a process for the preparation of crystalline Form RT2 of Venetoclax, comprising the steps of:
a) dissolving or suspending Venetoclax in methyl isopropyl ketone or a mixture thereof
b) optionally, contacting the solution of step a) with an anti-solvent
c) isolating crystalline Form RT2 of Venetoclax

In an embodiment, step a) may be carried out by dissolving or suspending Venetoclax in methyl isopropyl ketone or a mixture of methyl isopropyl ketone and any other solvent. Alternatively, the solution may be provided by taking the reaction mixture containing Venetoclax in methyl isopropyl ketone or a mixture thereof.

In an embodiment, the Venetoclax may be dissolved in methyl isopropyl ketone optionally by heating the mixture to obtain a homogenous solution. The solution may be filtered to make it particle free.

In an embodiment, the solution of Venetoclax in methyl isopropyl ketone may be cooled to precipitate the solids to a suitable temperature and at which crystalline Form RT2 is formed and/or is stable.

In an embodiment, optionally the solution of step a) may be contacted with an anti-solvent. Anti-solvent may include, but not limited to hydrocarbons such as n-hexane, n-heptane, cyclohexane or the like; ethers such as diethyl ether, di isopropyl ether, methyl tert-butyl ether or the like; water; or any mixtures thereof.

In an embodiment, the anti-solvent may be contacted at suitable temperature for the nucleation of solids and for sufficient time for the formation of solids. The anti-solvent may be contacted in sufficient quantity to complete the formation of solids.

Isolation of crystalline Form RT2 of Venetoclax may be carried out by any methods known in the art or procedures described in the present application. In an embodiment, crystalline Form RT2 of Venetoclax may be isolated by employing any of the techniques, but not limited to: decantation, filtration by gravity or suction, centrifugation, adding solvent to make slurry followed by filtration, or other techniques specific to the equipment used and the like, and optionally washing with a solvent.

In an embodiment, drying crystalline Form RT2 of Venetoclax may be carried out at temperatures and times sufficient to achieve desired quality of product. Drying may be carried out for any time period required for obtaining a desired quality, such as from about 5 minutes to 10 hours or longer.

In another aspect, the present application provides a process for the preparation of crystalline Form RT2 of Venetoclax, comprising the steps of:
a) dissolving or suspending Venetoclax in methyl-tertiary butyl ether or a mixture thereof
b) optionally, contacting the solution of step a) with an anti-solvent
c) isolating crystalline Form RT2 of Venetoclax

In an embodiment, step a) may be carried out by dissolving or suspending Venetoclax in methyl-tertiary butyl ether or a mixture of methyl-tertiary butyl ether and any other solvent. Alternatively, the solution or suspension may be provided by taking the reaction mixture containing Venetoclax in methyl-tertiary butyl ether or a mixture thereof.

In an embodiment, the Venetoclax may be suspended in Methyl-Tertiary Butyl Ether at suitable temperature of about 0° C. to reflux temperature. In an embodiment, the Venetoclax may be suspended in methyl-tertiary butyl ether for sufficient time to complete the formation of crystalline form RT2 of about one hour or more.

In an embodiment, the Venetoclax may be dissolved in methyl-tertiary butyl ether optionally by heating the mixture to obtain a homogenous solution. The solution may be filtered to make it particle free.

In an embodiment, the solution of Venetoclax in methyl-tertiary butyl ether may be cooled to precipitate the solids to a suitable temperature at which crystalline Form RT2 is formed and/or is stable.

In an embodiment, optionally the solution of Venetoclax in methyl-tertiary butyl ether may be contacted with an anti-solvent. Anti-solvent may include, but not limited to hydrocarbons such as n-hexane, n-heptane, cyclohexane or the like; water; or any mixtures thereof.

In an embodiment, the anti-solvent may be contacted at suitable temperature for the nucleation of solids and for sufficient time for the formation of solids. The anti-solvent may be contacted in sufficient quantity to complete the formation of solids.

Isolation of crystalline Form RT2 of Venetoclax may be carried out by any methods known in the art or procedures described in the present application. In an embodiment, crystalline Form RT2 of Venetoclax may be isolated by employing any of the techniques, but not limited to: decantation, filtration by gravity or suction, centrifugation, adding solvent to make slurry followed by filtration, or other techniques specific to the equipment used and the like, and optionally washing with a solvent.

In an embodiment, drying crystalline Form RT2 of Venetoclax may be carried out at temperatures and times sufficient to achieve desired quality of product. Drying may be carried out at about 30° C. or above at which crystalline form RT2 is stable and for any time period required for obtaining a desired quality, such as from about 5 minutes to 10 hours or longer. Crystalline form RT2 is stable for 3 months period or longer.

In another aspect, the present application provides a process for the preparation of crystalline Form RT3 of Venetoclax, comprising the steps of:
a) providing a solution of Venetoclax in a methylene chloride or a mixture thereof
b) optionally washing the solution of step a) with water
c) removing the solvent of step a) to obtain crystalline Form RT3 of Venetoclax.

In an embodiment, the solution of Venetoclax of step a) may be provided by dissolving Venetoclax in methylene chloride or a mixture thereof, optionally by heating. Alternatively, the solution may be provided by taking the reaction mixture containing Venetoclax in methylene chloride or a mixture thereof. The solution may be filtered to make it particle free.

In an embodiment, the solution of step a) may be optionally washed with water at a suitable temperature. The water washings may be repeated to attain the desired quality of the product. The washed aqueous layer may be extracted back with methylene chloride, if required and combined with the solution of step a).

In an embodiment, the combined solution of Venetoclax in a methylene chloride may be dried over suitable drying agent such as sodium sulfate or the like to remove the residual traces of water.

In an embodiment, the solution of step a) or b) may be cooled to suitable temperature before the removal of the solvent at step c). In an embodiment, step c) may be carried out by removing the solvent of step a) or b) to obtain crystalline Form RT3 of Venetoclax. Removal of the solvent may be carried out at suitable temperature from freezing point to boiling point of the methylene chloride or mixture thereof.

The solvent of step a) or b) may be removed using suitable techniques known in the art or procedures described or exemplified in the present application. Suitable techniques for the removal of the solvent may include but not limited to evaporation of solvent under atmospheric pressure or reduced pressure; spray drying; sublimation such as freeze drying or lyophilisation; thin film drying such as drying in agitated thin film drier; or the like.

In an embodiment, the solvent may be removed by evaporation under reduced pressure at about 0° C. to boiling point of the solvent or mixture thereof.

Isolation of crystalline Form RT3 of Venetoclax may be carried out by any methods known in the art or procedures described in the present application. In an embodiment, crystalline Form RT3 of Venetoclax may be isolated by employing any of the techniques, but not limited to: scratching the walls of the container with a spatula, adding solvent to make slurry followed by filtration, decantation, filtration by gravity or suction, centrifugation, or other techniques specific to the equipment used and the like, and optionally washing with a solvent.

In an embodiment, drying crystalline Form RT3 of Venetoclax may be carried out at temperatures and times sufficient to achieve desired quality of product. Drying may be carried out for any time period required for obtaining a desired quality, such as from about 5 minutes to 10 hours or longer.

In another aspect, the present application provides a process for the preparation of crystalline Form RT4 of Venetoclax, comprising the steps of
a) dissolving or suspending Venetoclax in Methyl Iso-Butyl Ketone or a mixture thereof
b) optionally, contacting the solution of step a) with an anti-solvent
c) isolating crystalline Form RT4 of Venetoclax

In an embodiment, step a) may be carried out by dissolving or suspending Venetoclax in methyl iso-butyl ketone or a mixture of methyl iso-butyl ketone and any other solvent. Alternatively, the solution or suspension may be provided by taking the reaction mixture containing Venetoclax in methyl iso-butyl ketone or a mixture thereof.

In an embodiment, the Venetoclax may be suspended in Methyl Iso-Butyl Ketone at suitable temperature of about 0° C. to reflux temperature. In an embodiment, the Venetoclax may be suspended in methyl iso-butyl ketone for sufficient time to complete the formation of crystalline form RT4 of about one hour or more.

In an embodiment, the Venetoclax may be dissolved in methyl iso-butyl ketone optionally by heating the mixture to obtain a homogenous solution. The solution may be filtered to make it particle free.

In an embodiment, the solution of Venetoclax in methyl iso-butyl ketone may be cooled to precipitate the solids to a suitable temperature at which crystalline Form RT4 is formed and/or is stable.

In an embodiment, optionally the solution of Venetoclax in methyl iso-butyl ketone may be contacted with an anti-solvent. Anti-solvent may include, but not limited to hydrocarbons such as n-hexane, n-heptane, cyclohexane or the like; water; or any mixtures thereof.

In an embodiment, the anti-solvent may be contacted at suitable temperature for the nucleation of solids and for sufficient time for the formation of solids. The anti-solvent may be contacted in sufficient quantity to complete the formation of solids.

Isolation of crystalline Form RT4 of Venetoclax may be carried out by any methods known in the art or procedures described in the present application. In an embodiment, crystalline Form RT4 of Venetoclax may be isolated by employing any of the techniques, but not limited to: decantation, filtration by gravity or suction, centrifugation, adding solvent to make slurry followed by filtration, or other techniques specific to the equipment used and the like, and optionally washing with a solvent.

In an embodiment, drying crystalline Form RT4 of Venetoclax may be carried out at temperatures and times sufficient to achieve desired quality of product. Drying may be carried out at about 30° C. or above at which crystalline form RT4 is stable and for any time period required for obtaining a desired quality, such as from about 5 minutes to 10 hours or longer.

In another aspect, the present application provides a process for the preparation of crystalline Form RT5 of Venetoclax, comprising the steps of:
a) dissolving or suspending Venetoclax in 1,4-dioxane or a mixture thereof.
b) optionally, contacting the solution of step a) with an anti-solvent
c) isolating crystalline Form RT5 of Venetoclax.

In an embodiment, step a) may be carried out by dissolving or suspending Venetoclax in 1,4-dioxane or a mixture thereof. Alternatively, the solution or suspension may be provided by taking the reaction mixture containing Venetoclax in 1,4-dioxane or a mixture thereof.

In an embodiment, the Venetoclax may be suspended in 1,4-dioxane or a mixture thereof at suitable temperature of about 0° C. to reflux temperature. In an embodiment, the Venetoclax may be suspended in 1,4-dioxane or a mixture thereof for sufficient time to complete the formation of crystalline form RT5 of about one hour or more.

In an embodiment, the Venetoclax may be dissolved in 1,4-dioxane or a mixture thereof optionally by heating the mixture to obtain a homogenous solution. The solution may be filtered to make it particle free.

In an embodiment, the solution of Venetoclax in 1,4-dioxane or a mixture thereof may be cooled to precipitate the solids to a suitable temperature at which crystalline Form RT5 is formed and/or is stable.

In an embodiment, optionally the solution of Venetoclax in 1,4-dioxane or a mixture thereof may be contacted with an anti-solvent. Anti-solvent may include, but not limited to hydrocarbons such as n-hexane, n-heptane, cyclohexane or the like; water; or any mixtures thereof.

In an embodiment, the anti-solvent may be contacted at suitable temperature for the nucleation of solids and for sufficient time for the formation of solids. The anti-solvent may be contacted in sufficient quantity to complete the formation of solids.

Isolation of crystalline Form RT5 of Venetoclax may be carried out by any methods known in the art or procedures described in the present application. In an embodiment, crystalline Form RT5 of Venetoclax may be isolated by employing any of the techniques, but not limited to: decantation, filtration by gravity or suction, centrifugation, adding solvent to make slurry followed by filtration, or other techniques specific to the equipment used and the like, and optionally washing with a solvent.

In an embodiment, drying crystalline Form RT5 of Venetoclax may be carried out at temperatures and times sufficient to achieve desired quality of product. Drying may be carried out at about 30° C. or above at which crystalline form RT5 is stable and for any time period required for obtaining a desired quality, such as from about 5 minutes to 10 hours or longer.

Starting materials used for the preparation of crystalline Form RT1, Form RT2, Form RT3, Form RT4 and Form RT5 of Venetoclax or acid addition salts thereof according to any of the aspects of the present application may be any crystalline or amorphous in nature. Further, these starting materials may be purified according to any of the method known in the art such as recrystallization, slurrying, acid-base treatment i.e., salt making and breaking, chromatography, fractional distillation or any other separation methods, before using.

In another aspect, the present application provides a pharmaceutical composition comprising the acid addition salts of Venetoclax or solid forms thereof and at least one additional pharmaceutically acceptable excipient, wherein the acid may be selected from the group comprising of trifluoro acetic acid (TFA), oxalic acid, maleic acid, isethionic acid, hydrochloric acid, ortho-phosphoric acid, citric acid, methanesulfonic acid and acetic acid.

Similar procedures for the preparation of any Venetoclax salts described here may be useful to produce other salts of Venetoclax comprising the acid addition salts such as adipate, alginate, bicarbonate, aspartate, benzoate, benzenesulfonate, bisulfate, butyrate, camphorate, camphorsulfonate, digluconate, formate, fumarate, glycerophosphate, glutamate, hemisulfate, heptanoate, hexanoate, hydrobromide, hydroiodide, lactobionate, lactate, mesitylenesulfonate, naphthylenesulfonate, nicotinate, pamoate, pectinate, persulfate, picrate, propionate, succinate, tartrate, thiocyanate, trichloroacetic, para-toluenesulfonate, and undecanoate; basic addition salts such as hydroxide, carbonate or bicarbonate of cations such as lithium, sodium, potassium, calcium, and magnesium.

Aspects of the present application provide alternative processes for the preparation of Venetoclax and intermediates thereof.

In an aspect, the present application provides a process for the preparation of Venetoclax, comprising the steps of
a) hydrolysis of the cyano compound of formula (II) to obtain corresponding carboxylic acid or its ester of formula (III);

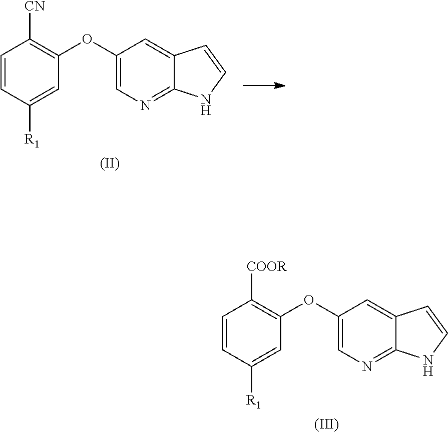

b) converting the carboxylic acid of formula (III) into Venetoclax.
   Wherein R may be selected from the group comprising of hydrogen, alkyl, aryl, arylalkyl or heteroaryl; R₁ may be selected from the group comprising of leaving group such as a halogen, optionally protected piperazine or a group of formula (A).

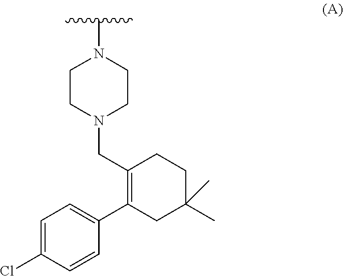 [SMILES: *N1CCN(CC2=C(c3ccc(Cl)cc3)CC(C)(C)CC2)CC1]

In an embodiment, a process for the preparation of Venetoclax, comprising the steps of
a) hydrolysis of the cyano compound of formula (IIa) to obtain corresponding carboxylic acid or its ester of formula (IIIa), wherein X is any halogen such as fluorine, chlorine, bromine or iodine;

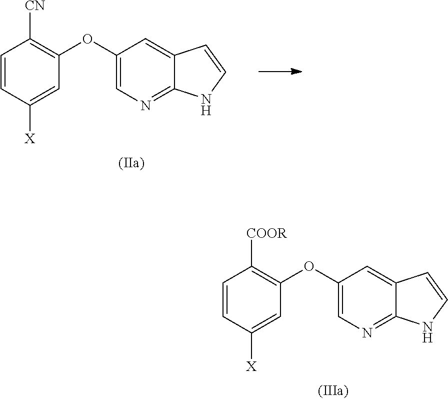

b) converting the carboxylic acid of formula (IIIa) of step a) to Venetoclax.

In another embodiment, a process for the preparation of Venetoclax, comprising the steps of
a) hydrolysis of the cyano compound of formula (IIb) to obtain corresponding carboxylic acid or its ester of formula (IIIb), wherein P is hydrogen or any nitrogen protecting group such as BoC or Cbz;

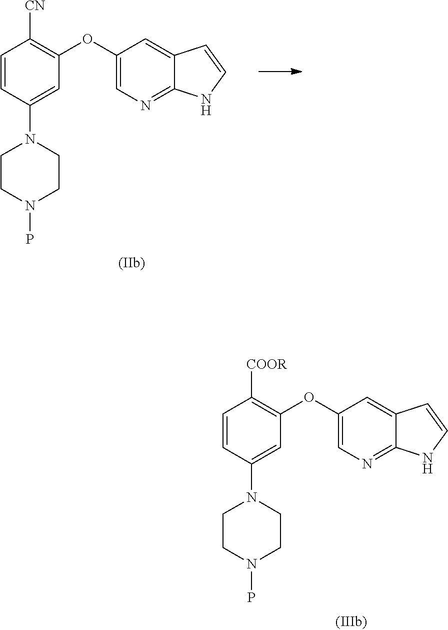

b) converting the carboxylic acid of formula (IIIb) of step a) to Venetoclax.

In yet another embodiment, a process for the preparation of Venetoclax, comprising the steps of
a) hydrolysis of the cyano compound of formula (IIc) to obtain corresponding carboxylic acid or its ester of formula (IIIc);

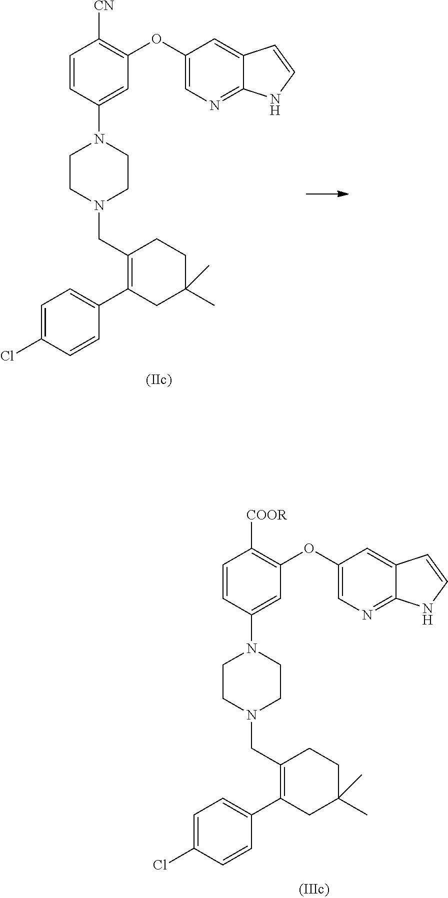

b) converting the carboxylic acid of formula (IIIc) of step a) to Venetoclax.

In an embodiment, the hydrolysis of step a) of this aspect may be carried out in the presence of a suitable base or acid. Base may include, but not limited to hydroxides such as sodium hydroxide, potassium hydroxide, magnesium hydroxide, Lithium hydroxide; alkoxides such as sodium or potassium tert butoxide or the like. Acid may include, but not limited to concentrated or diluted forms of sulfuric acid, nitric acid, acetic acid or hydrogen halides such as hydrochloride, hydrobromide, hydroiodide or the like.

In an embodiment, the hydrolysis of step a) of this aspect may be carried out in the presence of an inert solvent such as water, methanol, ethanol, 2- or 1-propanol, 1- or 2-butanol, ethyl acetate, isopropyl acetate, acetone, dichloromethane, 1,4-dioxane, ethylene glycol, diethylene glycol, N,N dimethyl formamide, tetrahydrofuran or mixtures thereof.

In an embodiment, the hydrolysis of step a) of this aspect may be carried out at a suitable temperature of 0° C. to reflux temperature of the solvent used. The reaction may be carried out for sufficient time till the completion of hydrolysis for at least 1 hour or more.

Step b) of this aspect involves the conversion of carboxylic acid of formula (III) into Venetoclax. Conversion may be carried out by any method known in the art or according to procedures described or exemplified in any aspect of the present application.

In an embodiment, step b) may be carried out by reacting carboxylic acid of formula (III) with an 3-nitro-4-(((tetrahydro-2H-pyran-4-yl)methyl)amino)benzene sulfonamide to obtain a compound of formula (V) and/or Venetoclax as depicted in following scheme-3.

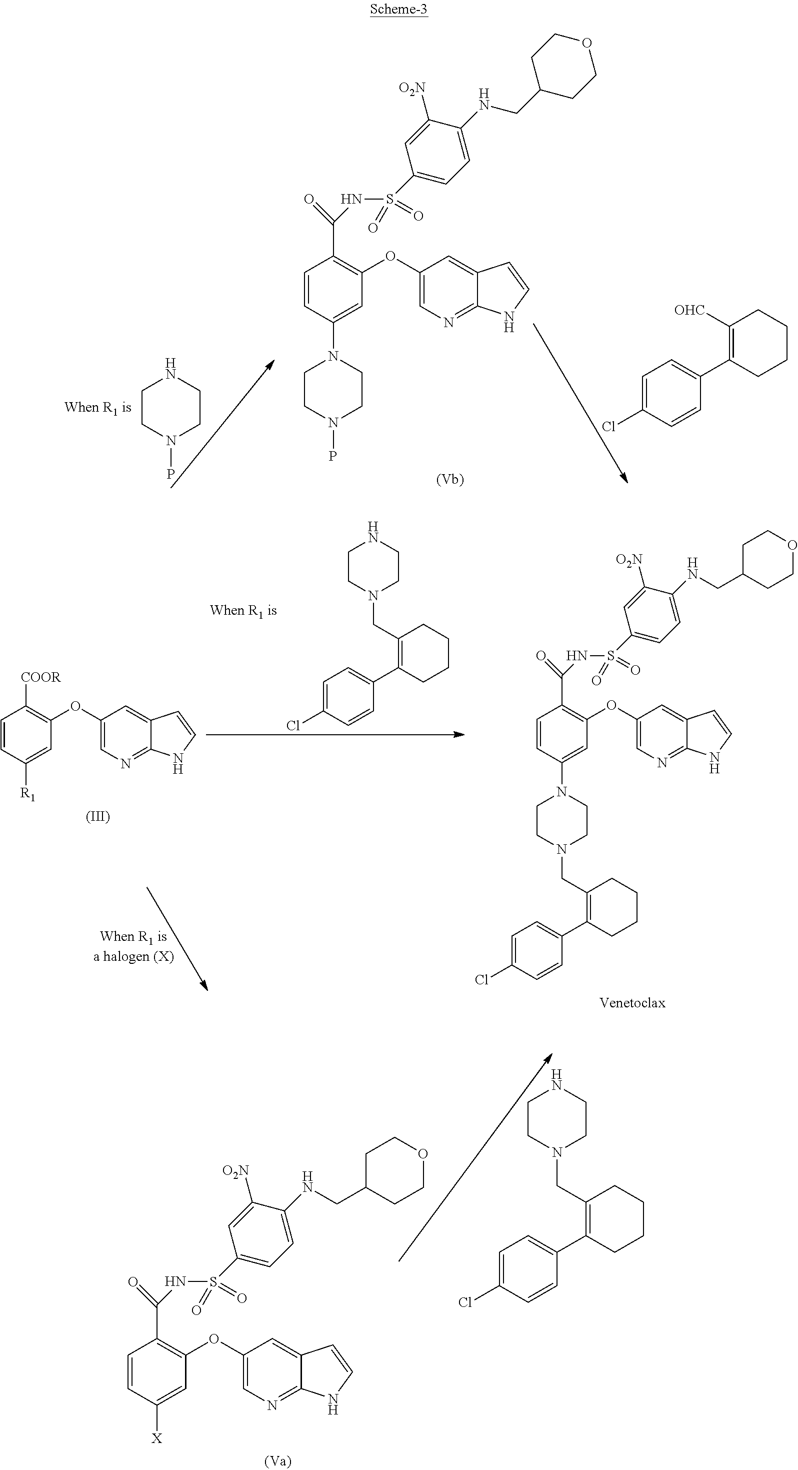

In an alternate aspect, the present application provides a process for the preparation of Venetoclax, comprising the steps of
a) hydrolysis of the cyano compound of formula (II) to obtain corresponding amide of formula (X);

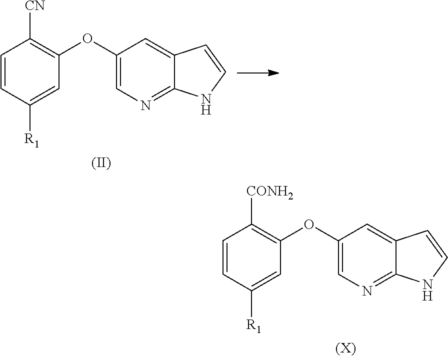

b) converting the amide of formula (X) into Venetoclax.
   Wherein R₁ may be selected from the group comprising of leaving group such as a halogen, optionally protected piperazine or a group of formula (A).

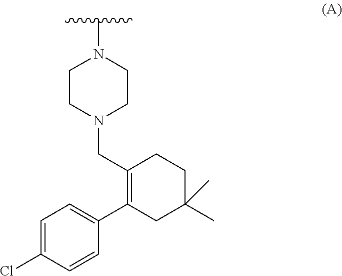 [SMILES: *N1CCN(CC2=C(c3ccc(Cl)cc3)CC(C)(C)CC2)CC1]

In an embodiment, hydrolysis of the cyano compound of formula (II) to obtain corresponding amide of formula (X) may be carried out according methods known in the art. In an embodiment, hydrolysis of the cyano compound of formula (II) may be carried out under suitable conditions such as acidic or basic conditions.

Step b) of this aspect involves the conversion of amide of formula (X) into Venetoclax. Conversion may be carried out by any method known in the art or according to procedures described or exemplified in any aspect of the present application.

In an embodiment, step b) may be carried out by reacting amide of formula (X) with an 3-nitro-4-(((tetrahydro-2H-pyran-4-yl)methyl)amino)benzene sulfonyl chloride to obtain a compound of formula V) and/or Venetoclax.

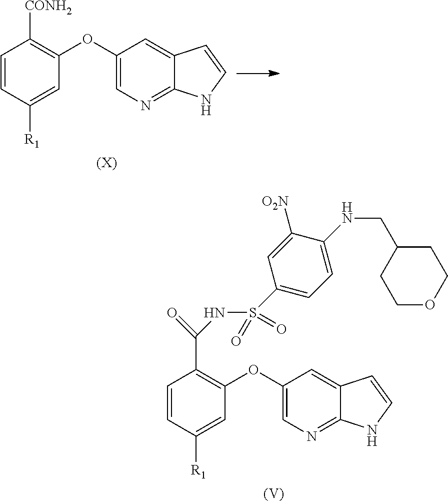

Amide of formula (X) may be reacted with an 3-nitro-4-(((tetrahydro-2H-pyran-4-yl)methyl)amino)benzene sulfonyl chloride under suitable conditions known in the art or according to the procedures described or exemplified in the instant application.

In an alternate embodiment, the step b) may be carried out by hydrolysing the amide of formula (X) to corresponding carboxylic acid or ester thereof of formula (III) under suitable conditions followed by its reaction with 3-nitro-4-(((tetrahydro-2H-pyran-4-yl)methyl)amino)benzene sulfonamide as described in the previous aspect. Hydrolysis of amide of formula (X) may be carried out under suitable conditions known in the art or according to the procedures described or exemplified in the instant application.

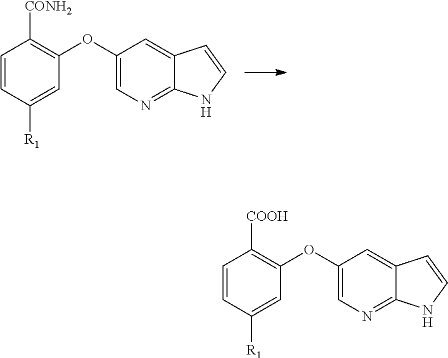

In another aspect, the present application provides a process for the preparation of Venetoclax, comprising the step of reacting the compound of formula (III) with 3-nitro-4-(((tetrahydro-2H-pyran-4-yl)methyl)amino)benzene sulfonamide to obtain a compound of formula (V).

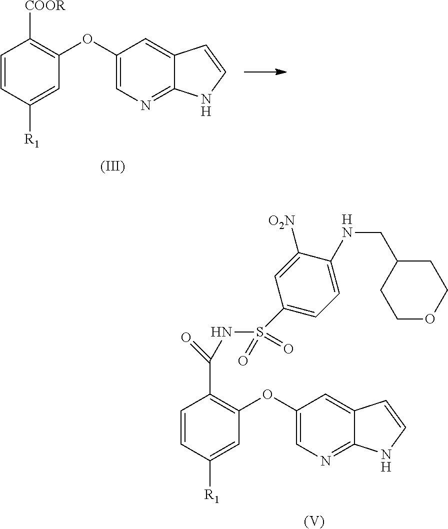

wherein R₁ may be selected from the group comprising of leaving group such as a halogen or an optionally protected piperazine; R may be selected from the group comprising of hydrogen, alkyl, aryl, arylalkyl or heteroaryl.

In an embodiment, a process for the preparation of Venetoclax comprising the step of reacting the compound of formula (IIIa) with 3-nitro-4-(((tetrahydro-2H-pyran-4-yl)methyl)amino)benzene sulfonamide in the presence of an inert solvent to obtain compound of formula (Va).

In an embodiment, a process for the preparation of Venetoclax comprising the step of reacting the compound of formula (IIIb) with 3-nitro-4-(((tetrahydro-2H-pyran-4-yl)methyl)amino)benzene sulfonamide to obtain compound of formula (Vb).

In an embodiment, the process of this aspect further comprises the step of converting the compounds of formula (Va) and (Vb) to Venetoclax by any of the methods known in the art or according to the procedures described or exemplified in any aspect of the instant application.

In an embodiment, the compound of formula (III) may be reacted with 3-nitro-4-(((tetrahydro-2H-pyran-4-yl)methyl)amino)benzene sulfonamide in the presence of suitable coupling agent.

The suitable coupling agent includes, but are not limited to N,N′-Dicyclohexylcarbodiimide (DCC), N, N′-diisopropylcarbodiimide (DIC), N-(3-dimethylaminopropyl)-N′-ethyl-carbodiimide (EDC) or a salt thereof, O-(benzotriazol-1-yl)-N,N,N′,N′-tetramethyluronium-tetrafluoroborate (TBTU), O-(benzotriazol-1-yl)-N,N,N′,N′-tetramethyluronium-hexafluorophosphate (HBTU), O-(7-azabenzotriazol-1-yl)-N,N,N′,N′-tetramethyluronium-hexafluorophosphate (HATU), (benzothazol-1-yloxy)-tris-(dimethylamino)-phosphonium-hexafluoro-phosphate (BOP), (benzothazol-1-yloxy)-thpyrrolidinophosphonium-hexafluorophosphate (PyBOP), cyanuric chloride, 2-chloro-4,6-dimethoxy-1,3,5-triazine (CDMT), 4-(4,6-dimethoxy-1,3,5-triazin-2-yl)-4-methyl morpholinium chloride (DMTMM) or the like or any mixture thereof.

In embodiment, optionally the reaction may be carried out in the presence of a suitable catalyst. The suitable catalysts include but are not limited to N-hydroxysuccinimide (HOSu), N-hydroxy-5-norbornene-2,3-dicarboximide (HONB), 1-hydroxybenzotriazole (HOBt), 6-chloro-1-hydroxybenzotriazole (6-C1-HOBt), 1-hydroxy-7-azabenzotriazole (HOAt), 3-hydroxy-4-oxo-3,4-dihydro-1,2,3-benzotriazine (HODhbt) and its aza derivative (HODhat), or a base like pyridine, dimethyl aminopyridine, diisopropyl amine, diisopropyl ethyl amine, triethyl amine; or the like.

In an embodiment, process of this aspect may be carried out at a suitable temperature of 0° C. to reflux temperature of the solvent used. The reaction may be carried out for sufficient time till the completion of reaction for at least 1 hour or more.

In another aspect, the present application provides a process for the preparation of cyano compound of formula (IIa) comprising the step of reacting 2-halo benzonitrile of formula (VIIa) with optionally protected 1H-pyrrolo[2,3-b]pyridin-5-ol or its reactive derivative; wherein X may be any halogen such as fluorine, chlorine, bromine or iodine and P may be hydrogen or any nitrogen protecting group such as BoC or Cbz.

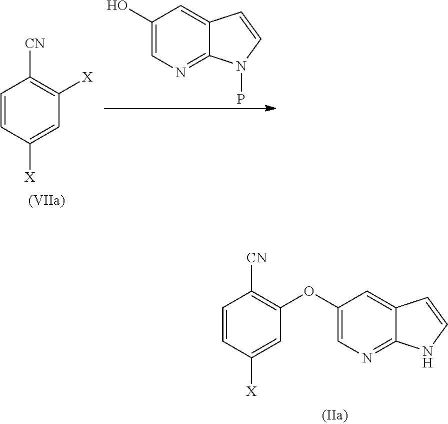

In an embodiment, reaction between 2-halo benzonitrile of formula (VIIa) with optionally protected 1H-pyrrolo[2,3-b]pyridin-5-ol or its reactive derivative in may be carried out in the presence of a suitable base. Suitable base may include, but not limited to hydroxides such as sodium hydroxide, potassium hydroxides; carbonates such as sodium carbonate, potassium carbonates, cesium carbonate; alkoxides such as sodium or potassium tert. butoxide; dibasic or tribasic phosphates such as potassium phosphate or dipotassium hydrogen phosphate; or the like.

In an embodiment, cyano compound of formula (IIa) may be prepared by reacting 2,4-dihalo benzonitrile of formula (VIIa) with optionally protected 1H-pyrrolo[2,3-b]pyridin-5-ol or its reactive derivative in the presence of an inert solvent system comprising at least one solvent selected from 1,4-dioxane, diglyme, dimethylsulphoxide, THF, toluene, or the like. In preferred embodiment, inert solvent system comprising 1,4-dioxane.

In an embodiment, reaction between 2,4-dihalo benzonitrile of formula (VIIa) with optionally protected 1H-pyrrolo[2,3-b]pyridin-5-ol or its reactive derivative may be carried out optionally in the presence of suitable phase transfer catalyst.

In an embodiment, unprotected cyano compound of formula (IIa) (wherein P is Hydrogen) may be obtained by deprotection the compound (IIa) (wherein P is a nitrogen protecting group).

In an embodiment, process of this aspect may be carried out at a suitable temperature of 0° C. to reflux temperature of the solvent used. The reaction may be carried out for sufficient time till the completion of reaction for at least 1 hour or more.

In another aspect, the present application provides a process for the preparation of cyano compounds of formula (IIb) comprising the step of
a) reacting 2-halo benzonitrile of formula (VIIb) with optionally protected 1H-pyrrolo[2,3-b]pyridin-5-ol or its reactive derivative (or)
b) reacting a compound of formula (IIa) with optionally protected piperazine.

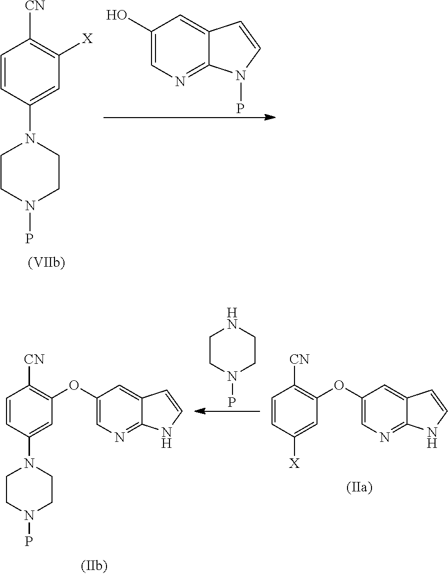

In an embodiment, step a) of this aspect may be carried out by reacting 2-halo benzonitrile of formula (VIIb) with optionally protected 1H-pyrrolo[2,3-b]pyridin-5-ol or its reactive derivative by any method known in the art or according to procedure described or exemplified in any aspect of the present application.

In an embodiment, 2-halo benzonitrile of formula (VIIb) may be reacted with optionally protected 1H-pyrrolo[2,3-b]pyridin-5-ol or its reactive derivative in the presence of a suitable base, an inert solvent and optionally in the presence of a phase transfer catalyst. In an embodiment, reaction may be carried out in the presence of a solvent system comprising at least one solvent selected from 1,4-dioxane, diglyme, dimethylsulphoxide, THF, toluene, or the like. In preferred embodiment, inert solvent system comprising 1,4-dioxane.

In alternate embodiment, step a) of this aspect may be carried out by reacting a compound of formula (IIa) with optionally protected piperazine by any method known in the art or according to procedure described or exemplified in any aspect of the present application.

In an embodiment, reaction of the compound of formula (IIa) with optionally protected piperazine may be carried out in the presence of a suitable base, an inert solvent and optionally in the presence of a phase transfer catalyst.

Suitable base may include, but not limited to hydroxides such as sodium hydroxide, potassium hydroxides; carbonates such as sodium carbonate, potassium carbonates, cesium carbonate; alkoxides such as sodium or potassium tert. butoxide; dibasic or tribasic phosphates such as potassium phosphate or dipotassium hydrogen phosphate; or the like.

In an embodiment, unprotected cyano compound of formula (IIb) (wherein P is Hydrogen) may be obtained by deprotection the compound (IIb) (wherein P is a nitrogen protecting group).

In embodiments of this aspect, process of this aspect may be carried out at a suitable temperature of 0° C. to reflux temperature of the solvent used. The reaction may be carried out for sufficient time till the completion of reaction for at least 1 hour or more.

In another aspect, the present application provides a process for the preparation of cyano compounds of formula (IIc) comprising the step of
a) reacting 2-halo benzonitrile of formula (VIIc) with optionally protected 1H-pyrrolo[2,3-b]pyridin-5-ol or its reactive derivative (or)
b) reacting a compound of formula (IIb) with 4′-chloro-5,5-dimethyl-3,4,5,6-tetrahydro-[1,1′-biphenyl]-2-carbaldehyde (or)
c) reacting a compound of formula (IIa) with 1-((4′-chloro-5,5-dimethyl-3,4,5,6-tetrahydro-[1,1′-biphenyl]-2-yl)methyl)piperazine, as depicted below.

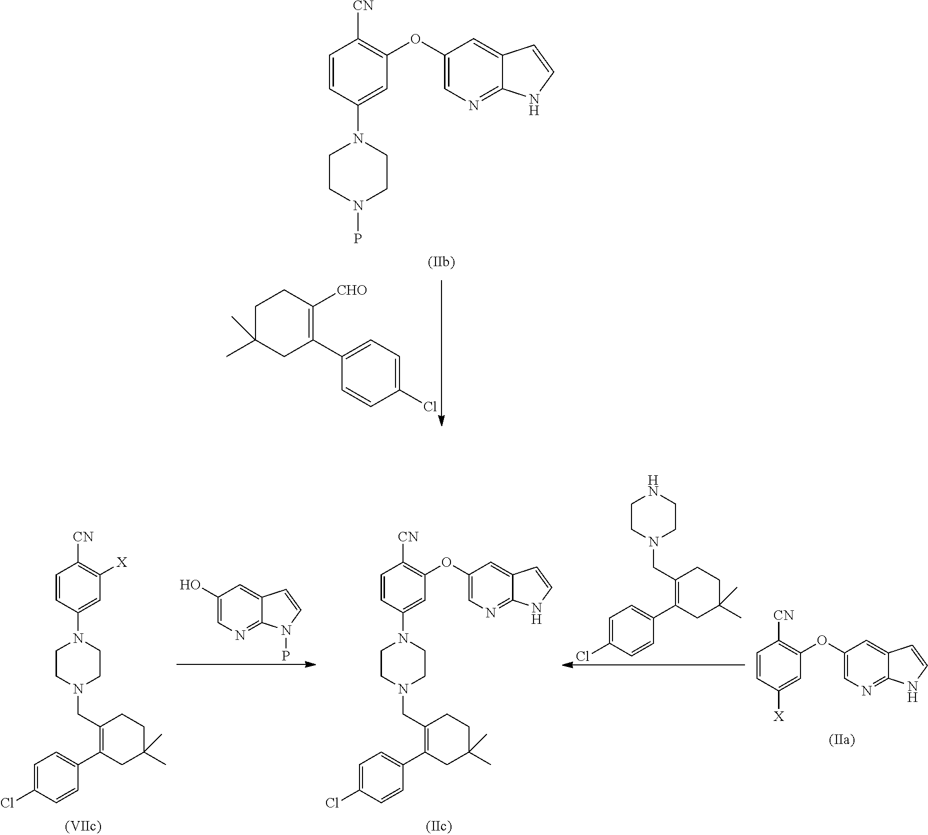

In an embodiment, step a), b) and c) of this aspect may be carried out by any method known in the art or according procedures described or exemplified in any aspect of the present application.

In an embodiment, steps a) and c) of this aspect may be carried out following similar methodology of steps a) and b) of the previous aspect.

Step b) of this aspect may be carried out by reacting a compound of formula (IIb) with 4′-chloro-5,5-dimethyl-3,4,5,6-tetrahydro-[1,1′-biphenyl]-2-carbaldehyde by any method known in the art or according procedures described or exemplified in any aspect of the present application.

In an embodiment, step b) may be carried out by reacting a compound of formula (IIb) with 4′-chloro-5,5-dimethyl-3,4,5,6-tetrahydro-[1,1′-biphenyl]-2-carbaldehyde in the presence of a suitable reducing agent and an inert solvent.

Suitable reducing agent that may be used in step b) may include, but not limited to sodium borohydride, sodium triacetoxy borohydride, sodium cyano borohydride-methanol, borane-pyridine or the like. In an alternate embodiment step b) may be carried out by catalytic hydrogenation method.

In an embodiment, unprotected cyano compound of formula (IIc) (wherein P is Hydrogen) may be obtained by deprotection the compound (IIc) (wherein P is a nitrogen protecting group).

In embodiments of this aspect, process of this aspect may be carried out at a suitable temperature of 0° C. to reflux temperature of the solvent used. The reaction may be carried out for sufficient time till the completion of hydrolysis for at least 1 hour or more.

In another aspect, the present application provides intermediate compounds of formula (IIa), formula (IIb) formula (IIc) useful to produce Venetoclax and intermediate compounds of formula (VIIb) and (VIIc) useful to produce compounds of formula (IIb) and (IIc), wherein X is any halogen such as fluorine, chlorine, Bromine or Iodine and P is hydrogen or any nitrogen protecting group such as BoC or Cbz.

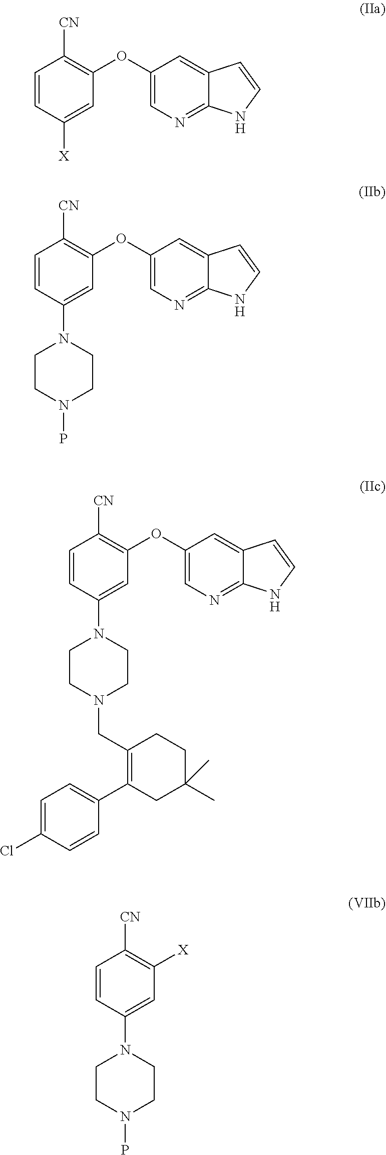

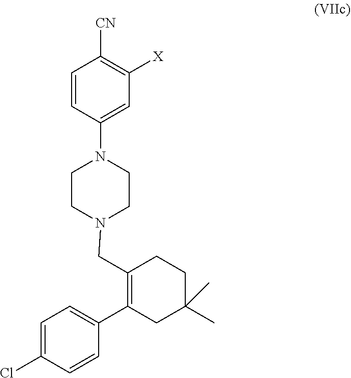

In another aspect, the present application provides compounds of formula (Xa), formula (Xb) and formula (Xc), wherein X is any halogen such as fluorine, chlorine, Bromine or Iodine and P is hydrogen or any nitrogen protecting group such as BoC or Cbz.

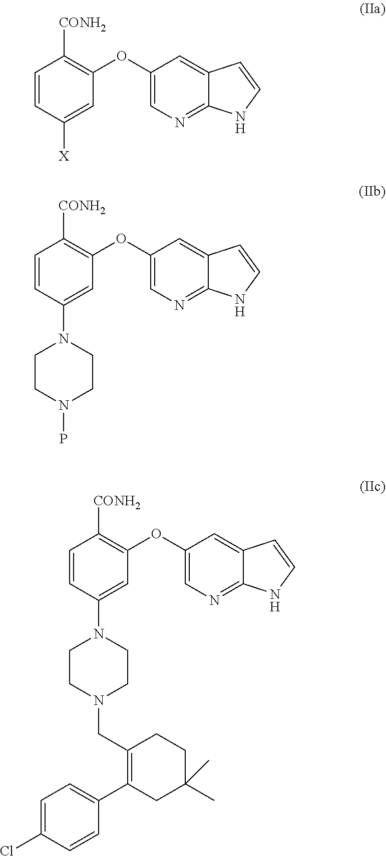

In another aspect, the present application provides a process for the preparation of Venetoclax, comprising the step of converting the compound of formula (IIIb) to Venetoclax;

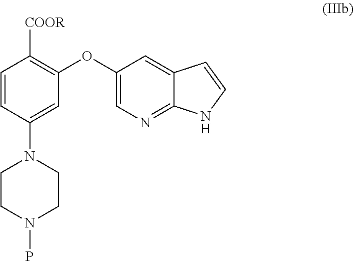

wherein P is hydrogen or any nitrogen protecting group such as BoC or Cbz and R may be selected from the group comprising of hydrogen, alkyl, aryl, arylalkyl or heteroaryl.

Compound of formula (IIIb) may be prepared according any suitable method known in the art or according to the procedures described or exemplified in any aspect of the present application.

In an embodiment, the compound of formula (IIIb) may be converted to Venetoclax by the process comprising the steps of reacting it with 3-nitro-4-(((tetrahydro-2H-pyran-4-yl)methyl)amino) benzene sulfonamide and 4′-chloro-5,5-dimethyl-3,4,5,6-tetrahydro-[1,1′-biphenyl]-2-carbaldehyde in either of the sequence as depicted below, according to any of the methods known in the art or procedures described or exemplified in any aspect of the present application.

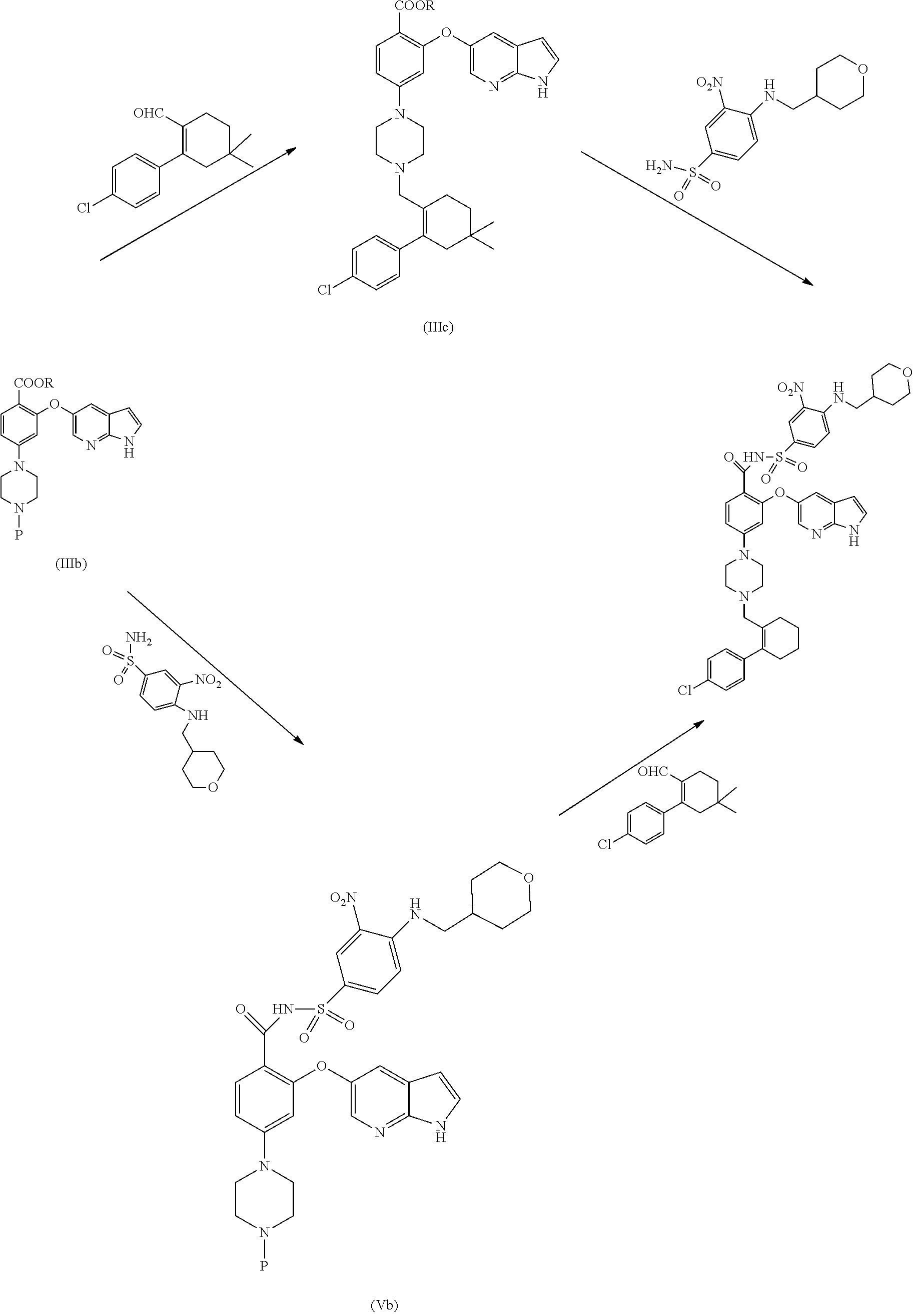

In another aspect, the present application provides a process for the preparation of Venetoclax, comprising the step of
a) reacting compound of formula (Ma) with optionally protected piperazine (or) reacting compound of formula (VIb) with optionally protected 1H-pyrrolo[2,3-b]pyridin-5-ol or its reactive derivative to obtain compound of formula (IIIb).

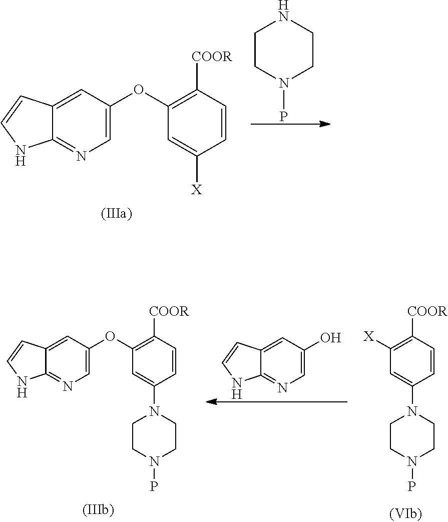

b) converting the compound of formula (IIIb) to Venetoclax.

In alternate embodiment, step a) of this aspect may be carried out by reacting a compound of formula (IIIa) with optionally protected piperazine by any method known in the art or according to procedure described or exemplified in any aspect of the present application.

In an embodiment, reaction of the compound of formula (IIIa) with optionally protected piperazine may be carried out in the presence of a suitable base, an inert solvent and optionally in the presence of a phase transfer catalyst.

In an embodiment, step a) of this aspect may be carried out by reacting 2-halo benzoate of formula (VIb) with optionally protected 1H-pyrrolo[2,3-b]pyridin-5-ol or its reactive derivative by any method known in the art or according to procedure described or exemplified in any aspect of the present application.

In an embodiment, 2-halo benzoate of formula (VIb) may be reacted with optionally protected 1H-pyrrolo[2,3-b]pyridin-5-ol or its reactive derivative in the presence of a suitable base, an inert solvent and optionally in the presence of a phase transfer catalyst. In an embodiment, reaction may be carried out in the presence of a solvent system comprising 1,4-dioxane.

Suitable base that may be used in this aspect may include, but not limited to hydroxides such as sodium hydroxide, potassium hydroxides; carbonates such as sodium carbonate, potassium carbonates; alkoxides such as sodium or potassium tert. butoxide; phosphates such as tribasic or dibasic potassium phosphate; or the like.

In embodiments of this aspect, unprotected compound of formula (IIIb) (wherein P is hydrogen) may be obtained by deprotection the compound (IIIb) (wherein P is a nitrogen protecting group) In embodiments of this aspect, process of this aspect may be carried out at a suitable temperature of 0° C. to reflux temperature of the solvent used. The reaction may be carried out for sufficient time till the completion of reaction for at least 1 hour or more.

Step b) of this aspect may be carried out by converting compound of formula (IIIb) to Venetoclax by any method known in the art or according the procedure described or exemplified in any aspect of the present application.

In an embodiment, compound of formula (IIIb) may be converted to Venetoclax according to any of the processes of previous aspect.

In another aspect, the present application provides a process for the preparation of Benzoate ester compound of formula (III) comprising the step of reacting 2-halo benzoate of formula (VI) with optionally protected 1H-pyrrolo[2,3-b]pyridin-5-ol or its reactive derivative

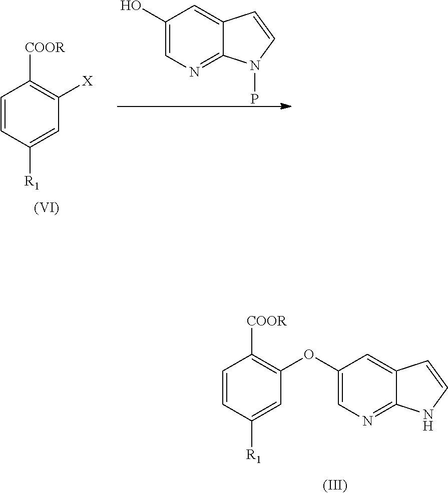

Wherein R may be selected from the group comprising of alkyl, aryl, arylalkyl or heteroaryl; R₁ may be selected from the group comprising of leaving group such as a halogen, optionally protected piperazine or a group of formula (A).

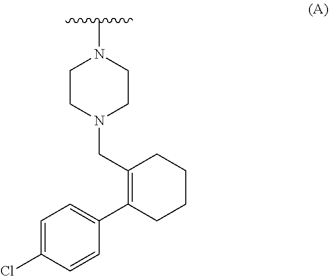 [SMILES: *N1CCN(CC2=C(c3ccc(Cl)cc3)CCCC2)CC1]

In an embodiment, a process for the preparation of benzoate ester compound of formula (IIIa), when X is a leaving group such as halogen, comprising the step of reacting 2,4-dihalo benzoate of formula (VIa) with optionally protected 1H-pyrrolo[2,3-b]pyridin-5-ol or its reactive derivative.

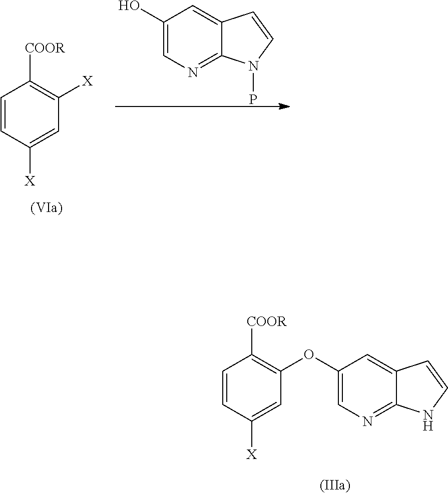

In an embodiment, a process for the preparation of benzoate ester compound of formula (IIIb), when P is a optionally protected piperazine, comprising the step of reacting 2-halo benzoate of formula (VIb) with optionally protected 1H-pyrrolo[2,3-b]pyridin-5-ol or its reactive derivative.

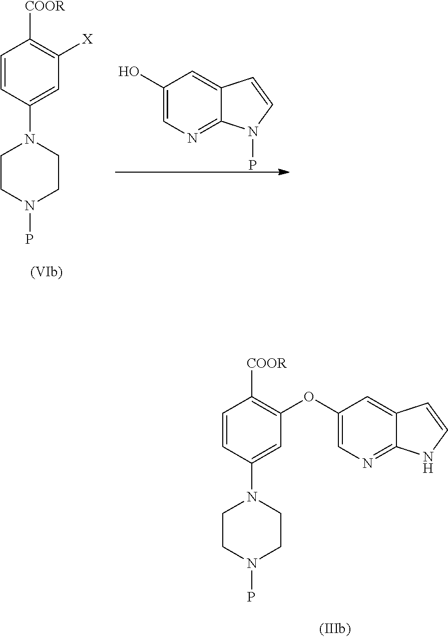

In an embodiment, a process for the preparation of benzoate ester compound of formula (IIIc), when R is a group of formula (A), comprising the step of reacting 2-halo benzoate of formula (VIc) with optionally protected 1H-pyrrolo[2,3-b]pyridin-5-ol or its reactive derivative.

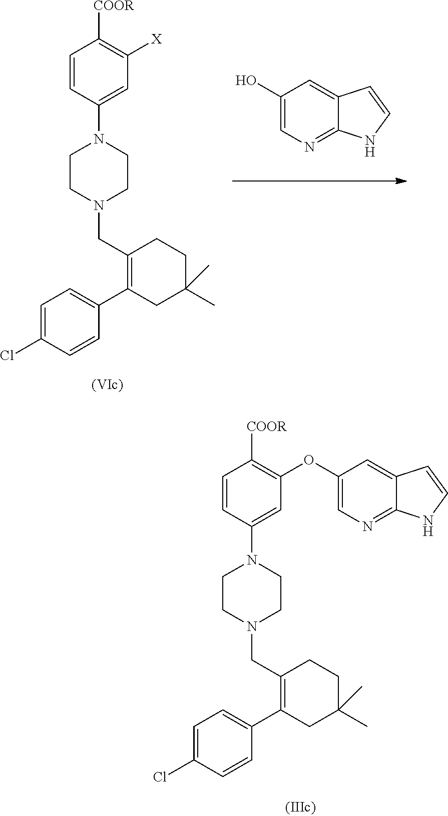

In an aspect, alternatively compound of formula (IIIc) may also be prepared from compound of formula (IIIa) and (IIIb) as depicted below.

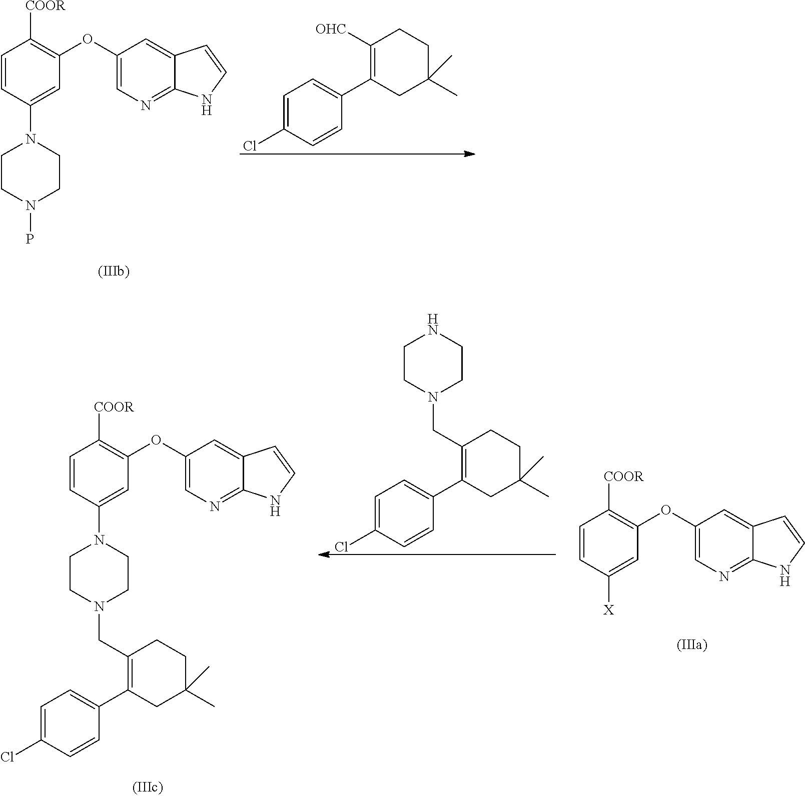

In another aspect, the present application provides a process for the preparation of compound of formula (IIa) or (IIIa), comprising the step of reacting a 2,4-dihalo benzene derivative of formula (VIIa) or (VIa), respectively with an optionally protected 1H-pyrrolo[2,3-b]pyridin-5-ol or its reactive derivative in the presence of suitable solvent system comprising 1,4-dioxane; wherein R₃ may be CN or COOR; R may be selected from the group comprising of alkyl, aryl, arylalkyl or heteroaryl and X is any halogen selected from the group comprising of fluorine, chlorine, Bromine or Iodine.

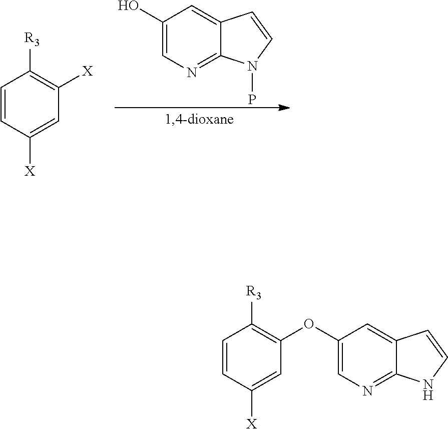

Inventors of the present application have identified that the solvent system comprising 1,4-dioxane results in the high selectivity of the desired position isomer i.e., ortho-isomer of the product compared to other known solvent systems for similar processes in the prior art such as diglyme as tabulated below.

A noteworthy observation by the inventors is that the undesired positional isomer i.e., para-isomer of the product can be controlled to minimum level during the reaction itself. It is very difficult to separate these positional isomers in the resultant mixture at this stage or in later stages of Venetoclax preparation. Further, it may require multiple purification steps to attain desired quality of the product. Hence, this solvent system avoids the time consuming and costly purification methods and yields the desired isomer at this stage itself.

| | | | | | | | Crude HPLC | |
|---|---|---|---|---|---|---|---|---|
| | | | | | | | analysis | |
| Reaction | | | Input | | Output | | Ortho | Para |
| condition | R₃ | Solvent | quantity | | quantity | | Product | product |
| K₃PO₄ 90° C., | CN | 1,4- | 200 | mg | 360 | mg | 93.64% | 0.67% |
| 16 h | | Dioxane | | | | | | |
| K₃PO₄, 115° C., | CN | Diglyme | 200 | mg | 250 | mg | 86.07% | 7.78% |
| 16 h | | | | | | | | |
| K₃PO₄, 90° C., 2 | COOMe | 1,4- | 1 | g | 1.30 | g | 74.23% | 0.14% |
| days | | Dioxane | | | | | | |
| K₃PO₄, 115° C., | COOMe | Diglyme | 1 | g | 1.03 | g | 75.32% | 2.34% |
| 2 days | | | | | | | | |

Starting materials used in any aspect of the instant application may be obtained from either commercially available sources or prepared according to the methods known in the art. Starting materials used in any aspect of the instant application may be purified according to the methods known in the art such as recrystallization, acid-base treatment, chromatography, fractional distillation, slurrying or the like, before using.

In an aspect, the present application provides crystalline form of Venetoclax, characterized by X-ray powder diffractogram pattern comprising the peaks at about 4.48, 11.63 and 16.19±0.2° 2θ.

In an alternate aspect the present application provides a crystalline form of Venetoclax characterized by the X-ray powder diffractogram of figure-28. In an embodiment, crystalline form of Venetoclax may obtained according to the process of example-44.

Venetoclax obtained according to any aspects of the instant patent application may be purified according to any of the methods known in the art recrystallization, acid-base treatment, chromatography or the like. Further, Venetoclax may be dried under suitable drying conditions such as air drying or vacuum drying.

In another aspect, the present application provides a process for the purification of Venetoclax through the formation of a suitable salt of Venetoclax or crystalline forms thereof followed by neutralization of the salt to Venetoclax according to any methods known in the art or procedures described or exemplified in any aspect of the instant application. Suitable salts may include but not limited to salts of an organic or inorganic acid such as acetic acid, formic acid, methanesulfonic acid, ethanesulfonic acid, maleic acid, malonic acid, fumaric acid, hydrogen halides like HCl, HBr, sulfuric acid, phosphoric acid or the like

In another aspect, the present application provides a pharmaceutical composition comprising Venetoclax or its crystalline form or its amorphous form obtained according any of the previous aspects and at least one additional pharmaceutically acceptable excipient.

In another aspect, the present application provides a pharmaceutical composition comprising crystalline forms of Venetoclax and at least one additional pharmaceutically acceptable excipient, wherein the crystalline form may be selected from the group comprising of RT1, RT2, RT3 Form RT4, RT5 or mixture thereof.

In another aspect, the present application provides Venetoclax, its crystalline or amorphous form or its acid addition salts according to instant application and pharmaceutical compositions thereof, wherein the chemical purity of Venetoclax or acid addition salt may be more than 99% by HPLC or more than 99.5% by HPLC or more than 99.9% by HPLC.

In another aspect, the present application provides Venetoclax, its crystalline form and pharmaceutical compositions thereof, wherein particle size (D90) of Venetoclax may be less than 100 microns or less than 50 microns or less than 20 microns.

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

### Definitions

The term “about” when used in the present application preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

The term “inert solvent” when used in the present application is a solvent that does not react with the reactants or reagent s under conditions that cause the chemical reaction indicated to take place.

The terms “amorphous form of Venetoclax” and “amorphous Venetoclax” indicate that the Venetoclax is present in substantially amorphous state in the composition (e.g. solid dispersion, adsorbate or pharmaceutical composition). “Substantially” amorphous denotes that 90%, preferably 95% or 99%, more preferably all of the Venetoclax being present in the solid dispersion, on the adsorbate or in the pharmaceutical composition is amorphous. In other words, an “amorphous” Venetoclax composition denotes a Venetoclax-containing composition, which does not contain substantial amounts, preferably does not contain noticeable amounts, of crystalline portions of Venetoclax e.g. measurable upon X-ray powder diffraction analysis.

The term “solid dispersion” when used in the present application, denotes a state where most of the Venetoclax, preferably 90%, 95% or all of the Venetoclax of the solid dispersion, is homogeneously molecularly dispersed in a solid polymer matrix. Preferably solid dispersion, relates to a molecular dispersion where the API (active pharmaceutical ingredient) and polymer molecules are uniformly but irregularly dispersed in a non-ordered way. In other words, in a solid dispersion, the two components (polymer and API) form a homogeneous one-phase system, where the particle size of the API in the solid dispersion is reduced to its molecular size. In a preferred embodiment, in the solid dispersion according to the present invention no chemical bonds can be detected between the API and the polymer. In order to arrive at such a solid dispersion, preferably solid solution, it is required to have a substantial amount of API dissolved in a suitable solvent at least at one time point during preparation of said solid dispersion.

The term “adsorbate” when used in the present application, specifies that the Venetoclax is, preferably evenly, and preferably homogeneously, distributed on the inner and/or outer surface of the particulate substrate.

An “alcohol” is an organic compound containing a carbon bound to a hydroxyl group. “C1-C6 alcohols” include, but are not limited to, methanol, ethanol, 2-nitroethanol,2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, phenol, glycerol, or the like.

An “aliphatic hydrocarbon” is a liquid hydrocarbon compound, which may be linear, branched, or cyclic and may be saturated or have as many as two double bonds. A liquid hydrocarbon compound that contains a six-carbon group having three double bonds in a ring is called“aromatic.” Examples of “C5-C8aliphatic or aromatic hydrocarbons” include, but are not limited to, n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, cyclohexane, methylcyclohexane, cycloheptane, benzene, toluene, ethylbenzene, m-xylene, o-xylene, p-xylene, trimethylbenzene, chlorobenzene, fluorobenzene, trifluorotoluene, anisole, or any mixtures thereof.

An “ester” is an organic compound containing a carboxyl group —(C═O)—O— bonded to two other carbon atoms. “C3-C6esters” include, but are not limited to, ethyl acetate, n-propyl acetate, n-butyl acetate, iso propyl acetate, isobutyl acetate, t-butyl acetate, ethyl formate, methyl acetate, methyl propanoate, ethyl propanoate, methyl butanoate, ethyl butanoate, or the like.

An “ether” is an organic compound containing an oxygen atom —O— bonded to two other carbon atoms. “C2-C6 ethers” include, but are not limited to, diethyl ether, diisopropyl ether, methyl t-butyl ether, glyme, diglyme, tetrahydrofuran, 2-methyltetrahydrofuran, 1,4-dioxane, dibutyl ether, dimethylfuran, 2-methoxyethanol, 2-ethoxyethanol, anisole, or the like.

A “halogenated hydrocarbon” is an organic compound containing a carbon bound to a halogen. Halogenated hydrocarbons include, but are not limited to, dichloromethane, 1,2-dichloroethane, trichloroethylene, perchloroethylene, 1,1,1-trichloroethane, 1,1,2-trichloroethane, chloroform, carbon tetrachloride, or the like.

A “ketone” is an organic compound containing a carbonyl group —(C═O)— bonded to two other carbon atoms. “C3-C6 ketones” include, but are not limited to, acetone, ethyl methyl ketone, diethyl ketone, methyl isobutyl ketone, ketones, or the like.

A “nitrile” is an organic compound containing a cyano —(C≡N) bonded to another carbon atom. “C2-C6Nitriles” include, but are not limited to, acetonitrile, propionitrile, butanenitrile, or the like.

### EXAMPLES

### Example-1: Preparation of Amorphous Form of Venetoclax

Venetoclax (0.5 g) was dissolved in dichloromethane (50 mL) at 45° C. and filtered the solution to make it particle free. The clear solution was taken into a Buchi flask and evaporated the solvent completely using rotavapour under vacuum at 50° C. to obtain the title compound. XRPD: Amorphous.

### Example-2: Preparation of Amorphous Form of Venetoclax

Venetoclax (0.75 g) was taken into a ball mill chamber and milled the compound for 2 hours with 400 RPM with 10 minutes time interval and recovered the title compound. XRPD: Amorphous.

### Example-3: Preparation of Amorphous Form of Venetoclax

Venetoclax (2 g) was dissolved in dichloromethane (180 mL) at 30° C. and filtered the solution to make it particle free. The clear solution was spray dried with 70% aspirator, flow rate of 6 mL/minute and inlet temperature of 60° C. and out let temperature of 40° C. to obtain the title compound. XRPD: Amorphous.

### Example-4: Preparation of Amorphous Solid Dispersion of Venetoclax with Syloid

Amorphous Venetoclax (0.18 g) and Syloid (0.18 g) were combined and ground in a mortar-pestle for 5 minutes at 30° C. to obtain title compound. XRPD: Amorphous

### Example-5: Preparation of Amorphous Solid Dispersion of Venetoclax and Povidone K-30

Venetoclax (0.5 g) and Povidone K-30 (0.5 g) were dissolved in dichloromethane (75 mL) at 30° C. and filtered the solution to make it particle free. The clear solution was taken into a Buchi flask and evaporated the solvent completely using rotavapour under vacuum at 45° C. to obtain title compound. XRPD: Amorphous.

### Example-6: Preparation of Amorphous Solid Dispersion of Venetoclax, Povidone K-30 and Syloid

Venetoclax (0.5 g) and Povidone K-30 (0.5 g) were dissolved in dichloromethane (75 mL) at 30° C. and filtered the solution to make it particle free. The above clear solution was taken into a Buchi flask and Syloid (0.5 g) was added. The solvent was evaporated completely form the mixture using rotavapour under vacuum at 45° C. to obtain title compound. XRPD: Amorphous.

### Example-7: Preparation of Amorphous Solid Dispersion of Venetoclax and HPMC-AS

Venetoclax (0.25 g) and HPMC-AS (0.25 g) were dissolved in dichloromethane (35 mL) at 30° C. and filtered the solution to make it particle free. The above clear solution was taken into a Buchi flask and the solvent was evaporated completely using rotavapour under vacuum at 45° C. to obtain title compound. XRPD: Amorphous.

### Example-8: Preparation of Amorphous Solid Dispersion of Venetoclax and Eudragit-RLPO

Venetoclax (0.25 g) and Eudragit-RLPO (0.25 g) were dissolved in dichloromethane (35 mL) at 30° C. and filtered the solution to make it particle free. The above clear solution was taken into a Buchi flask and the solvent was evaporated completely using rotavapour under vacuum at 45° C. to obtain title compound. XRPD: Amorphous.

### Example-9: Preparation of Amorphous Solid Dispersion of Venetoclax and HPC-L

Venetoclax (0.25 g) and HPC-L (0.25 g) were dissolved in dichloromethane (35 mL) at 30° C. and filtered the solution to make it particle free. The above clear solution was taken into a Buchi flask and the solvent was evaporated completely using rotavapour under vacuum at 45° C. to obtain title compound. XRPD: Amorphous.

### Example-10: Preparation of Amorphous Form of Venetoclax

Venetoclax (0.25 g) was dissolved in a mixture of dichloromethane (12 mL) and methanol (3 mL) at 30° C. and filtered the solution to make it particle free. The above clear solution was taken into a Buchi flask and the solvent was evaporated completely using rotavapour under vacuum at 60° C. to obtain title compound. XRPD: Amorphous.

### Example-11: Preparation of Amorphous Solid Dispersion of Venetoclax and Soluplus®

Venetoclax (0.25 g) and Soluplus® (0.25 g) were dissolved in dichloromethane (35 mL) at 30° C. and filtered the solution to make it particle free. The above clear solution was taken into a Buchi flask and the solvent was evaporated completely using rotavapour under vacuum at 45° C. to obtain title compound. XRPD: Amorphous.

### Example-12: Preparation of Trifluoroacetic Acid (TFA) Salt of Venetoclax

Venetoclax (500 mg) was dissolved in acetone (10 mL) at 28° C. and trifluoroacetic acid (TFA) (0.048 mL) was added at the same temperature. The reaction mixture was stirred for 4 hours at 28° C. and filtered the solid. The solid was washed with acetone (10 mL) and dried for 20 hours under reduced pressure at 28° C. and for 45 minutes at 50° C. to obtain the title compound with melting range of 230-232° C. Yield: 545 mg and HPLC purity: 99.37%

### Example-13: Preparation of Oxalic Acid Salt of Venetoclax

Venetoclax (500 mg) was dissolved in ethanol (10 mL) at 28° C. and oxalic acid (57.0 mg in 5 mL of ethanol) was added at the same temperature. The reaction mixture was heated to 90° C. and stirred at this temperature for 3 hours. Cooled the reaction mixture to 28° C. and stirred for 14 hours at the same temperature. The solid was filtered and washed with ethanol (10 mL). The solid was dried for 24 hours at under reduced pressure at 28° C. and at 50° C. for 45 minutes to obtain the title compound with melting range of 220-223° C. Yield: 369 mg and HPLC purity: 99.08%

### Example-14: Preparation of Maleic Acid Salt of Venetoclax

Venetoclax (500 mg) was dissolved in acetone (8 mL) at 28° C. and maleic acid (73.5 mg in 2 mL of acetone) was added at the same temperature. The reaction mixture was stirred for 17 hours at 28° C. and filtered the solid. The solid was washed with acetone (10 mL) and dried for 6 hours at 28° C. under reduced pressure and 45 minutes at 50° C. under reduced pressure to obtain the title compound with melting range of 204-206° C. Yield: 372 mg and HPLC purity: 99.518%

### Example-15: Preparation of Isethionic Acid Salt of Venetoclax

Venetoclax (500 mg) was dissolved in acetone (10 mL) at 28° C. and isethionic acid (0.049 mL) was added at the same temperature. The reaction mixture was stirred for 17 hours at 28° C. and filtered the solid. The solid was washed with acetone (10 mL) and dried for 6 hours at 28° C. under reduced pressure and 45 minutes at 50° C. under reduced pressure to obtain the title compound with melting range of 172-174° C. Yield: 500 mg and HPLC purity: 99.539%

### Example-16: Preparation of Hydrochloride Salt of Venetoclax

Venetoclax (500 mg) was dissolved in acetone (10 mL) at 28° C. and hydrochloric acid (0.25 mL of 4 M hydrochloride in 1,4-dioxane) was added at the same temperature. The reaction mixture was stirred for 17 hours at 28° C. and filtered the solid. The solid was washed with acetone (10 mL) and dried for 6 hours at 28° C. under reduced pressure and 45 minutes at 50° C. under reduced pressure to obtain the title compound with melting range of 200-202° C. Yield: 440 mg and HPLC purity: 99.278%

### Example-17: Preparation of Ortho-Phosphoric Acid Salt of Venetoclax

Venetoclax (500 mg) was dissolved in acetone (10 mL) at 28° C. and ortho-phosphoric acid (0.032 mL) was added at the same temperature. The reaction mixture was stirred for 16.5 hours at 28° C. and filtered the solid. The solid was washed with acetone (10 mL) and dried for 6 hours at 28° C. under reduced pressure and 45 minutes at 50° C. under reduced pressure to obtain the title compound with melting range of 200-202° C. Yield: 400 mg and HPLC purity: 99.165%

### Example-18: Preparation of Citric Acid Salt of Venetoclax

Venetoclax (500 mg) was dissolved in acetone (8 mL) at 28° C. and citric acid monohydrate (133 mg in 2 mL of acetone) was added at the same temperature. The reaction mixture was stirred for 18 hours at 28° C. and filtered the solid. The solid was washed with acetone (10 mL) and dried for 6 hours at 28° C. under reduced pressure and 45 minutes at 50° C. under reduced pressure to obtain the title compound with melting range of 168-170° C. Yield: 532 mg and HPLC purity: 98.796%

### Example-19: Preparation of Methanesulfonic Acid Salt of Venetoclax

Venetoclax (500 mg) was dissolved in acetone (10 mL) at 28° C. and methanesulfonic acid (0.04 mL) was added at the same temperature. The reaction mixture was stirred for 17 hours at 28° C. and filtered the solid. The solid was washed with acetone (10 mL) and dried for 12 hours at 28° C. under reduced pressure and 45 minutes at 50° C. under reduced pressure to obtain the title compound with melting range of 165-168° C. Yield: 448 mg and HPLC purity: 99.51%

### Example-20: Preparation of Acetic Acid Salt of Venetoclax

Acetic acid (0.036 mL) was added to a mixture of Venetoclax (500 mg) in acetone (10 mL) at 28° C. and heated to 60° C. The reaction mixture was stirred for 1 hour at 60° C. and cooled to 28° C. The reaction mixture was stirred for 14 hours at the same temperature and filtered. The solid was washed with acetone (10 mL) and dried for 6 hours at 28° C. under reduced pressure and 45 minutes at 50° C. under reduced pressure to obtain the title compound with melting range of 158-160° C. Yield: 452 mg and HPLC purity: 99.333%

### Example-21: Preparation of Crystalline Form RT1 of Venetoclax

Venetoclax (1 g) was dissolved in benzyl alcohol (2 mL) at 90° C. and cooled the solution to 50° C. n-heptane (18 mL) was added to the solution at 50° C. and cooled further to 25° C. Methyl tert. Butyl ether (10 mL) was added to the reaction mixture and stirred for 5 minutes. The solid was filtered under vacuum for about 10 minutes and dried at 50° C. for one hour in air tray drier to obtain the title compound.

### Example-22: Preparation of Crystalline Form RT2 of Venetoclax

A mixture of Venetoclax (1 g) in methyl isopropyl ketone (20 mL) was heated to 45° C. and stirred at the same temperature for about 8 hours. Cooled the reaction mixture to 25° C. and filtered under vacuum for about 5 minutes. The solid was dried at 45° C. for one hour in air tray drier to obtain the title compound.

### Example-23: Preparation of Crystalline Form RT3 of Venetoclax

Venetoclax (20 g) was dissolved in methylene chloride (1 L) at 28° C. and water (400 mL) was added to this solution. The mixture was stirred for 30 minutes at 28° C. and separated the organic layer. The aqueous layer was extracted with methylene chloride (300 mL). The combined organic layer was washed with water (400 mL) and dried over anhydrous sodium sulfate. The solvent was evaporated from the organic layer under reduced pressure at 50° C. and the solid was dried under reduced pressure for 6 hours at 50° C. to obtain the title compound with melting range of 141-146° C. Yield: 19.11 g and HPLC purity: 99.290%

### Example-24: Preparation of Amorphous Form of Venetoclax

Venetoclax (20 g) was dissolved in DMSO (60 mL) at 90° C. and filtered the solution under hot condition. The hot filtrate was added to water (600 mL) at 25° C. and stirred for about 10 minutes at the same temperature. The solid was filtered and dried under reduced pressure initially followed by drying in air tray drier at 45° C. for 7 hours to obtain the title compound. Yield: 18.5 g

### Example-25: Preparation of Crystalline Form RT4 of Venetoclax

Venetoclax (1 g) was suspended in methyl isobutyl ketone (15 mL) and heated to 45° C. The mixture was stirred at the same temperature for about 21 hours. Cooled the reaction mixture to 25° C. and filtered the solid under vacuum. The solid was dried at 50° C. for 7 hours in air tray drier to obtain the title compound.

### Example-26: Preparation of Crystalline Form RT5 of Venetoclax

Venetoclax (20 g) was suspended in 1,4-dioxane (7 mL) at 25° C. and the mixture was stirred for 30 minutes at 25° C. The solid was filtered and dried in air tray drier at 90° C. for 1 hour. The solid was suspended in water (30 mL) at 50° C. for 2 hours and the solid was filtered. The solid was dried in air tray drier at 90° C. for 3 hours to obtain the title compound.

### Example-27: Preparation of Crystalline Form RT2 of Venetoclax

Venetoclax (750 mg) was suspended in methyl tert. Butyl ether (15 mL) at 25° C. and stirred for 24 hours at the same temperature. The solid was filtered under vacuum and dried at 45° C. for 10 hours in air tray drier to obtain the title compound.

### Example-28: Preparation of Methyl 2-((1H-pyrrolo[2,3-b]pyridin-5-yl)oxy)-4-fluorobenzoate

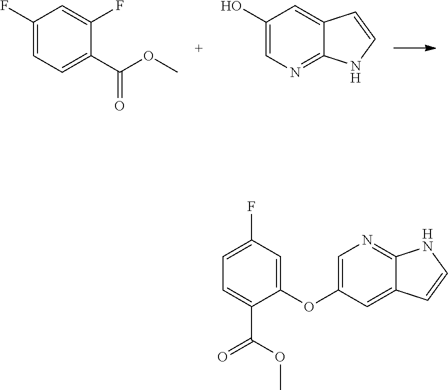

To a mixture of methyl 2,4-difluoro benzoate (1.0 g) in 1,4-dioxane (20 mL), 5-hydroxy pyrrolo[2,3-b]pyridine (779 mg) and K₃PO₄ (1.47 g) were added at 34° C. and heated to 90° C. The reaction mixture is stirred at the same temperature for 23 hours. K₃PO₄ (396 mg) was added at 90° C. to the reaction mixture and stirred for another 24 hours at the same temperature. Cooled the reaction mixture to 32° C. and filtered on a celite bed. Washed the celite bed with ethyl acetate (20 mL) and evaporated the solvent in the filtrate to obtain crude product. The crude product was purified by column chromatography using 60-120 silica gel mesh and 10-50% ethyl acetate-hexane as eluent to obtain the title compound as white solid. Yield: 588 mg; Purity by HPLC: 98.73%

### Example-29: Preparation of 2-((1H-pyrrolo[2,3-b]pyridin-5-yl)oxy)-4-fluorobenzoic Acid

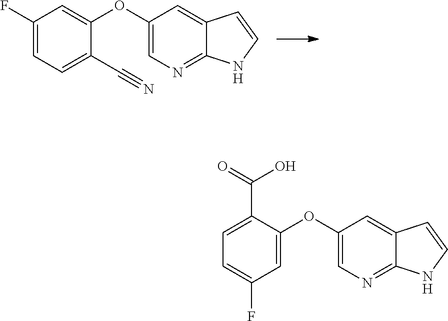

A mixture of 2-((1H-pyrrolo[2,3-b]pyridin-5-yl)oxy)-4-fluorobenzonitrile (100 mg) and concentrated hydrochloric acid (2 mL) was heated to 90° C. and stirred for 19 hours at the same temperature. The reaction mixture was cooled to 32° C. and removed hydrochloric acid by co-distilling with toluene (2×10 mL). The product was washed with methyl tert. Butyl ether (2×5 mL) and dried under vacuum at 50° C. for 15 minutes to obtain the title compound as brown solid. Yield: 102 mg; Purity by HPLC: 86%

### Example-30: Preparation of 2-((1H-pyrrolo[2,3-b]pyridin-5-yl)oxy)-4-fluoro benzonitrile

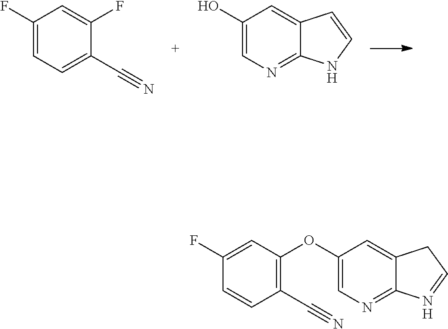

To a mixture of 2,4 difluoro benzonitrile (200 mg) in 1,4-dioxane (5 mL), 5-hydroxy pyrrolo[2,3-b]pyridine (192 mg) and K₃PO₄ (457 mg) were added at 32° C. and heated to 90° C. The reaction mixture is stirred at the same temperature for 20 hours. Filtered the reaction mixture on celite bed and evaporated the solvent in the filtrate to obtain crude product. The crude product was purified by column chromatography using 60-120 silica gel mesh and 10-50% ethyl acetate-hexane as eluent to obtain the title compound as white solid. Yield: 270 mg; Purity by HPLC: 98.86%

### Example-31: Preparation of tert-butyl 4-(4-cyano-3-fluorophenyl)piperazine-1-carboxylate

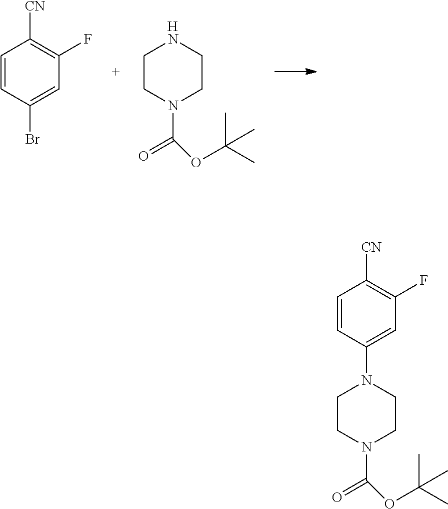

4-bromo-2-fluorobenzonitrile (0.5 g), tert-butyl piperazine-1-carboxylate (0.488 g), cesium carbonate (2.44 g), Tri(o-tolyl)phosphine (P(o-tol)3) (0.6 g) were taken in Toluene (5 mL) in a seal tube at 29° C. and purged with argon gas for 60 minutes. Pd(OAc)₂ (0.22 g) was added to this reaction mixture at 29° C. and again purged with argon gas for 30 minutes at the same temperature. The reaction mixture was heated to 90° C. for 48 hours and then diluted with water (20 mL). Reaction mixture was extracted with ethyl acetate (2×20 mL) and the combined organic layer was dried over sodium sulfate and evaporated to obtain crude product, which was purified by column chromatography using 60-120 silica mesh and 5-20% ethyl acetate-Hexane as eluent to obtain title compound as brown solid. Yield: 230 mg

### Example-32: Preparation of tert-butyl 4-(3-(1H-pyrrolo[2,3-b]pyridin-5-yloxy)-4-cyanophenyl)piperazine-1-carboxylate

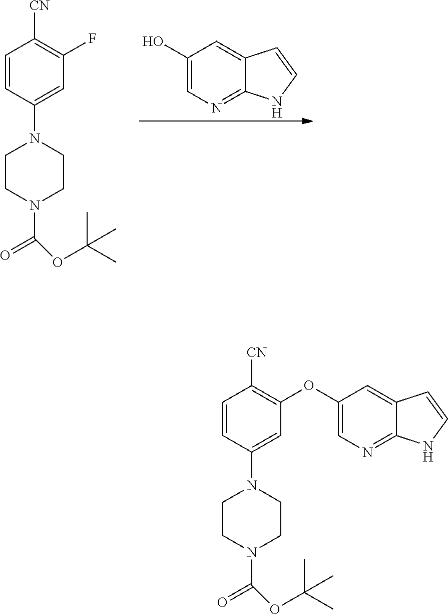 [SMILES: CC(C)(C)OC(=O)N1CCN(c2ccc(C#N)c(F)c2)CC1 ; Oc1cnc2[nH]ccc2c1 ; CC(C)(C)OC(=O)N1CCN(c2ccc(C#N)c(Oc3cnc4[nH]ccc4c3)c2)CC1]

Tert-butyl 4-(4-cyano-3-fluorophenyl)piperazine-1-carboxylate (150 mg), 5-hydroxy aza indole (0.065 g) and K₃PO₄ (0.2308 g) were taken in 1,4-dioxane (3 mL). The reaction mixture was heated to 90° C. for 24 hours. The solvent was evaporated completely to obtain the title compound.

### Example-33: Preparation of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzonitrile

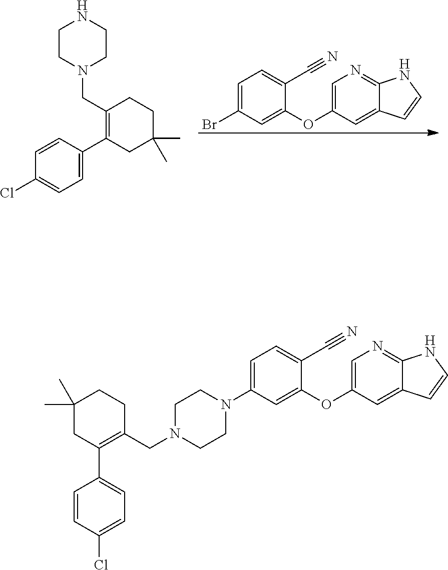

Combined 2-((1H-pyrrolo[2,3-b]pyridin-5-yl)oxy)-4-bromobenzonitrile (6.6 g) and 1-((4′-chloro-5,5-dimethyl-3,4,5,6-tetrahydro-[1,1′-biphenyl]-2-yl)methyl)piperazine (7.7 g) in tetrahydrofuran (200 mL). To the reaction mixture sodium tert. Butoxide (6.0 g) and (4-(N,N-Dimethylamino)phenyl)di-tert-butyl phosphine (A-phos) (445 mg) were added at 30° C. and degassed with Argon for 30 minutes under stirring at the same temperature. Tris(dibenzylideneacetone)dipalladium(0)-chloroform adduct (870 mg) was added and degassed for 5 minutes at 30° C. The reaction mixture was heated to 70° C. and stirred for 15 hours at the same temperature. The reaction mixture was cooled and filtered on celite bed. The celite bed was washed with tetrahydrofuran (2×50 mL) and evaporated the solvent in the filtrate. The crude product was dissolved in the ethyl acetate (100 mL) and washed with water (100 mL), saturated sodium bicarbonate solution (70 mL), 10% solution of L-cysteine (100 mL) and brine solution (60 mL). The organic solution was dried over sodium sulfate and evaporated the solvent under reduced pressure. The crude product was purified by column chromatography using 100-200 mesh silica gel and 40-50% ethyl acetate-hexane as eluent to obtain the title compound as yellow solid. Yield: 9.5 g; Purity by HPLC: 98.30%

### Example-34: Preparation of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzonitrile

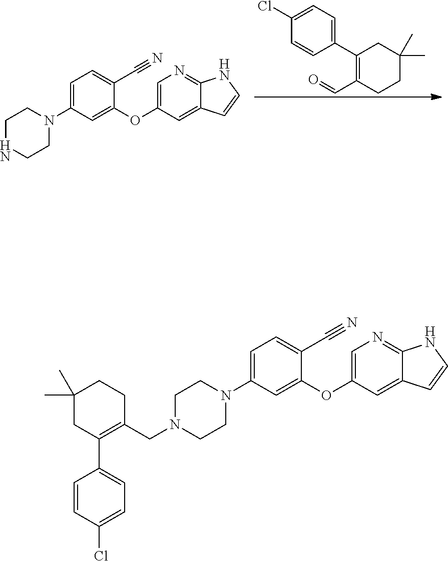 [SMILES: N#Cc1ccc(N2CCNCC2)cc1Oc1cnc2[nH]ccc2c1 ; CC1(C)CCC(C=O)=C(c2ccc(Cl)cc2)C1 ; CC1(C)CCC(CN2CCN(c3ccc(C#N)c(Oc4cnc5[nH]ccc5c4)c3)CC2)=C(c2ccc(Cl)cc2)C1]

4′-chloro-5,5-dimethyl-3,4,5,6-tetrahydro-[1,1′-biphenyl]-2-carbaldehyde (0.025 g) was dissolved in tetrahydrofuran (2 mL) and the reaction mixture was cooled to 5° C. 2-((1H-pyrrolo[2,3-b]pyridin-5-yl)oxy)-4-(piperazin-1-yl)benzonitrile (35 mg) and sodium triacetoxyborohydride (27.6 mg) were added to the reaction mixture. The reaction mixture was allowed to attain 30° C. and stirred for 24 hours at the same temperature. The reaction mixture was quenched with saturated aqueous ammonium chloride solution (2 mL) and extracted with ethyl acetate (2×5 mL) and washed with brine solution (2×5 mL). The separated organic layer was dried over sodium sulfate and evaporated the solvent completely. The crude compound was purified by column chromatography using 60-120 silica gel mesh and 20% ethyl acetate-hexane as eluent to obtain the title compound as an off-white solid. Yield: 7.5 mg.

### Example-35: Preparation of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid and 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzamide

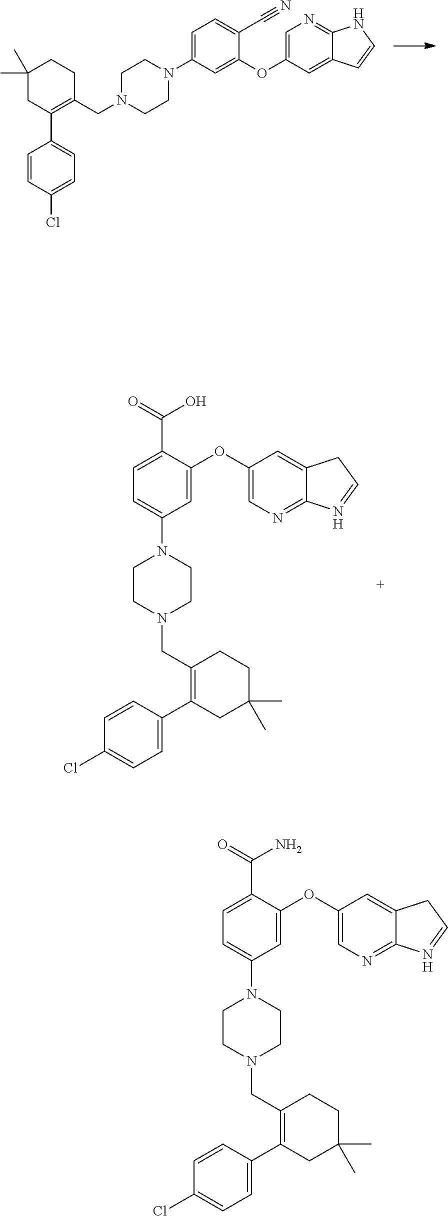

A mixture of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzonitrile (0.5 g), potassium hydroxide (0.5 g), ethanol (5 mL) and water (5 mL) was heated and stirred at 60° C. for 24 hours and 140° C. for 24 hours to obtain the mixture of title compounds. Individual compounds of benzamide and benzoic acid were separated using chromatography.

### Example-36: Preparation of 2-((1H-pyrrolo[2,3-b]pyridin-5-yl)oxy)-4-bromo benzonitrile

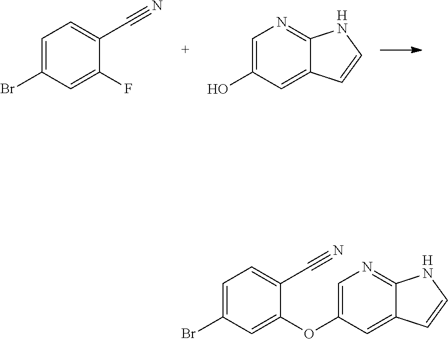

A mixture of 2-fluoro 4-bromo benzonitrile (5 g) and 5-hydroxy pyrrolo[2,3-b]pyridine (3.35 g) in dimethylformamide (20 mL) was cooled to 2° C. under nitrogen atmosphere. Sodium tert.butoxide (2.52 g) in dimethylformamide (10 mL) was added to reaction mixture in 15 minutes and allowed to attain 30° C. The reaction mixture is stirred at the same temperature for 2 hours. Cooled the reaction mixture to 5° C. and 0.1 equivalent of sodium tert.butoxide was added. Allowed the reaction mixture to attain 30° C. and stirred at the same temperature for 2 hours. Quenched the reaction mixture with water (200 mL) and stirred for 30 minutes. Filtered the reaction mixture and washed with water (50 mL) and n-hexane (50 mL) and evaporated the solvent under reduced pressure to obtain crude product. The crude product suspended in ethyl acetate (76 mL) and heated to reflux and evaporated the solvent completely. The product was stirred with 20% ethyl acetate/hexane (76 mL) for 30 minutes and filtered the solid and dried under vacuum to obtain the title compound as light brown solid. Yield: 6.7 g; Purity by HPLC: 98.44%

### Example-37: Preparation of tert-butyl 4-(3-((1H-pyrrolo[2,3-b]pyridin-5-yl)oxy)-4-cyanophenyl)piperazine-1-carboxylate

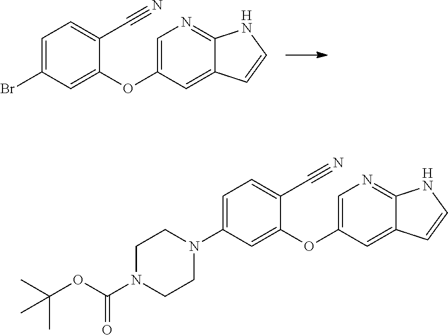

A mixture of 2-((1H-pyrrolo[2,3-b]pyridin-5-yl)oxy)-4-bromobenzonitrile (0.5 g), tert-butyl piperazine-1-carboxylate (0.34 g) and sodium tert.butoxide (0.45 g) in tetrahydrofuran (15 mL) was degassed with argon gas for 20 minutes at 31° C. (4-(N,N-Dimethylamino)phenyl)di-tert-butyl phosphine (A-phos) (33.7 mg) and Tris(dibenzylideneacetone)dipalladium(0)-chloroform adduct (65.9 mg) were added to the reaction mixture and heated to 60° C. The reaction mixture was stirred at the same temperature for 24 hours and cooled to 30° C. (4-(N,N-Dimethylamino)phenyl)di-tert-butyl phosphine (A-phos) (16 mg) and Tris(dibenzylideneacetone)dipalladium(0)-chloroform adduct (32 mg) were added to the reaction mixture and heated to 60° C. The reaction mixture was stirred at the same temperature for 24 hours and cooled to 30° C. Reaction mixture was filtered on celite bed and washed with ethyl acetate (20 mL). Evaporated the solvent in the filtrate and the crude product was purified by column chromatography using 40% ethyl acetate-hexane as eluent to obtain the title compound as off-white solid. Yield: 0.25 g; Purity by HPLC: 91.07%

### Example-38: Preparation of 2-((1H-pyrrolo[2,3-b]pyridin-5-yl)oxy)-4-(4-(tert-butoxycarbonyl)piperazin-1-yl)benzoic acid and 2-((1H-pyrrolo[2,3-b]pyridin-5-yl)oxy)-4-(4-(tert-butoxycarbonyl)piperazin-1-yl)benzamide

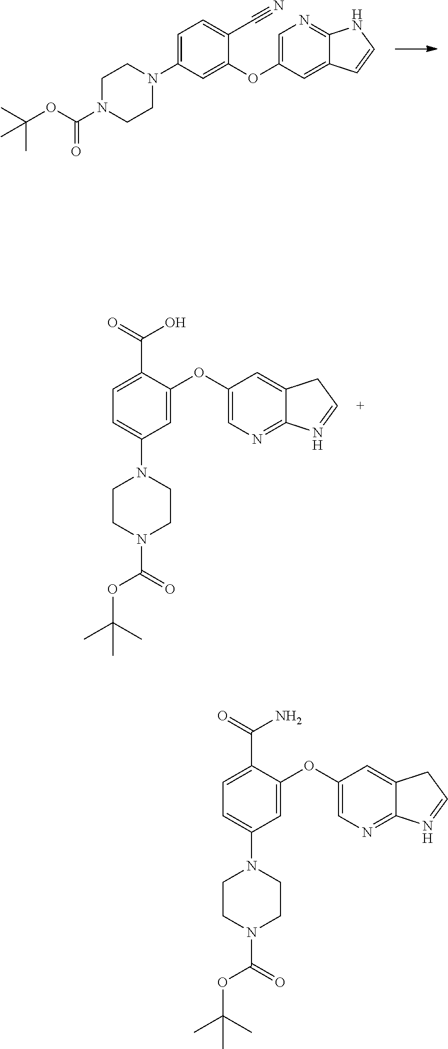

Tert-butyl4-(3-((1H-pyrrolo[2,3-b]pyridin-5-yl)oxy)-4-cyanophenyl)piperazine-1-carboxylate (40 mg) was dissolved in a mixture of ethanol (2 mL) and water (2 mL) at 31° C. Potassium hydroxide (53.5 mg) was added and heated the reaction mixture. The reaction mixture was stirred at 90° C. for 24 hours and cooled to 32° C. The solvent was evaporated completely under reduced pressure and the pH of the reaction mixture was adjusted to 5. The reaction mixture was extracted with ethyl acetate (2×5 mL) and the separated organic layer was washed with brine solution (5 mL). The organic layer was dried over sodium sulfate and evaporated the solvent completely mixture of title compounds. Individual compounds of benzamide and benzoic acid were separated by chromatography to obtain the title compounds.

### Example-39: Preparation of tert-butyl 4-(3-((1H-pyrrolo[2,3-b]pyridin-5-yl)oxy)-4-(methoxycarbonyl)phenyl)piperazine-1-carboxylate

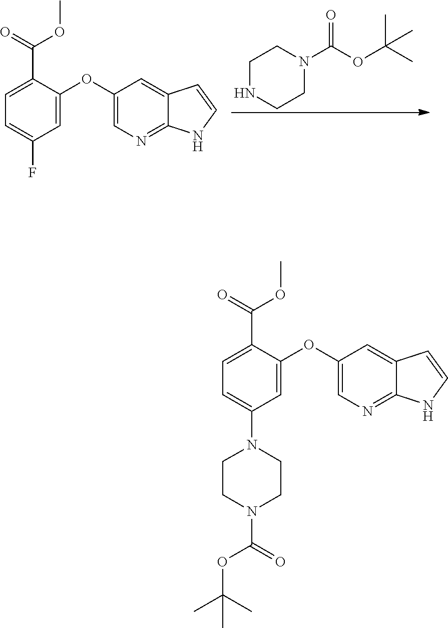

Methyl 2-((1H-pyrrolo[2,3-b]pyridin-5-yl)oxy)-4-fluorobenzoate (300 mg) was dissolved in dimethyl sulfoxide (6 mL). Tert-butyl piperazine-1-carboxylate (273 mg) and dibasic potassium phosphate (730 mg) were added to the reaction mixture and heated to 120° C. The reaction mixture was stirred at the same temperature for 30 hours and cooled to 32° C. Quenched the reaction mixture with cold water (10 mL) in 10 minutes and filtered the solid obtained. Washed the product with water (10 mL) and dissolved in ethyl acetate (10 mL). The organic solution was washed with water (2×10 mL) and brine solution (10 mL). Separated the organic layer and dried over sodium sulfate. The crude product was purified by column chromatography using 60-120 silica gel mesh and 50% ethyl acetate-hexane as eluent to obtain the title compound as white solid. Yield: 210 mg; Purity by HPLC: 96.51%

### Example-40: Preparation of methyl 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoate

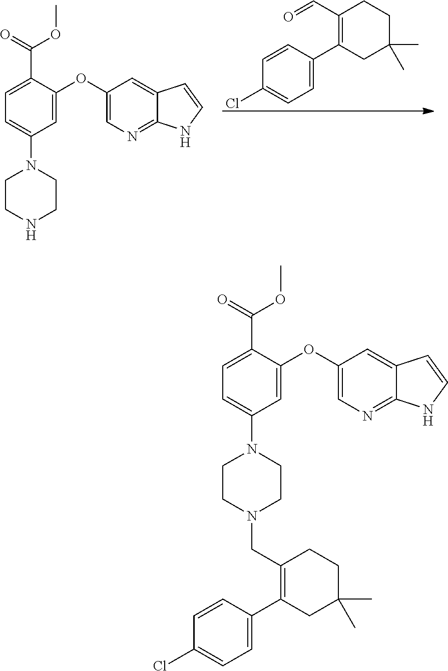

Methyl 2-((1H-pyrrolo[2,3-b]pyridin-5-yl)oxy)-4-(piperazin-1-yl)benzoate (0.1 g) was dissolved in tetrahydrofuran (3 mL) and 4′-chloro-5,5-dimethyl-3,4,5,6-tetrahydro-[1,1′-biphenyl]-2-carbaldehyde (0.077 g) was added at 32° C. and stirred for 10 minutes at the same temperature. The reaction mixture was cooled to 5° C. and sodium triacetoxy borohydride (0.09 g) was added. The reaction mixture was stirred at 5° C. for 20 hours and quenched with saturated aqueous ammonium chloride solution (5 mL). The reaction mixture was extracted with ethyl acetate (2×10 mL) and washed with brine solution (10 mL). The separated organic layer was dried over sodium sulfate and evaporated the solvent completely. The crude compound was purified by column chromatography using 60-120 silica gel mesh and 30% ethyl acetate-hexane as eluent to obtain the title compound as an off-white solid. Yield: 48.2 mg; Purity by HPLC: 97.5%

### Example-41: Preparation of methyl 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoate

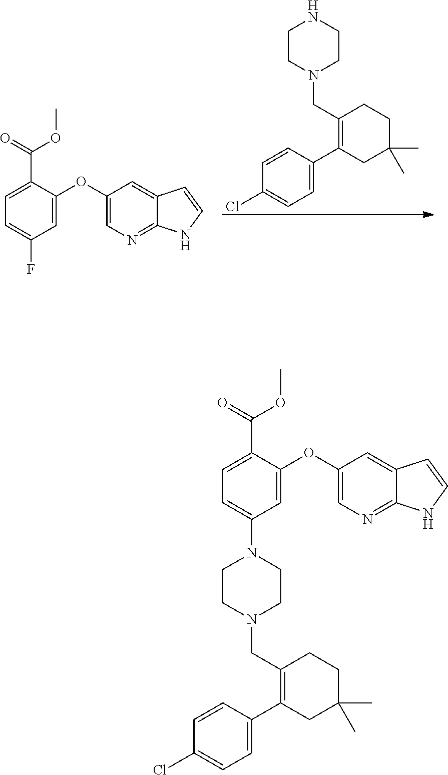

1-((4′-chloro-5,5-dimethyl-3,4,5,6-tetrahydro-[1,1′-biphenyl]-2-yl)methyl)piperazine (12.3 g) was dissolved in dimethyl sulfoxide (85 mL) under nitrogen atmosphere and added dibasic potassium phosphate (20.6 g) at 29° C. and stirred for 5 minutes at the same temperature. Methyl 2-((1H-pyrrolo[2,3-b]pyridin-5-yl)oxy)-4-fluorobenzoate (8.5 g) was added to the reaction mixture at 29° C. and stirred for 5 minutes at the same temperature. Heated the reaction mixture to 120° C. and stirred for 24 hours at the same temperature. The reaction mixture was cooled to 30° C. and quenched with cold water (255 mL) slowly in 20 minutes. The reaction mixture was stirred for 30 minutes at the 30° C. and filtered. The compound was dissolved in ethyl acetate (85 mL) and washed with saturated aqueous sodium bicarbonate (2×50 mL) and brines solution (25 mL). The separated organic layer was dried over sodium sulfate and evaporated the solvent completely to obtain crude compound. The crude compound was purified by column chromatography using 60-120 silica gel mesh and 60% ethyl acetate-hexane as eluent followed by recrystallization in diethyl ether and petroleum ether to obtain title compound as white solid. Yield: 11.4 g; Purity by HPLC: 99.052%

### Example-42: Preparation of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic Acid

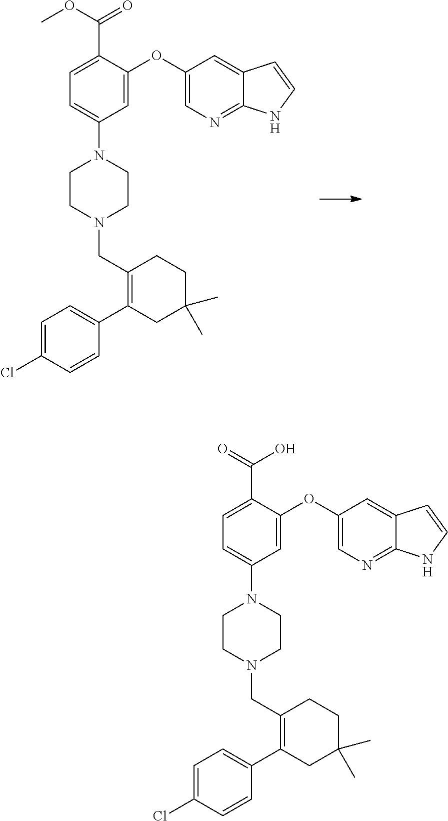

To a mixture of methyl 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoate (11.5 g) in tetrahydrofuran (115 mL), a solution of lithium hydroxide in water (8.2 g in 57 mL) was added at 32° C. The reaction mixture was heated and stirred at 65° C. for 44 hours and at 40° C. for 20 hours. The solvent was evaporated under reduced pressure at 50° C. and distilled the reaction mixture azeotropically with toluene (2×50 mL). Reaction mixture was washed with ethyl acetate (2×200 mL) and neutralized with 10% sodium dihydrogen phosphate solution (200 mL). Reaction mixture was extracted the ethyl acetate (3×150 mL) and the combined organic layer was washed with water (2×100 mL), brine solution (100 mL). The organic layer was dried over sodium sulfate and evaporated the solvent under reduced pressure to obtain the crude product as yellow solid. The crude product was recrystallized from mixture of 2-methyl tetrahydrofuran and heptane to obtain the title compound. Yield: 10.2 g; Purity by HPCL: 99.28%

### Example-43: Preparation of Venetoclax

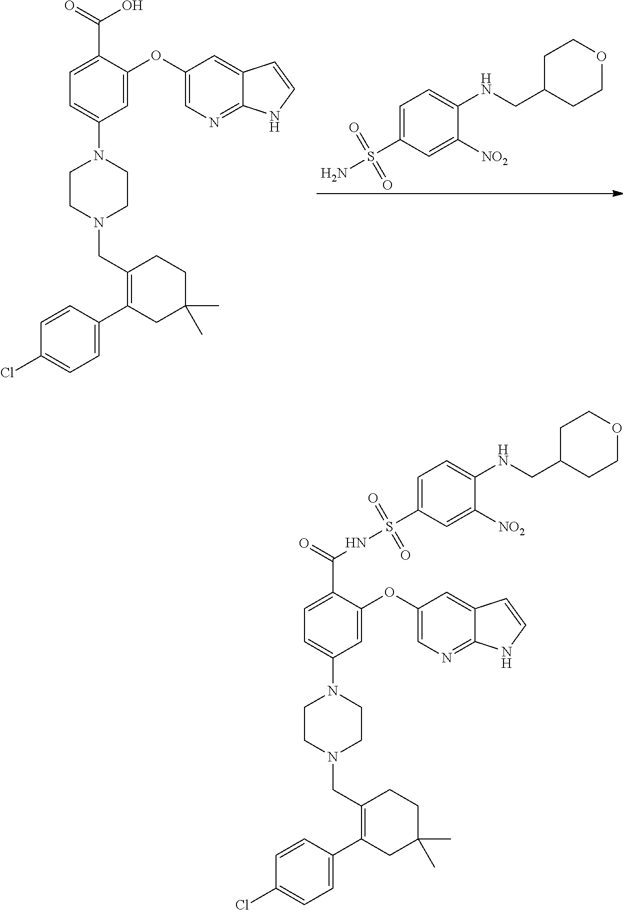 [SMILES: CC1(C)CCC(CN2CCN(c3ccc(C(=O)O)c(Oc4cnc5[nH]ccc5c4)c3)CC2)=C(c2ccc(Cl)cc2)C1 ; NS(=O)(=O)c1ccc(NCC2CCOCC2)c([N+](=O)[O-])c1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

3-nitro-4-(((tetrahydro-2H-pyran-4-yl)methyl)amino)benzenesulfonamide (4.73 g) was dissolved in dichloromethane (124.8 mL) at 34° C. 1-Ethyl-3-(3-dimethylaminopropyl)carbodiimide hydrochloride (3.66 g) and 4-Dimethylaminopyridine (3.33 g) was added to the above solution and stirred for 10 minutes at 35° C. A mixture of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (7.8 g), triethyl amine (3.8 g) in dichloromethane (70.2 mL) was stirred for 15 minutes at 35° C. and it was added drop wise to the above mixture in 15 minutes at the same temperature. The reaction mixture was stirred for 23 hours at 31° C. and then evaporated the solvent from the reaction mixture to obtain residue. This residue was dissolved in ethyl acetate (80 mL) and washed the solution with 10% acetic acid (2×80 mL), saturated aqueous sodium bicarbonate solution (2×80 mL) and then with brine solution (2×80 mL). The separated organic layer was dried over sodium sulfate and evaporated the solvent completely. The crude product was combined with acetonitrile (112 mL) and stirred for 2 hour at 34° C. and filtered the solid. The solid was dissolved in acetonitrile (60 mL) at 70° C. and stirred for 1 hour at the same temperature. The solution was cooled and filtered the solid to obtain title compound. Yield: 4.5 g; Purity by HPLC: 99.65%

### Example-44: Purification of Venetoclax

Venetoclax (13.2 g) was combined with acetonitrile and stirred the mixture for 1 hour at 70° C. Filtered the solids at 70° C. and washed with acetonitrile (66 mL) to obtain. The solid was dried under vacuum to obtain title compound. Yield: 10.26 g; Purity by HPLC: 99.53%

### Example-45: Preparation of Amorphous Form of Venetoclax

Venetoclax (40 g) was dissolved in DMSO (200 mL) at 90° C. and filtered the solution under hot condition. The hot filtrate was added to water (2000 mL) at 25° C. and stirred for about 10 minutes at the same temperature. The solid was filtered and dried under reduced pressure initially followed by drying in air tray drier at 45° C. for 9 hours and 75° C. for 7 hours to obtain the title compound. Yield: 87.5%

### Example-46: Preparation of Crystalline Form RT1 of Venetoclax

Venetoclax (11 g) was dissolved in benzyl alcohol (25 mL) at 90° C., heating was turned off after complete dissolution. n-heptane (100 mL) was added to the solution and stirred for 10 minutes. Methyl tert. butyl ether (55 mL) was added to the resultant precipitate and stirred for 10 minutes. The solid was filtered and dried under reduced pressure initially followed by drying in air tray drier at 50° C. for 2 hours to obtain the title compound. Yield: 80%.

1. A process for the preparation of an amorphous form of Venetoclax, comprising the steps of:
a) providing a solution of Venetoclax in a suitable solvent or a mixture thereof;
b) removing the solvent from the solution obtained in step a); and
c) isolating the amorphous form of Venetoclax;
d) optionally combining amorphous form of step c) with at least one pharmaceutically acceptable excipient.

2. The process according to claim 1, wherein the solvent is selected from methanol, ethanol, 2-propanol, 1-butanol, 2-butanol, 1-pentanol, 2-pentanol, 3-pentanol, dichloromethane, tetrahydrofuran, acetone, methyl ethyl ketone, methyl isobutyl ketone, methyl acetate, ethyl acetate, isopropyl acetate, water or mixtures thereof.

3. A process for the preparation of amorphous form of Venetoclax, comprising the steps of:
a) providing a solution of Venetoclax in a suitable solvent or a mixture thereof;
b) contacting the solution of step a) with an anti-solvent;
c) isolating amorphous form of Venetoclax.

4. The process according to claim 3, wherein the solvent is selected from dimethyl sulfoxide, dimethyl formamide, tetrahydrofuran or mixtures thereof.

5. The process according to claim 3, wherein the anti-solvent is selected from water, n-hexane, n-heptane, cyclohexane, di isopropyl ether, methyl tert-Butyl ether or mixtures thereof.

6. An acid addition salt of Venetoclax, wherein the acid is selected from the group comprising of trifluoro acetic acid (TFA), oxalic acid, maleic acid, Isethionic acid, ortho-phosphoric acid, citric acid, methanesulfonic acid and acetic acid.

7. A process for the preparation of salt of Venetoclax comprising the step of contacting an acid with Venetoclax, wherein acid is selected from the group comprising of trifluoro acetic acid (TFA), oxalic acid, maleic acid, isethionic acid, ortho-phosphoric acid, citric acid, methanesulfonic acid and acetic acid.

8. A process for the preparation of Venetoclax, comprising the step of converting a salt of Venetoclax into its free form, wherein the salt is selected from the group comprising of trifluoro acetic acid (TFA), oxalic acid, maleic acid, isethionic acid, ortho-phosphoric acid, citric acid, methanesulfonic acid and acetic acid.

9. A crystalline form of Venetoclax that is a:
(a) crystalline Form RT1, that exhibits an X-ray powder diffraction (XRPD) pattern with characteristic peaks at 4.39 and 8.56±0.2° 2θ;
(b) crystalline Form RT3, that exhibits an X-ray powder diffraction (XRPD) pattern with characteristic peaks at 6.30, 12.57 and 20.06±0.2° 2θ;
(c) crystalline Form RT4, that exhibits an X-ray powder diffraction (XRPD) pattern with characteristic peaks at 4.55 and 5.10±0.2° 2θ;
(d) crystalline Form RT5, that exhibits an X-ray powder diffraction (XRPD) pattern with characteristic peaks at 5.51 and 25.00±0.2° 2θ.

10. A process for the preparation of crystalline Form RT1 of Venetoclax of claim 9, comprising the step of contacting Venetoclax with benzyl alcohol.

11. A process for the preparation of crystalline Form RT2 of Venetoclax of claim 9, comprising the steps of:
a) dissolving or suspending Venetoclax in methyl isopropyl ketone or methyl-tertiary butyl ether or a mixture thereof;
b) optionally, contacting the solution of step a) with an anti-solvent;
c) isolating crystalline Form RT2 of Venetoclax.

12. The process according to claim 11, wherein the anti-solvent is selected from n-hexane, n-heptane, cyclohexane, water or mixtures thereof.

13. A process for the preparation of crystalline Form RT3 of Venetoclax of claim 9, comprising the steps of:
a) providing a solution of Venetoclax in methylene chloride or a mixture thereof;
b) optionally washing the solution of step a) with water;
c) removing the solvent of step a) to obtain crystalline Form RT3 of Venetoclax.

14. A process for the preparation of crystalline Form RT4 of Venetoclax of claim 9, comprising the steps of:
a) dissolving or suspending Venetoclax in methyl iso-butyl Ketone or a mixture thereof;
b) optionally, contacting the solution of step a) with an anti-solvent;
c) isolating crystalline Form RT4 of Venetoclax.

15. A process for the preparation of crystalline Form RT5 of Venetoclax of claim 9, comprising the steps of:
a) dissolving or suspending Venetoclax in 1,4-dioxane or mixture thereof;
b) optionally, contacting the solution of step a) with an anti-solvent;
c) isolating crystalline Form RT5 of Venetoclax.

16. The process according to claim 14, wherein the anti-solvent is selected from n-hexane, n-heptane, cyclohexane, water or mixtures thereof.

17. An amorphous solid dispersion of Venetoclax together with one or more water soluble polymers without surfactant.

18. The amorphous solid dispersion of Venetoclax according to claim 17, wherein the water soluble polymer is selected from polyvinyl pyrrolidone, povidone K-30, povidone K-60, povidone K-90, polyvinylpyrrolidone vinylacetate, co-povidone NF, polysorbate 80, polyoxyethylene-polyoxypropylene copolymers (Poloxamer 188 or pluronic F-68), polyoxyethylene (40) stearate, polyethyene glycol monomethyl ether, polyethyene glycol, hydroxypropylmethyl cellulose phthalate, hydroxypropylmethyl cellulose, hydroxypropyl cellulose SSL (HPC-SSL), hydroxypropyl cellulose SL (HPC-SL), hydroxypropyl cellulose L (HPC-L), hydroxyethyl cellulose, soluplus (polyvinyl caprolactam-polyvinyl acetate-polyethylene glycol graft copolymer (PCL-PVAc-PEG)), gelucire 44/14, cyclodextrins, gelatins, D-alpha-tocopheryl polyethylene glycol 1000 succinate, polyvinylacetal diethylaminoacetate (AEA), methylcellulose, carboxymethylethylcellulose or mixture thereof.

19. An amorphous solid dispersion of Venetoclax together with water insoluble polymer with or without surfactant.

20. The amorphous solid dispersion of Venetoclax according to claim 19, wherein the water insoluble polymer is selected from polyvinyl acetate phthalate, methacrylic acid copolymer (Eudragit or Eudragit-RLPO), hydroxypropylmethyl cellulose acetate succinate (HPMC-AS), ethyl cellulose, cellulose acetate phthalate, hypromellose phthalates, syloid or mixture thereof.

21. An amorphous solid dispersion of Venetoclax together with surfactant and without water soluble polymer.

22. The amorphous solid dispersion of Venetoclax according to claim 19, wherein the surfactant is selected from polyoxyethylene glycerides, fatty acid monoesters of sorbitan, polysorbates, α-tocopheryl polyethylene glycol succinate (TPGS) or mixtures thereof.

23. The process according to claim 15, wherein the anti-solvent is selected from n-hexane, n-heptane, cyclohexane, water or mixtures thereof.

24. The amorphous solid dispersion of Venetoclax according to claim 21, wherein the surfactant is selected from polyoxyethylene glycerides, fatty acid monoesters of sorbitan, polysorbates, α-tocopheryl polyethylene glycol succinate (TPGS) or mixtures thereof.
